(19) United States
(12) Patent Application Publication
(10) Pub. No.: US 2024/0199777 A1
(43) Pub. Date: Jun. 20, 2024

(54) AMINO-BENZIMIDAZOLE CATALYSTS FOR THE PREPARATION OF POLYOLEFINS

(71) Applicant: Dow Global Technologies LLC, Midland, MI (US)

(72) Inventors: Brad C. Bailey, Midland, MI (US); Sukrit Mukhopadhyay, Midland, MI (US)

(73) Assignee: Dow Global Technologies LLC, Midland, MI (US)

(21) Appl. No.: 18/547,963

(22) PCT Filed: Feb. 25, 2022
(86) PCT No.: PCT/US2022/017933

§ 371 Date: Aug. 25, 2023

Related U.S. Application Data
(60) Provisional application No. 63/153,980, filed on Feb. 26, 2021.

(51) Int. Cl. C08F 210/16; C07F 7/00

(52) U.S. Cl. CPC C08F 210/16; C07F 7/00

(57) ABSTRACT

Catalyst systems include a metal-ligand complex according to formula (I):

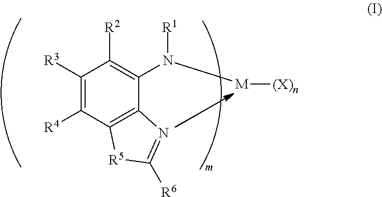

### CROSS-REFERENCE

This application claims the benefit of U.S. Provisional Patent Application Ser. No. 63/153,980 filed Feb. 26, 2021, which is hereby incorporated by reference in its entirety.

### TECHNICAL FIELD

Embodiments of the present disclosure generally relate to olefin polymerization catalyst systems and processes and, more specifically to amino-benzimdazole catalyst.

### BACKGROUND

Olefin-based polymers such as polyethylene, ethylene-based polymers, polypropylene, and propylene-based polymers are produced via various catalyst systems. Selection of such catalyst systems used in the polymerization process of the olefin-based polymers is an important factor contributing to the characteristics and properties of such olefin based polymers.

Ethylene-based polymers and propylene-based are manufactured for a wide variety of articles. The polyethylene and polypropylene polymerization process can be varied in a number of respects to produce a wide variety of resultant polyethylene resins having different physical properties that render the various resins suitable for use in different applications. The ethylene monomers and, optionally, one or more co-monomers are present in liquid diluents (such as solvents), such as an alkane or isoalkane, for example isobutene. Hydrogen may also be added to the reactor. The catalyst systems for producing ethylene-based may typically comprise a chromium-based catalyst system, a Ziegler-Natta catalyst system, and/or a molecular (either metallocene or non-metallocene (molecular)) catalyst system. The reactants in the diluent and the catalyst system are circulated at an elevated polymerization temperature around the reactor, thereby producing ethylene-based homopolymer or copolymer. Either periodically or continuously, part of the reaction mixture, including the polyethylene product dissolved in the diluent, together with unreacted ethylene and one or more optional co-monomers, is removed from the reactor. The reaction mixture, when removed from the reactor, may be processed to remove the polyethylene product from the diluent and the unreacted reactants, with the diluent and unreacted reactants typically being recycled back into the reactor. Alternatively, the reaction mixture may be sent to a second reactor, serially connected to the first reactor, where a second polyethylene fraction may be produced. Despite the research efforts in developing catalyst systems suitable for olefin polymerization, such as polyethylene or polypropylene polymerization, there is still a need to increase the efficiencies of catalyst systems that are capable of producing polymer with high molecular weights and a narrow molecular weight distribution.

### SUMMARY

1. A catalyst system comprising a metal-ligand complex according to formula (I):

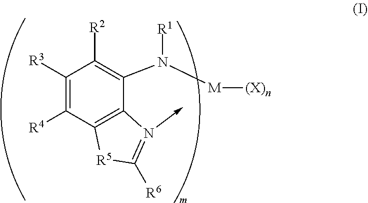

In formula (I), M is a metal chosen from titanium, zirconium, or hafnium, the metal having a formal oxidation state of +2, +3, or +4; each X is a monodentate or bidentate ligand independently chosen from unsaturated (C₂-C₅₀)hydrocarbon, unsaturated (C₂-C₅₀)heterohydrocarbon, (C₁-C₅₀)hydrocarbyl, (C₆-C₅₀)aryl, (C₆-C₅₀)heteroaryl, cyclopentadienyl, substituted cyclopentadienyl, (C₄-C₁₂)diene, halogen, and —CH₂SiR^{C}₃; wherein each R^{C} is selected from the group consisting (C₁-C₃₀)hydrocarbyl or —H. Sunscript n of (X)ₙ is 2 or 3; and subscript m is 1 or 2. The metal-ligand complex of formula (I) has 6 or fewer metal-ligand bonds.

In formula (I), each R¹ is independently selected from the group consisting of substituted (C₁-C₅₀)alkyl, unsubstituted (C₁-C₅₀)alkyl, substituted (C₆-C₅₀)aryl, and unsubstituted(C₆-C₅₀)aryl. Each R², R³, and R⁴ is independently selected from —H, (C₁-C₅₀)hydrocarbyl, (C₁-C₅₀)heterohydrocarbyl, (C₆-C₅₀)aryl, (C₄-C₅₀)heteroaryl, halogen atom, —OR^{C}, —Si(R^{C})₃, and —Ge(R^{C})₃; and each R⁵ is selected from S, —NR^{N}, or CR^{N}₂, wherein each R^{N} is (C₁-C₂₀)hydrocarbyl or —H; and each R⁶ is independently selected from —H, (C₁-C₅₀)hydrocarbyl, (C₁-C₅₀)heterohydrocarbyl, (C₆-C₅₀)aryl, (C₄-C₅₀)heteroaryl, —Si(R^{C})₃, and —Ge(R^{C})₃.

### DETAILED DESCRIPTION

Specific embodiments of catalyst systems will now be described. It should be understood that the catalyst systems of this disclosure may be embodied in different forms and should not be construed as limited to the specific embodiments set forth in this disclosure.

Common abbreviations are listed below:

R, Z, M, X and n: as defined above; Me: methyl; Et: ethyl; Ph: phenyl; Bn: benzyl; i-Pr: iso-propyl; t-Bu: tert-butyl; t-Oct: tert-octyl (2,4,4-trimethylpentan-2-yl); Tf: trifluoromethane sulfonate; CV: column volume (used in column chromatography); EtOAc: ethyl acetate; TEA: triethylaluminum; MAO: methylaluminoxane; MMAO: modified methylaluminoxane; LiCH₂TMS: (trimethylsilyl)methyllithium; TMS: trimethylsilyl; Pd(AmPhos)Cl₂: Bis(di-tert-butyl(4-dimethylaminophenyl)phosphine)dichloropalladium(II); Pd(AmPhos): Chloro(crotyl)(di-tert-butyl(4-dimethylaminophenyl)phosphine)palladium(II); Pd(dppf)Cl₂: [1,1′-Bis(diphenylphosphino)ferrocene]palladium(II) dichloride; ScCl₃: scandium(III) chloride; PhMe: toluene; THF: tetrahydrofuran; CH₂Cl₂: dichloromethane; DMF: N,N-dimethylformamide; EtOAc: ethyl acetate; Et₂O: diethyl ether; MeOH: methanol; NH₄Cl: ammonium chloride; MgSO₄: magnesium sulfate; Na₂SO₄: sodium sulfate; NaOH: sodium hydroxide; brine: saturated aqueous sodium chloride; SiO₂: silica; CDCl₃: chloroform-D; GC: gas chromatography; LC: liquid chromatography; NMR: nuclear magnetic resonance; MS: mass spectrometry; mmol: millimoles; mL: milliliters; M: molar; min or mins: minutes; h or hrs: hours; d: days; TLC; thin layered chromatography; rpm: revolution per minute; rt: room temperature.

The term “independently selected” is used herein to indicate that the R groups, such as, R¹, R², R³, R⁴, and R⁵, can be identical or different (e.g., R¹, R², R³, R⁴, and R⁵ may all be substituted alkyls or R¹ and R² may be a substituted alkyl and R³ may be an aryl, etc.) A chemical name associated with an R group is intended to convey the chemical structure that is recognized in the art as corresponding to that of the chemical name. Thus, chemical names are intended to supplement and illustrate, not preclude, the structural definitions known to those of skill in the art.

When used to describe certain carbon atom-containing chemical groups, a parenthetical expression having the form “(Cₓ-C_{y})” means that the unsubstituted form of the chemical group has from x carbon atoms to y carbon atoms, inclusive of x and y. For example, a (C₁-C₅₀)alkyl is an alkyl group having from 1 to 50 carbon atoms in its unsubstituted form. In some embodiments and general structures, certain chemical groups may be substituted by one or more substituents such as R^{S}. An R^{S} substituted chemical group defined using the “(Cₓ-C_{y})” parenthetical may contain more than y carbon atoms depending on the identity of any groups R^{S}. For example, a “(C₁-C₅₀)alkyl substituted with exactly one group R^{S}, where R^{S} is phenyl (—C₆H₅)” may contain from 7 to 56 carbon atoms. Thus, in general when a chemical group defined using the “(Cₓ-C_{y})” parenthetical is substituted by one or more carbon atom-containing substituents R^{S}, the minimum and maximum total number of carbon atoms of the chemical group is determined by adding to both x and y the combined sum of the number of carbon atoms from all of the carbon atom-containing substituents R^{S}.

The term “substitution” means that at least one hydrogen atom (—H) bonded to a carbon atom or heteroatom of a corresponding unsubstituted compound or functional group is replaced by a substituent (e.g. R^{S}). The term “persubstitution” means that every hydrogen atom (H) bonded to a carbon atom or heteroatom of a corresponding unsubstituted compound or functional group is replaced by a substituent (e.g., R^{S}). The term “polysubstitution” means that at least two, but fewer than all, hydrogen atoms bonded to carbon atoms or heteroatoms of a corresponding unsubstituted compound or functional group are replaced by a substituent. The term “—H” means a hydrogen or hydrogen radical that is covalently bonded to another atom. “Hydrogen” and “—H” are interchangeable, and unless clearly specified have identical meanings.

The term “(C₁-C₅₀)hydrocarbyl” means a hydrocarbon radical of from 1 to 50 carbon atoms and the term “(C₁-C₅₀)hydrocarbylene” means a hydrocarbon diradical of from 1 to 50 carbon atoms, in which each hydrocarbon radical and each hydrocarbon diradical is aromatic or non-aromatic, saturated or unsaturated, straight chain or branched chain, cyclic (having three carbons or more, and including mono- and poly-cyclic, fused and non-fused polycyclic, and bicyclic) or acyclic, and substituted by one or more R^{S} or unsubstituted.

In this disclosure, a (C₁-C₅₀)hydrocarbyl may be an unsubstituted or substituted (C₁-C₅₀)alkyl, (C₃-C₅₀)cycloalkyl, (C₃-C₂₀)cycloalkyl-(C₁-C₂₀)alkylene, (C₆-C₄₀)aryl, or (C₆-C₂₀)aryl-(C₁-C₂₀)alkylene (such as benzyl (—CH₂—C₆H₅)).

The terms “(C₁-C₅₀)alkyl” and “(C₁-C₁₈)alkyl” mean a saturated straight or branched hydrocarbon radical of from 1 to 50 carbon atoms and a saturated straight or branched hydrocarbon radical of from 1 to 18 carbon atoms, respectively, that is unsubstituted or substituted by one or more R^{S}. Examples of unsubstituted (C₁-C₅₀)alkyl are unsubstituted (C₁-C₂₀)alkyl; unsubstituted (C₁-C₁₀)alkyl; unsubstituted (C₁-C₅)alkyl; methyl; ethyl; 1-propyl; 2-propyl; 1-butyl; 2-butyl; 2-methylpropyl; 1,1-dimethylethyl; 1-pentyl; 1-hexyl; 1-heptyl; 1-nonyl; and 1-decyl. Examples of substituted (C₁-C₄₀)alkyl are substituted (C₁-C₂₀)alkyl, substituted (C₁-C₁₀)alkyl, trifluoromethyl, and [C₄₅]alkyl. The term “[C₄₅]alkyl” means there is a maximum of 45 carbon atoms in the radical, including substituents, and is, for example, a (C₂₇-C₄₀)alkyl substituted by one R^{S}, which is a (C₁-C₅)alkyl, respectively. Each (C₁-C₅)alkyl may be methyl, trifluoromethyl, ethyl, 1-propyl, 1-methylethyl, or 1,1-dimethylethyl.

The term “(C₆-C₅₀)aryl” means an unsubstituted or substituted (by one or more R^{S}) monocyclic, bicyclic, or tricyclic aromatic hydrocarbon radical of from 6 to 40 carbon atoms, of which at least from 6 to 14 of the carbon atoms are aromatic ring carbon atoms. A monocyclic aromatic hydrocarbon radical includes one aromatic ring; a bicyclic aromatic hydrocarbon radical has two rings; and a tricyclic aromatic hydrocarbon radical has three rings. When the bicyclic or tricyclic aromatic hydrocarbon radical is present, at least one of the rings of the radical is aromatic. The other ring or rings of the aromatic radical may be independently fused or non-fused and aromatic or non-aromatic. Examples of unsubstituted (C₆-C₅₀)aryl include: unsubstituted (C₆-C₂₀)aryl, unsubstituted (C₆-C₁₈)aryl; 2-(C₁-C₅)alkyl-phenyl; phenyl; fluorenyl; tetrahydrofluorenyl; indacenyl; hexahydroindacenyl; indenyl; dihydroindenyl; naphthyl; tetrahydronaphthyl; and phenanthrene. Examples of substituted (C₆-C₄₀)aryl include: substituted (C₁-C₂₀)aryl; substituted (C₆-C₁₅)aryl; 2,4-bis([C₂₀]alkyl)-phenyl; polyfluorophenyl; pentafluorophenyl; and fluoren-9-one-1-yl.

The term “(C₃-C₅₀)cycloalkyl” means a saturated cyclic hydrocarbon radical of from 3 to 50 carbon atoms that is unsubstituted or substituted by one or more R^{S}. Other cycloalkyl groups (e.g., (Cₓ-C_{y})cycloalkyl) are defined in an analogous manner as having from x to y carbon atoms and being either unsubstituted or substituted with one or more R^{S}. Examples of unsubstituted (C₃-C₄₀)cycloalkyl are unsubstituted (C₃-C₂₀)cycloalkyl, unsubstituted (C₃-C₁₀)cycloalkyl, cyclopropyl, cyclobutyl, cyclopentyl, cyclohexyl, cycloheptyl, cyclooctyl, cyclononyl, and cyclodecyl. Examples of substituted (C₃-C₄₀)cycloalkyl are substituted (C₃-C₂₀)cycloalkyl, substituted (C₃-C₁₀)cycloalkyl, cyclopentanon-2-yl, and 1-fluorocyclohexyl.

Examples of (C₁-C₅₀)hydrocarbylene include unsubstituted or substituted (C₆-C₅₀)arylene, (C₃-C₅₀)cycloalkylene, and (C₁-C₅₀)alkylene (e.g., (C₁-C₂₀)alkylene). The diradicals may be on the same carbon atom (e.g., —CH₂—) or on adjacent carbon atoms (i.e., 1,2-diradicals), or are spaced apart by one, two, or more than two intervening carbon atoms (e.g., 1,3-diradicals, 1,4-diradicals, etc.). Some diradicals include 1,2-, 1,3-, 1,4-, or an α,ω-diradical, and others a 1,2-diradical. The α,ω-diradical is a diradical that has maximum carbon backbone spacing between the radical carbons. Some examples of (C₂-C₂₀)alkylene α,ω-diradicals include ethan-1,2-diyl (i.e. —CH₂CH₂—), propan-1,3-diyl (i.e. —CH₂CH₂CH₂—), 2-methylpropan-1,3-diyl (i.e. —CH₂CH(CH₃)CH₂—). Some examples of (C₆-C₅₀)arylene α,ω-diradicals include phenyl-1,4-diyl, napthalen-2,6-diyl, or napthalen-3,7-diyl.

The term “(C₁-C₅₀)alkylene” means a saturated straight chain or branched chain diradical (i.e., the radicals are not on ring atoms) of from 1 to 50 carbon atoms that is unsubstituted or substituted by one or more R^{S}. Examples of unsubstituted (C₁-C₅₀)alkylene are unsubstituted (C₁-C₂₀)alkylene, including unsubstituted —CH₂CH₂—, —(CH₂)₃—, —(CH₂)₄—, —(CH₂)₅—, —(CH₂)₆—, —(CH₂)₇—, —(CH₂)₈—, —CH₂C*HCH₃, and —(CH₂)₄C*(H)(CH₃), in which “C*” denotes a carbon atom from which a hydrogen atom is removed to form a secondary or tertiary alkyl radical. Examples of substituted (C₁-C₅₀)alkylene are substituted (C₁-C₂₀)alkylene, —CF₂—, —C(O)—, and —(CH₂)₁₄C(CH₃)₂(CH₂)₅— (i.e., a 6,6-dimethyl substituted normal-1,20-eicosylene). Since as mentioned previously two R^{S} may be taken together to form a (C₁-C₁₈)alkylene, examples of substituted (C₁-C₅₀)alkylene also include 1,2-bis(methylene)cyclopentane, 1,2-bis(methylene)cyclohexane, 2,3-bis(methylene)-7,7-dimethyl-bicyclo[2.2.1]heptane, and 2,3-bis (methylene)bicyclo [2.2.2] octane.

The term “(C₃-C₅₀)cycloalkylene” means a cyclic diradical (i.e., the radicals are on ring atoms) of from 3 to 50 carbon atoms that either is unsubstituted or is substituted by one or more R^{S}.

The term “heteroatom,” refers to an atom other than hydrogen or carbon. Examples of groups containing one or more than one heteroatom include O, S. S(O), S(O)₂. Si(R^{C})₂, P(R^{P}), N(R^{N}), —N═C(R^{C})₂, —Ge(R^{C})₂—, —Si(R^{C})—, boron (B), aluminum (Al), gallium (Ga), or indium (In), where each R^{C} and each R^{P} is unsubstituted (C₁-C₁₈)hydrocarbyl or —H, and where each R^{N} is unsubstituted (C₁-C₁₈)hydrocarbyl. The term “heterohydrocarbon” refers to a molecule or molecular framework in which one or more carbon atoms of a hydrocarbon are replaced with a heteroatom. The term “(C₁-C₅₀)heterohydrocarbyl” means a heterohydrocarbon radical of from 1 to 50 carbon atoms, and the term “(C₁-C₅₀)heterohydrocarbylene” means a heterohydrocarbon diradical of from 1 to 50 carbon atoms. The heterohydrocarbon of the (C₁-C₅₀)heterohydrocarbyl or the (C₁-C₅₀)heterohydrocarbylene has one or more heteroatoms. The radical of the heterohydrocarbyl may be on a carbon atom or a heteroatom. The two radicals of the heterohydrocarbylene may be on a single carbon atom or on a single heteroatom. Additionally, one of the two radicals of the diradical may be on a carbon atom and the other radical may be on a different carbon atom; one of the two radicals may be on a carbon atom and the other on a heteroatom; or one of the two radicals may be on a heteroatom and the other radical on a different heteroatom. Each (C₁-C₅₀)heterohydrocarbyl and (C₁-C₅₀)heterohydrocarbylene may be unsubstituted or substituted (by one or more R^{S}), aromatic or non-aromatic, saturated or unsaturated, straight chain or branched chain, cyclic (including mono- and poly-cyclic, fused and non-fused polycyclic), or acyclic.

The (C₁-C₅₀)heterohydrocarbyl may be unsubstituted or substituted. Non-limiting examples of the (C₁-C₅₀)heterohydrocarbyl include (C₁-C₅₀)heteroalkyl, (C₁-C₅₀)hydrocarbyl-O—, (C₁-C₅₀)hydrocarbyl-S—, (C₁-C₅₀)hydrocarbyl-S(O)—, (C₁-C₅₀)hydrocarbyl-S(O)₂—, (C₁-C₅₀)hydrocarbyl-Si(R^{C})₂—, (C₁-C₅₀)hydrocarbyl-N(R^{N})—, (C₁-C₅₀)hydrocarbyl-P(R^{P})—, (C₂-C₅₀)heterocycloalkyl, (C₂-C₁₉)heterocycloalkyl-(C₁-C₂₀)alkylene, (C₃-C₂₀)cycloalkyl-(C₁-C₁₉)heteroalkylene, (C₂-C₁₉)heterocycloalkyl-(C₁-C₂₀)heteroalkylene, (C₁-C₅₀)heteroaryl, (C₁-C₁₉)heteroaryl-(C₁-C₂₀)alkylene, (C₆-C₂₀)aryl-(C₁-C₁₉)heteroalkylene, or (C₁-C₁₉)heteroaryl-(C₁-C₂₀)heteroalkylene.

The term “(C₁-C₅₀)heteroaryl” means an unsubstituted or substituted (by one or more R^{S}) mono-, bi-, or tricyclic heteroaromatic hydrocarbon radical of from 1 to 50 total carbon atoms and from 1 to 10 heteroatoms. A monocyclic heteroaromatic hydrocarbon radical includes one heteroaromatic ring; a bicyclic heteroaromatic hydrocarbon radical has two rings; and a tricyclic heteroaromatic hydrocarbon radical has three rings. When the bicyclic or tricyclic heteroaromatic hydrocarbon radical is present, at least one of the rings in the radical is heteroaromatic. The other ring or rings of the heteroaromatic radical may be independently fused or non-fused and aromatic or non-aromatic. Other heteroaryl groups (e.g., (Cₓ-C_{y})heteroaryl generally, such as (C₁-C₁₂)heteroaryl) are defined in an analogous manner as having from x to y carbon atoms (such as 1 to 12 carbon atoms) and being unsubstituted or substituted by one or more than one R^{S}. The monocyclic heteroaromatic hydrocarbon radical is a 5-membered ring or a 6-membered ring. The 5-membered ring monocyclic heteroaromatic hydrocarbon radical has 5 minus h carbon atoms, where h is the number of heteroatoms and may be 1, 2, 3, or 4; and each heteroatom may be O, S, N. or P. Examples of 5-membered ring heteroaromatic hydrocarbon radicals include pyrrol-1-yl; pyrrol-2-yl; furan-3-yl; thiophen-2-yl; pyrazol-1-yl; isoxazol-2-yl; isothiazol-5-yl; imidazol-2-yl; oxazol-4-yl; thiazol-2-yl; 1,2,4-triazol-1-yl; 1,3,4-oxadiazol-2-yl; 1,3,4-thiadiazol-2-yl; tetrazol-1-yl; tetrazol-2-yl; and tetrazol-5-yl. The 6-membered ring monocyclic heteroaromatic hydrocarbon radical has 6 minus h carbon atoms, where h is the number of heteroatoms and may be 1 or 2 and the heteroatoms may be N or P. Examples of 6-membered ring heteroaromatic hydrocarbon radicals include pyridine-2-yl; pyrimidin-2-yl; and pyrazin-2-yl. The bicyclic heteroaromatic hydrocarbon radical can be a fused 5,6- or 6,6-ring system. Examples of the fused 5,6-ring system bicyclic heteroaromatic hydrocarbon radical are indol-1-yl; and benzimidazole-1-yl. Examples of the fused 6,6-ring system bicyclic heteroaromatic hydrocarbon radical are quinolin-2-yl; and isoquinolin-1-yl. The tricyclic heteroaromatic hydrocarbon radical can be a fused 5,6,5-; 5,6,6-; 6,5,6-; or 6,6,6-ring system. An example of the fused 5,6,5-ring system is 1,7-dihydropyrrolo[3,2-f]indol-1-yl. An example of the fused 5,6,6-ring system is 1H-benzo[f] indol-1-yl. An example of the fused 6,5,6-ring system is 9H-carbazol-9-yl. An example of the fused 6,5,6-ring system is 9H-carbazol-9-yl. An example of the fused 6,6,6-ring system is acrydin-9-yl.

The term “(C₁-C₅₀)heteroalkyl” means a saturated straight or branched chain radical containing one to fifty carbon atoms and one or more heteroatom. The term “(C₁-C₅₀)heteroalkylene” means a saturated straight or branched chain diradical containing from 1 to 50 carbon atoms and one or more than one heteroatoms. The heteroatoms of the heteroalkyls or the heteroalkylenes may include Si(R^{C})₃, Ge(R^{C})₃, Si(R^{C})₂, Ge(R^{C})₂, P(R^{P})₂, P(R^{P}), N(R^{N})₂, N(R^{N}), N, O, OR^{C}, S, SR^{C}, S(O), and S(O)₂, wherein each of the heteroalkyl and heteroalkylene groups are unsubstituted or are substituted by one or more R^{S}.

Examples of unsubstituted (C₂-C₄₀)heterocycloalkyl include unsubstituted (C₂-C₂₀)heterocycloalkyl, unsubstituted (C₂-C₁₀)heterocycloalkyl, aziridin-1-yl, oxetan-2-yl, tetrahydrofuran-3-yl, pyrrolidin-1-yl, tetrahydrothiophen-S,S-dioxide-2-yl, morpholin-4-yl, 1,4-dioxan-2-yl, hexahydroazepin-4-yl, 3-oxa-cyclooctyl, 5-thio-cyclononyl, and 2-aza-cyclodecyl.

The term “halogen atom” or “halogen” means the radical of a fluorine atom (F), chlorine atom (Cl), bromine atom (Br), or iodine atom (I). The term “halide” means anionic form of the halogen atom: fluoride (F⁻), chloride (Cl⁻), bromide (Br⁻), or iodide (I⁻).

The term “saturated” means lacking carbon-carbon double bonds, carbon-carbon triple bonds, and (in heteroatom-containing groups) carbon-nitrogen, carbon-phosphorous, and carbon-silicon double bonds. Where a saturated chemical group is substituted by one or more substituents R^{S}, one or more double and/or triple bonds optionally may be present in substituents R^{S}. The term “unsaturated” means containing one or more carbon-carbon double bonds or carbon-carbon triple bonds, or (in heteroatom-containing groups) one or more carbon-nitrogen double bonds, carbon-phosphorous double bonds, or carbon-silicon double bonds, not including double bonds that may be present in substituents R^{S}, if any, or in aromatic rings or heteroaromatic rings, if any.

Embodiments of this disclosure include one or more catalyst systems. The catalyst systems include one or more metal-ligand complexes according to formula (I):

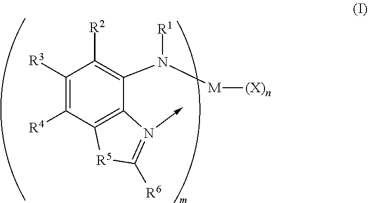

In formula (I), M is a metal chosen from titanium, zirconium, or hafnium, the metal having a formal oxidation state of +2, +3, or +4; each X is a monodentate or bidentate ligand independently chosen from unsaturated (C₂-C₅₀)hydrocarbon, unsaturated (C₂-C₅₀)heterohydrocarbon, (C₁-C₅₀)hydrocarbyl, (C₆-C₅₀)aryl, (C₆-C₅₀)heteroaryl, cyclopentadienyl, substituted cyclopentadienyl, (C₄-C₁₂)diene, halogen, and —CH₂SiR^{C}₃; wherein each R^{C} is selected from the group consisting (C₁-C₃₀)hydrocarbyl or —H. In formula (I), subscript n of (X) is 2 or 3 and subscript m is 1 or 2. The metal-ligand complex of formula (I) has 6 or fewer metal-ligand bonds.

In formula (I), each R¹ is independently selected from the group consisting of (C₁-C₅₀)alkyl or (C₆-C₅₀)aryl; each R², R³, and R⁴ is independently selected from —H, (C₁-C₅₀)hydrocarbyl, (C₁-C₅₀)heterohydrocarbyl, (C₆-C₅₀)aryl, (C₄-C₅₀)heteroaryl, —OR^{C}, —Si(R^{C})₃, and —Ge(R^{C})₃; each R⁵ is selected from S, —NR^{N}, or CR^{N}₂, wherein each R^{N} is (C₁-C₂₀)hydrocarbyl or —H; and each R⁶ is independently selected from —H, (C₁-C₅₀)hydrocarbyl, (C₁-C₅₀)heterohydrocarbyl, (C₆-C₅₀)aryl, (C₄-C₅₀)heteroaryl, —Si(R^{C})₃, and —Ge(R^{C})₃.

In one or more embodiments, the metal-ligand complex of formula (I) M is zirconium or hafnium; each X is independently chosen from unsubstituted (C₁-C₁₀)alkyl, substituted (C₁-C₁₀)alkyl, (C₆-C₂₀)aryl or a halogen; and each R¹ is independently chosen from (C₆-C₅₀)aryl or (C₁-C₅₀)alkyl.

In some embodiments, each R³, R⁴, and R⁵ is —H.

In various embodiments, each R¹ is unsubstituted phenyl, substituted phenyl, unsubstituted anthracenyl, substituted anthracenyl, unsubstituted napthyl, or substituted naphtyl. In one or more embodiments, each R¹ is a substituted phenyl; the substituted phenyl is chosen from 2-methylphenyl, 2-(iso-propyl)phenyl, 2,4,6-trimethylphenyl, 2,6-di(iso-propyl)phenyl, 2,4,6-tri(iso-propyl)phenyl, 3,5-di-tert-butylphenyl, 3,5-diphenylphenyl, 2,3,5,6-tetra-fluorophenyl.

In various embodiments, R⁵ is NR^{N}, where R^{N} is (C₁-C₂₀)alkyl or (C₆-C₂₀)aryl; in some embodiments, R^{N} is a linear (C₁-C₁₂)alkyl.

In embodiments, the metal-ligand complex may include two bidentate ligand, in which m is 2 and the metal-ligand complex has a structure according to formula (II):

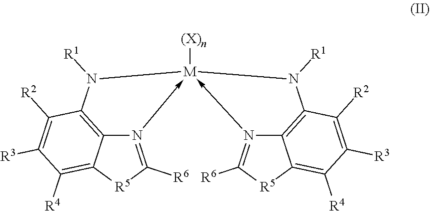

In formula (II), each R¹, R², R³, R⁴, R⁵, R⁶, and X are as defined in formula (I); and n is 1 or 2.

In the metal-ligand complex according to formula (I) or formula (II), each X bonds with M through a covalent bond, a dative bond, or an ionic bond. In some embodiments, each X is identical. The metal-ligand complex has 6 or fewer metal-ligand bonds and can be overall charge-neutral or may have a positive-charge associated with the metal center. In some embodiments, the catalyst system includes a metal-ligand complex according to formula (I), in which M is zirconium or hafnium; each X is independently chosen from (C₁-C₂₀)alkyl, (C₁-C₂₀)heteroalkyl, (C₆-C₂₀)aryl, (C₄-C₂₀)heteroaryl, (C₄-C₁₂)diene, or a halogen. In one or more embodiments, each X is independently benzyl, phenyl, or chloro.

In some embodiments, the monodentate ligand may be a monoanionic ligand. Monoanionic ligands have a net formal oxidation state of −1. Each monoanionic ligand may independently be hydride, (C₁-C₄₀)hydrocarbyl carbanion, (C₁-C₄₀)heterohydrocarbyl carbanion, halide, nitrate, carbonate, phosphate, sulfate, HC(O)O⁻, HC(O)N(H)⁻, (C₁-C₄₀)hydrocarbylC(O)O⁻, (C₁-C₄₀)hydrocarbylC(O)N((C₁-C₂₀)hydrocarbyl)⁻, (C₁-C₄₀)hydrocarbylC(O)N(H)⁻, R^{K}R^{L}B⁻, R^{K}R^{L}N⁻, R^{K}O⁻, R^{K}S⁻, R^{K}R^{L}P⁻, or R^{M}R^{K}R^{L}Si⁻, where each R^{K}, R^{L}, and R^{M} independently is hydrogen, (C₁-C₄₀)hydrocarbyl, or (C₁-C₄₀)heterohydrocarbyl, or R^{K} and R^{L} are taken together to form a (C₂-C₄₀)hydrocarbylene or (C₁-C₂₀)heterohydrocarbylene and R^{M} is as defined above.

In other embodiments, at least one monodentate ligand X, independently from any other ligands X, may be a neutral ligand. In specific embodiments, the neutral ligand is a neutral Lewis base group such as R^{Q}NR^{K}R^{L}, R^{K}OR^{L}. R^{K}SR^{L}, or R^{Q}PR^{K}R^{L}, where each R^{Q} independently is hydrogen, [(C₁-C₁₀)hydrocarbyl]₃Si(C₁-C₁₀)hydrocarbyl, (C₁-C₄₀)hydrocarbyl, [(C₁-C₁₀)hydrocarbyl]₃Si, or (C₁-C₄₀)heterohydrocarbyl and each R^{K} and R^{L} independently is as previously defined.

Additionally, each X can be a monodentate ligand that, independently from any other ligands X, is a halogen, unsubstituted (C₁-C₂₀)hydrocarbyl, unsubstituted (C₁-C₂₀)hydrocarbylC(O)O—, or R^{K}R^{L}N—, wherein each of R^{K} and R^{L} independently is an unsubstituted(C₁-C₂₀)hydrocarbyl. In some embodiments, each monodentate ligand X is a chlorine atom, (C₁-C₁₀)hydrocarbyl (e.g., (C₁-C₆)alkyl or benzyl), unsubstituted (C₁-C₁₀)hydrocarbylC(O)O—, or R^{K}R^{L}N—, wherein each of R^{K} and R^{L} independently is an unsubstituted (C₁-C₁₀)hydrocarbyl. In one or more embodiments of formula (I), (II), and (III), X is benzyl, chloro, —CH₂SiMe₃, or phenyl.

In further embodiments, each X is selected from methyl; ethyl; 1-propyl; 2-propyl; 1-butyl; 2,2-dimethylpropyl; trimethylsilylmethyl; phenyl; benzyl; or chloro. In some embodiments, each X is the same. In other embodiments, at least two X are different from each other. In the embodiments in which at least two X are different from at least one X. X is a different one of methyl; ethyl; 1-propyl; 2-propyl; 1-butyl; 2,2-dimethylpropyl; trimethylsilylmethyl; phenyl; benzyl; and chloro. In further embodiments, the bidentate ligand is 2,2-dimethyl-2-silapropane-1,3-diyl or 1,3-butadiene.

In some embodiments, any or all of the chemical groups (e.g., X and R¹-R⁴) of the metal-ligand complex of formula (I) may be unsubstituted. In other embodiments, none, any, or all of the chemical groups X and R¹-R⁴ of the metal-ligand complex of formula (I) may be substituted with one or more than one R^{S}. When two or more than two R^{S} are bonded to a same chemical group of the metal-ligand complex of formula (I), the individual R^{S} of the chemical group may be bonded to the same carbon atom or heteroatom or to different carbon atoms or heteroatoms. In some embodiments, none, any, or all of the chemical groups X and R¹-R⁴ may be persubstituted with R^{S}. In the chemical groups that are persubstituted with R^{S}, the individual R^{S} may all be the same or may be independently chosen.

In illustrative embodiments, the catalyst systems may include a metal-ligand complex according to formula (I) having the structure of any of the Metal-Ligand 1-13 listed below:

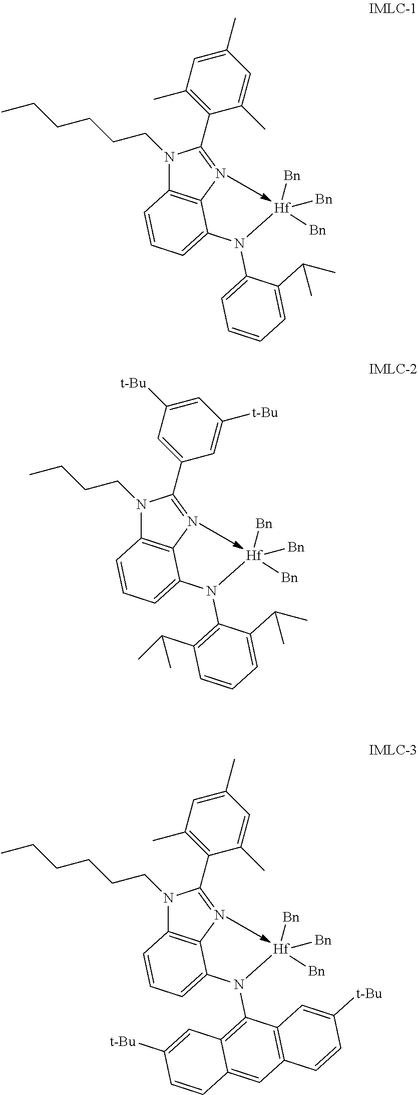

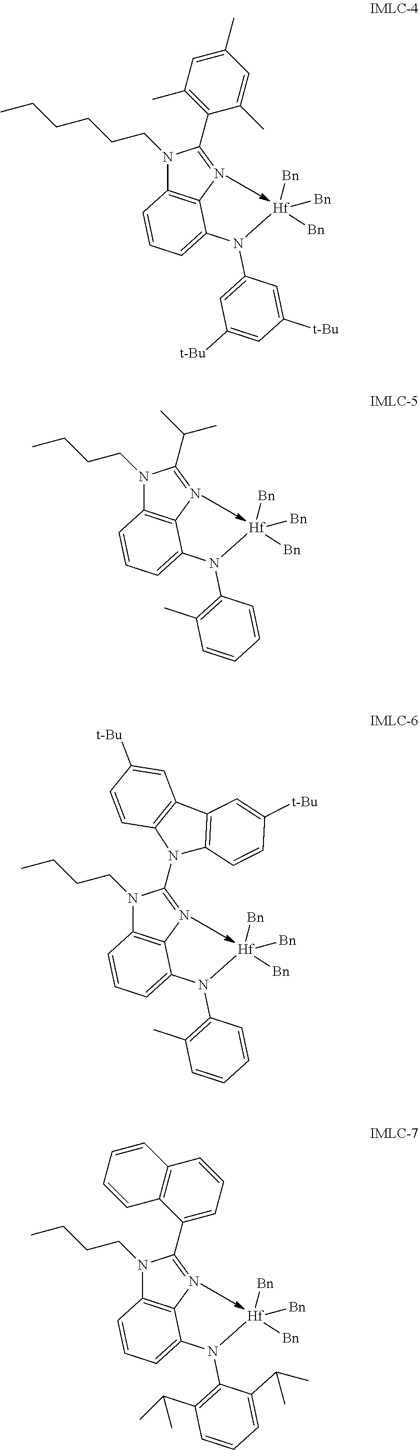

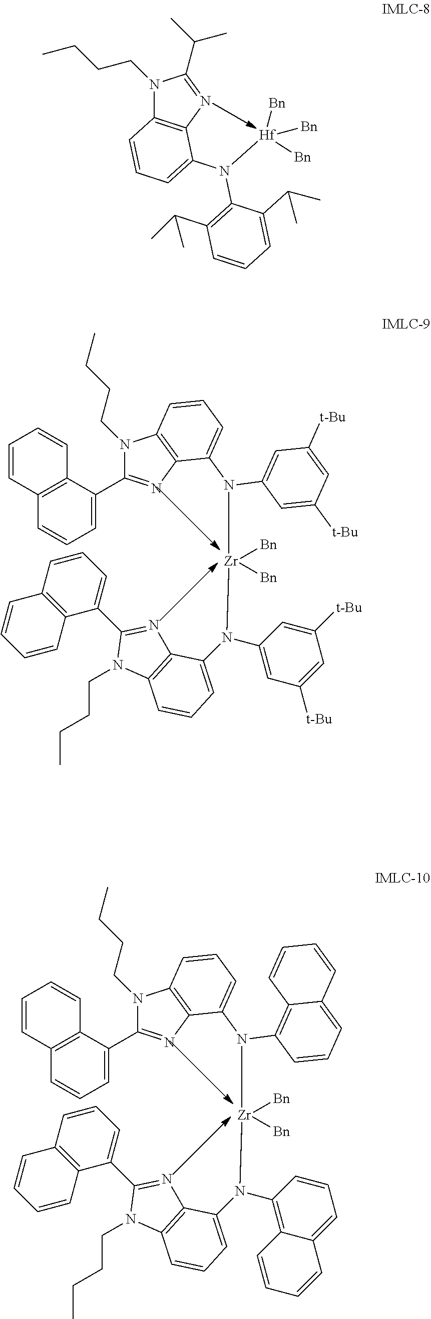

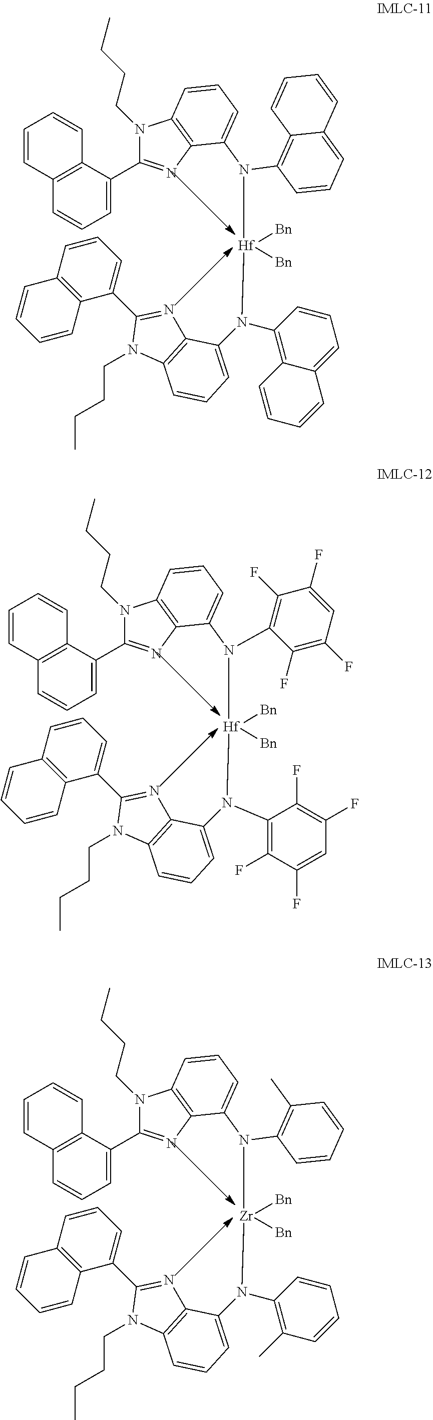

In illustrative embodiments, the catalyst systems may include a metal-ligand complex according to formula (I) having the structure of any of the Metal-ligand complex 1 to 13 or metal-ligand complexes form in situ which are synthesized from the corresponding ligands below:

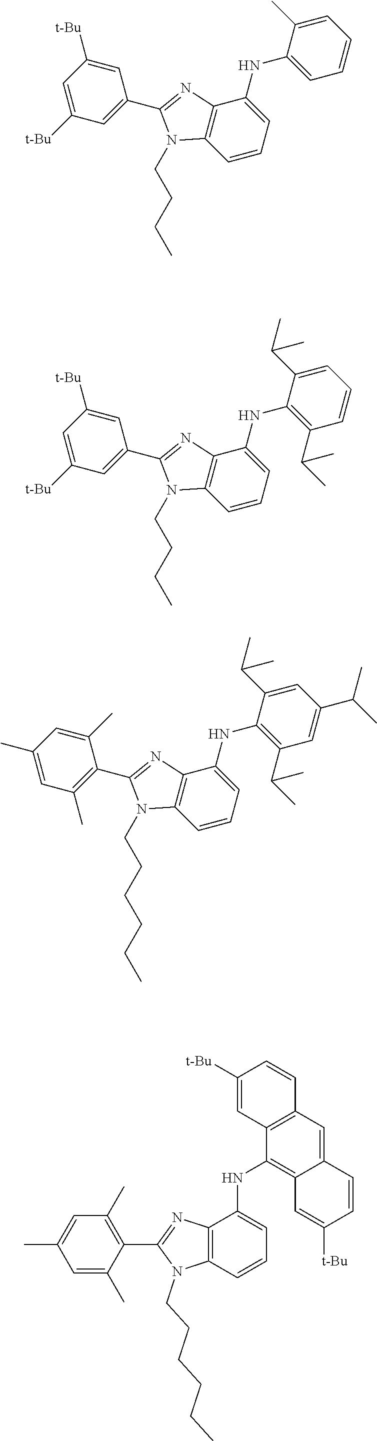

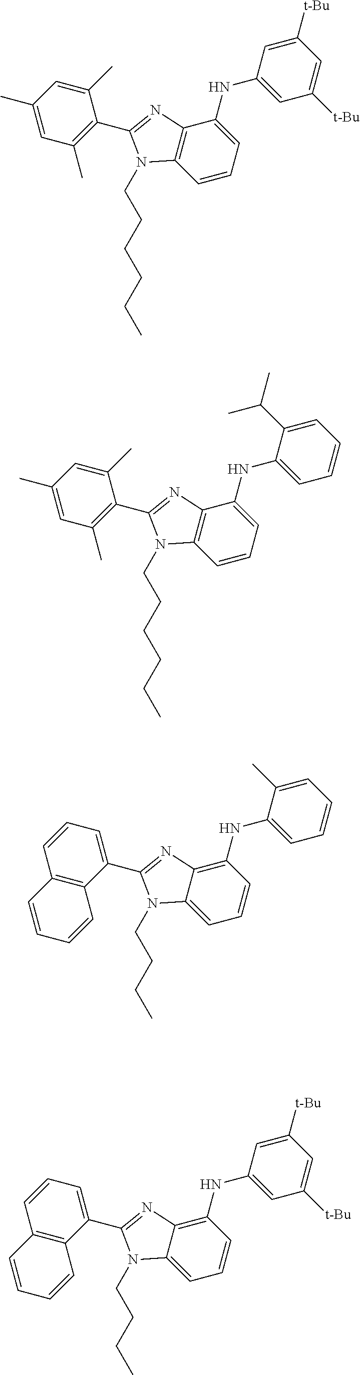 [SMILES: CCCCCCn1c(-c2c(C)cc(C)cc2C)nc2c(Nc3cc(C(C)(C)C)cc(C(C)(C)C)c3)cccc21 ; CCCCCCn1c(-c2c(C)cc(C)cc2C)nc2c(Nc3ccccc3C(C)C)cccc21 ; CCCCn1c(-c2cccc3ccccc23)nc2c(Nc3ccccc3C)cccc21 ; CCCCn1c(-c2cccc3ccccc23)nc2c(Nc3cc(C(C)(C)C)cc(C(C)(C)C)c3)cccc21]

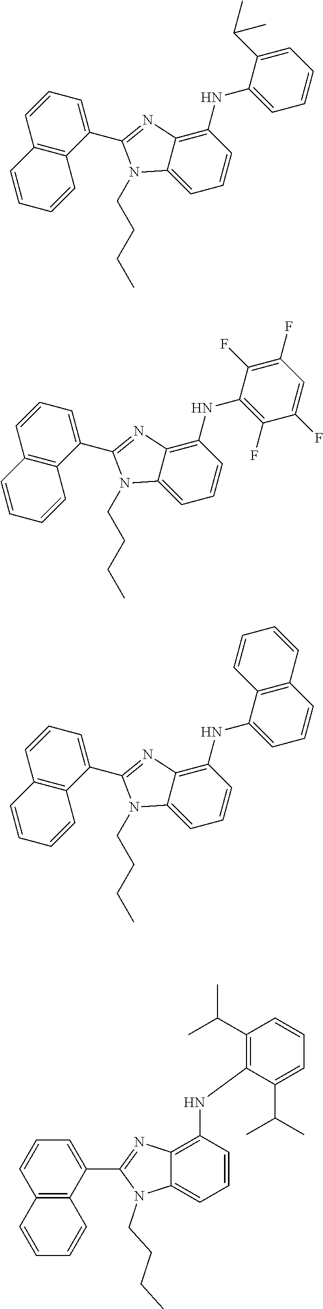 [SMILES: CCCCn1c(-c2cccc3ccccc23)nc2c(Nc3ccccc3C(C)C)cccc21 ; CCCCn1c(-c2cccc3ccccc23)nc2c(Nc3c(F)c(F)cc(F)c3F)cccc21 ; CCCCn1c(-c2cccc3ccccc23)nc2c(Nc3cccc4ccccc34)cccc21 ; CCCCn1c(-c2cccc3ccccc23)nc2c(Nc3c(C(C)C)cccc3C(C)C)cccc21]

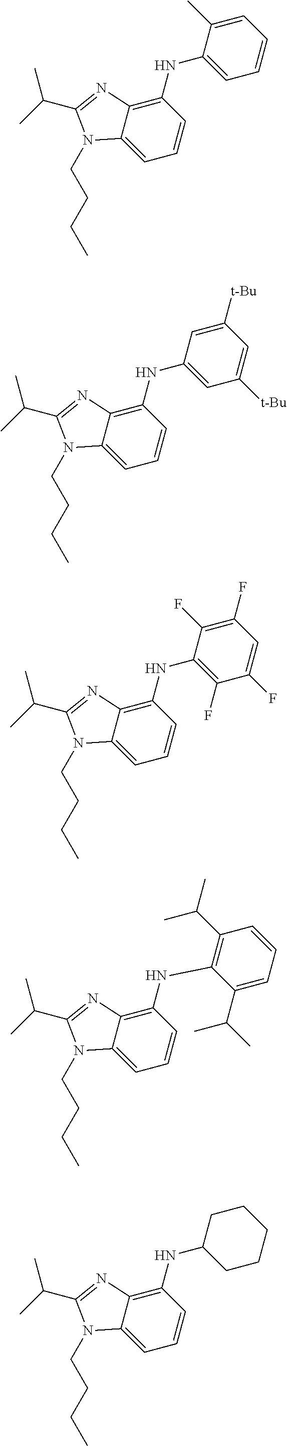 [SMILES: CCCCn1c(C(C)C)nc2c(Nc3ccccc3C)cccc21 ; CCCCn1c(C(C)C)nc2c(Nc3cc(C(C)(C)C)cc(C(C)(C)C)c3)cccc21 ; CCCCn1c(C(C)C)nc2c(Nc3c(F)c(F)cc(F)c3F)cccc21 ; CCCCn1c(C(C)C)nc2c(Nc3c(C(C)C)cccc3C(C)C)cccc21 ; CCCCn1c(C(C)C)nc2c(NC3CCCCC3)cccc21]

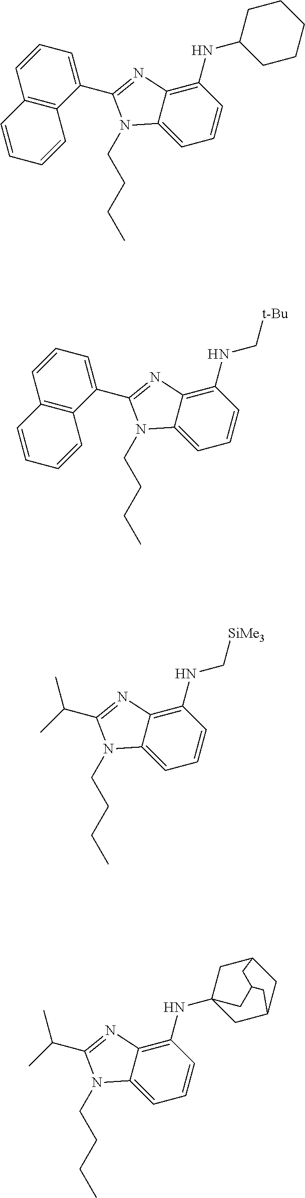 [SMILES: CCCCn1c(-c2cccc3ccccc23)nc2c(NC3CCCCC3)cccc21 ; CCCCn1c(-c2cccc3ccccc23)nc2c(NCC(C)(C)C)cccc21 ; CCCCn1c(C(C)C)nc2c(NC[Si](C)(C)C)cccc21 ; CCCCn1c(C(C)C)nc2c(NC34CC5CC(CC(C5)C3)C4)cccc21]

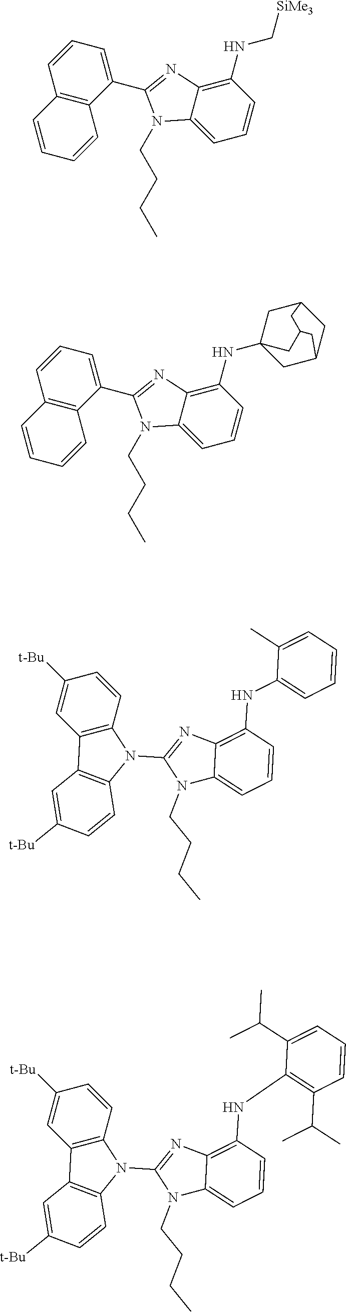 [SMILES: CCCCn1c(-c2cccc3ccccc23)nc2c(NC[Si](C)(C)C)cccc21 ; CCCCn1c(-c2cccc3ccccc23)nc2c(NC34CC5CC(CC(C5)C3)C4)cccc21 ; CCCCn1c(-n2c3ccc(C(C)(C)C)cc3c3cc(C(C)(C)C)ccc32)nc2c(Nc3ccccc3C)cccc21 ; CCCCn1c(-n2c3ccc(C(C)(C)C)cc3c3cc(C(C)(C)C)ccc32)nc2c(Nc3c(C(C)C)cccc3C(C)C)cccc21]

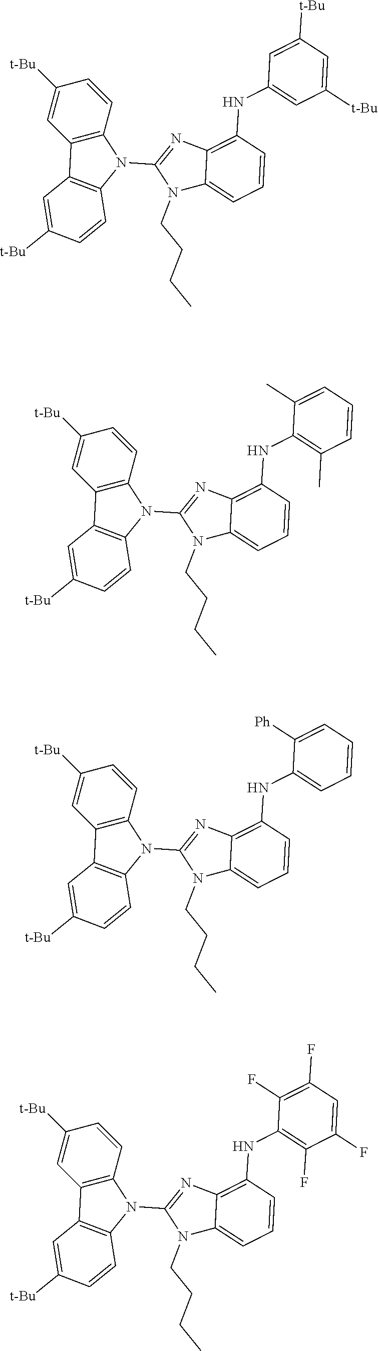 [SMILES: CCCCn1c(-n2c3ccc(C(C)(C)C)cc3c3cc(C(C)(C)C)ccc32)nc2c(Nc3cc(C(C)(C)C)cc(C(C)(C)C)c3)cccc21 ; CCCCn1c(-n2c3ccc(C(C)(C)C)cc3c3cc(C(C)(C)C)ccc32)nc2c(Nc3c(C)cccc3C)cccc21 ; CCCCn1c(-n2c3ccc(C(C)(C)C)cc3c3cc(C(C)(C)C)ccc32)nc2c(Nc3ccccc3-c3ccccc3)cccc21 ; CCCCn1c(-n2c3ccc(C(C)(C)C)cc3c3cc(C(C)(C)C)ccc32)nc2c(Nc3c(F)c(F)cc(F)c3F)cccc21]

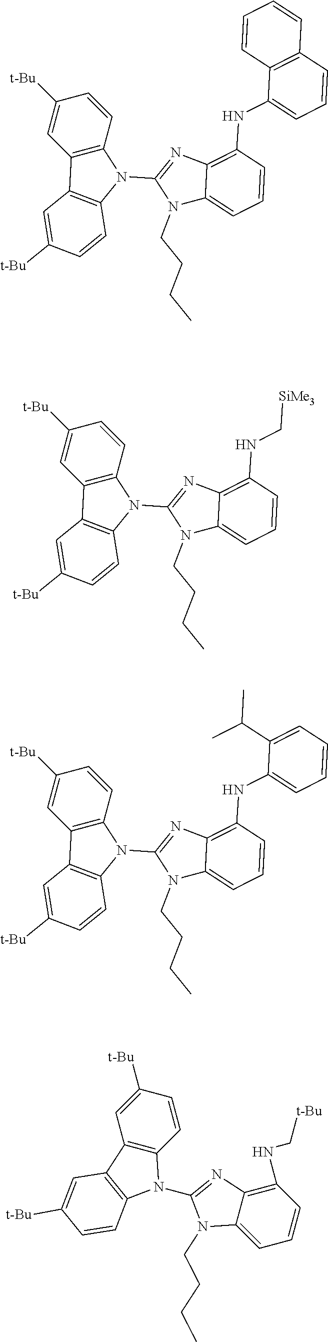 [SMILES: CCCCn1c(-n2c3ccc(C(C)(C)C)cc3c3cc(C(C)(C)C)ccc32)nc2c(Nc3cccc4ccccc34)cccc21 ; CCCCn1c(-n2c3ccc(C(C)(C)C)cc3c3cc(C(C)(C)C)ccc32)nc2c(NC[Si](C)(C)C)cccc21 ; CCCCn1c(-n2c3ccc(C(C)(C)C)cc3c3cc(C(C)(C)C)ccc32)nc2c(Nc3ccccc3C(C)C)cccc21 ; CCCCn1c(-n2c3ccc(C(C)(C)C)cc3c3cc(C(C)(C)C)ccc32)nc2c(NCC(C)(C)C)cccc21]

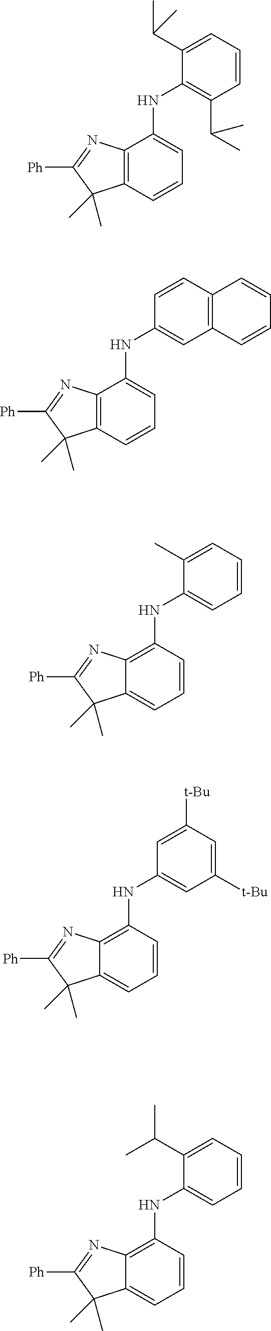 [SMILES: CC(C)c1cccc(C(C)C)c1Nc1cccc2c1N=C(c1ccccc1)C2(C)C ; CC1(C)C(c2ccccc2)=Nc2c(Nc3ccc4ccccc4c3)cccc21 ; Cc1ccccc1Nc1cccc2c1N=C(c1ccccc1)C2(C)C ; CC(C)(C)c1cc(Nc2cccc3c2N=C(c2ccccc2)C3(C)C)cc(C(C)(C)C)c1 ; CC(C)c1ccccc1Nc1cccc2c1N=C(c1ccccc1)C2(C)C]

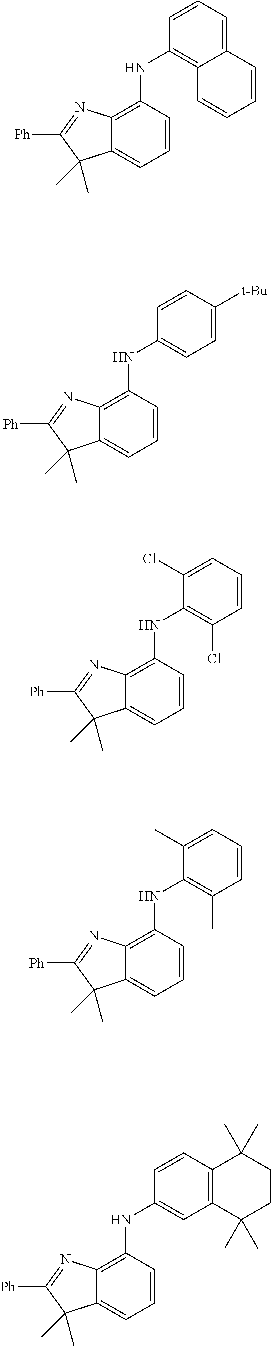 [SMILES: CC1(C)C(c2ccccc2)=Nc2c(Nc3cccc4ccccc34)cccc21 ; CC(C)(C)c1ccc(Nc2cccc3c2N=C(c2ccccc2)C3(C)C)cc1 ; CC1(C)C(c2ccccc2)=Nc2c(Nc3c(Cl)cccc3Cl)cccc21 ; Cc1cccc(C)c1Nc1cccc2c1N=C(c1ccccc1)C2(C)C ; CC1(C)CCC(C)(C)c2cc(Nc3cccc4c3N=C(c3ccccc3)C4(C)C)ccc21]

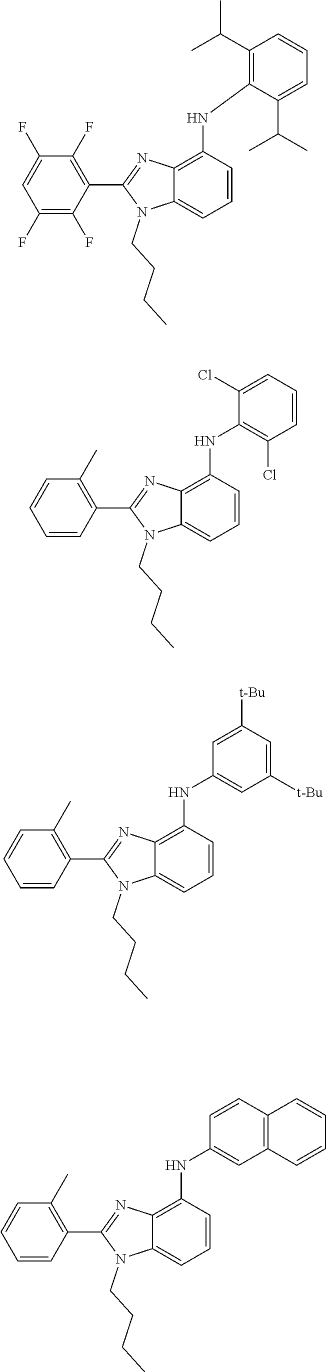 [SMILES: CCCCn1c(-c2c(F)c(F)cc(F)c2F)nc2c(Nc3c(C(C)C)cccc3C(C)C)cccc21 ; CCCCn1c(-c2ccccc2C)nc2c(Nc3c(Cl)cccc3Cl)cccc21 ; CCCCn1c(-c2ccccc2C)nc2c(Nc3cc(C(C)(C)C)cc(C(C)(C)C)c3)cccc21 ; CCCCn1c(-c2ccccc2C)nc2c(Nc3ccc4ccccc4c3)cccc21]

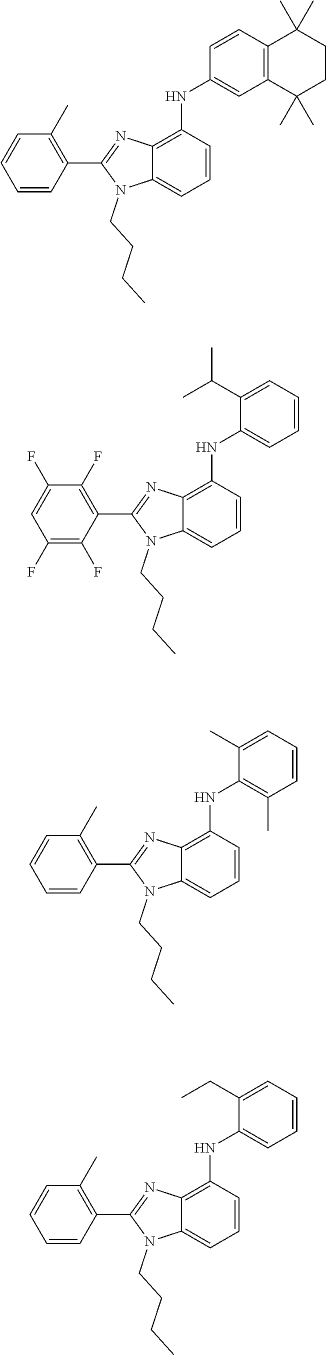 [SMILES: CCCCn1c(-c2ccccc2C)nc2c(Nc3ccc4c(c3)C(C)(C)CCC4(C)C)cccc21 ; CCCCn1c(-c2c(F)c(F)cc(F)c2F)nc2c(Nc3ccccc3C(C)C)cccc21 ; CCCCn1c(-c2ccccc2C)nc2c(Nc3c(C)cccc3C)cccc21 ; CCCCn1c(-c2ccccc2C)nc2c(Nc3ccccc3CC)cccc21]

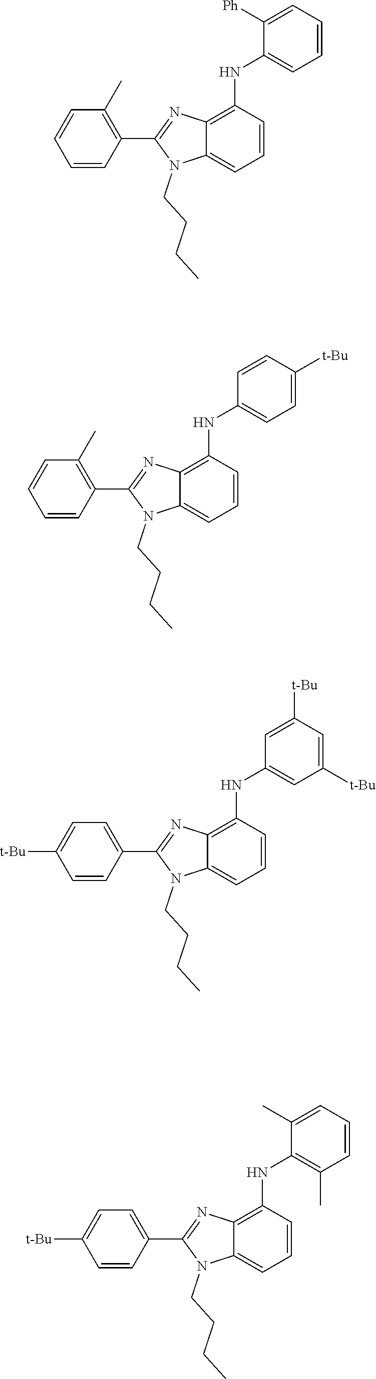 [SMILES: CCCCn1c(-c2ccccc2C)nc2c(Nc3ccccc3-c3ccccc3)cccc21 ; CCCCn1c(-c2ccccc2C)nc2c(Nc3ccc(C(C)(C)C)cc3)cccc21 ; CCCCn1c(-c2ccc(C(C)(C)C)cc2)nc2c(Nc3cc(C(C)(C)C)cc(C(C)(C)C)c3)cccc21 ; CCCCn1c(-c2ccc(C(C)(C)C)cc2)nc2c(Nc3c(C)cccc3C)cccc21]

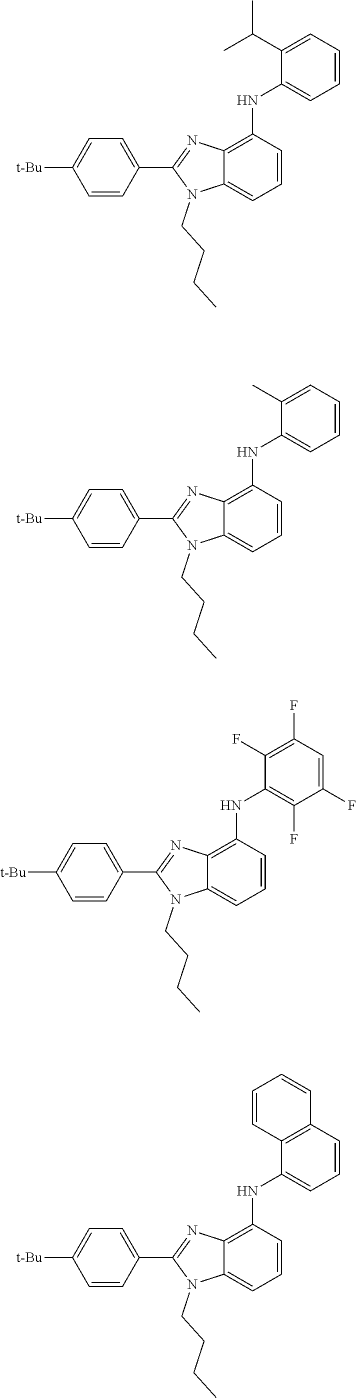 [SMILES: CCCCn1c(-c2ccc(C(C)(C)C)cc2)nc2c(Nc3ccccc3C(C)C)cccc21 ; CCCCn1c(-c2ccc(C(C)(C)C)cc2)nc2c(Nc3ccccc3C)cccc21 ; CCCCn1c(-c2ccc(C(C)(C)C)cc2)nc2c(Nc3c(F)c(F)cc(F)c3F)cccc21 ; CCCCn1c(-c2ccc(C(C)(C)C)cc2)nc2c(Nc3cccc4ccccc34)cccc21]

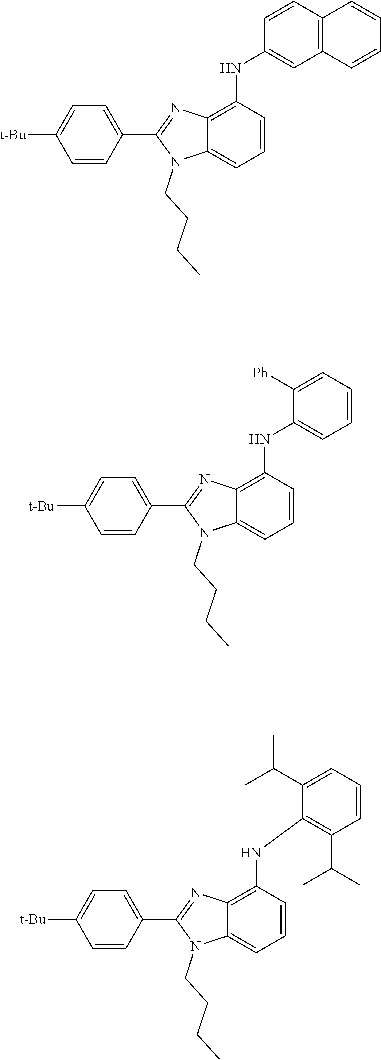 [SMILES: CCCCn1c(-c2ccc(C(C)(C)C)cc2)nc2c(Nc3ccc4ccccc4c3)cccc21 ; CCCCn1c(-c2ccc(C(C)(C)C)cc2)nc2c(Nc3ccccc3-c3ccccc3)cccc21 ; CCCCn1c(-c2ccc(C(C)(C)C)cc2)nc2c(Nc3c(C(C)C)cccc3C(C)C)cccc21]

Embodiments of this disclosure includes polymerization processes. The polymerization processes include polymerizing ethylene and one or more olefins in the presence of a catalyst system under olefin polymerization conditions to form an ethylene-based polymer, the catalyst system comprising a metal-ligand complex according to formula (I) or formula (II).

One or more embodiments of this disclosure include processes for polymerizing polymers, the process comprising: contacting ethylene and optionally one or more (C₃-C₁₂)α-olefins in the presence of a catalyst system in a reactor. The catalyst system may include procatalyst according to the metal-ligand complex of formula (I) and an activator. The polymerization processes may include, but are not limited to, solution polymerization process, gas phase polymerization process, slurry phase polymerization process, and combinations thereof using one or more reactors such as loop reactors, isothermal reactors, fluidized bed gas phase reactors, continuous stirred tank reactors, batch reactors in parallel, series, and/or any combinations thereof.

The polymerization process of this disclosure may produce ethylene based polymers, for example homopolymers and/or interpolymers (including copolymers) of ethylene and optionally one or more comonomers such as a-olefins may, for example, be produced via solution-phase polymerization process using one or more loop reactors, isothermal reactors, and combinations thereof.

In some embodiments, the solution phase polymerization process occurs in one or more well-stirred reactors such as one or more loop reactors or one or more spherical isothermal reactors at a temperature in the range of from 120 to 300° C.; for example, from 150 to 190° C. and at pressures in the range of from 300 to 1500 psi; for example, from 400 to 750 psi. The residence time in solution phase polymerization process is typically in the range of from 2 to 30 minutes: for example, from 10 to 20 minutes. Ethylene, one or more solvents, one or more catalyst systems, such as catalyst system that includes a procatalyst according to the metal-ligand complex of formula (I), optionally one or more cocatalysts, and optionally one or more comonomers are fed continuously to the one or more reactors. Exemplary solvents include, but are not limited to, isoparaffins. For example, such solvents are commercially available under the name ISOPAR E from ExxonMobil Chemical Co., Houston, Texas. The resultant mixture of the ethylene based polymer and solvent is then removed from the reactor and the ethylene based polymer is isolated. Solvent is typically recovered via a solvent recovery unit. i.e. heat exchangers and vapor liquid separator drum, and is then recycled back into the polymerization system.

Chain Shuttling and/or Chain Transfer Agent

In one or more embodiments, the polymerization processes of this disclosure include contacting ethylene and/or one or more (C₃-C₁₂)α-olefins in a reactor in the presence of a catalyst system and a chain transfer agent or chain shuttling agent. In such embodiments, the polymerization process includes three components: (A) a procatalyst comprising a metal-ligand complex having a structure of formula (I) and, optionally, a cocatalyst; (B) an olefin polymerization catalyst having a comonomer selectivity different from that of the procatalyst (A); and (C) the chain transfer agent or chain shuttling agent.

As additions to a catalyst system, chain transfer agents and chain shuttling agents are compounds capable of transferring polymer chains between two catalyst molecules in a single polymerization reactor. The catalyst molecules may have the same structure or different structures. When the catalyst molecules have different structures, they may have different monomer selectivities. Whether the compounds function as chain transfer agents or as chain shuttling agents depends on the type of polymerization reactor, even though the three components (A)-(C) previously described may be chemically identical in either type of polymerization reactor. For example, in a batch reactor with a single-catalyst system or a dual-catalyst system, the compounds function as chain transfer agents. In a continuous reactor with a dual-catalyst system, the compounds function as chain shuttling agents. In general, compounds that function as chain transfer agents in a batch reactor also can function as chain shuttling agents in a continuous reactor; conversely, molecules that function as chain shuttling agents also can function as chain transfer agents. Therefore, in embodiments of polymerization processes in this disclosure, it should be understood that disclosure of a compound as a “chain transfer agent” further constitutes disclosure of the same compounds as a “chain shuttling agent.” Thus, the terms “chain transfer agent” and “chain shuttling agent” are interchangeable with respect to chemical compounds but are distinguishable when a process is specified to occur within a particular kind of polymerization reactor.

A catalyst's chain transfer ability is initially evaluated by running a campaign in which the level of chain transfer or shuttling agent (CSA) is varied to observe the depression in molecular weight and overall effect on the PDI expected for a shuttling catalyst. The molecular weight of the polymer generated by catalysts with potential to be good chain shutters will be more sensitive to the addition of CSA than the polymer molecular weight generated by catalysts exhibiting poorer shuttling or slower chain transfers kinetics. The Mayo equation (Equation 1) describes how a chain transfer agent decreases the number average chain length (X̅ₙ̅) from the native number average chain length (X̅ₙ̅₀̅) where no chain transfer agent is present. Equation 2 defines a chain transfer or chain shuttling constant, Ca, as the ratio of chain transfer and propagation rate constants. By assuming that the vast majority of chain propagation occurs through ethylene insertion and not comonomer incorporation, Equation 3 describes the expected Mn of a polymerization. Mn₀ is the native molecular weight of the catalyst in the absence of chain shuttling agent and Mn is the molecular weight that is observed with chain shuttling agent (Mn=Mn₀ with no chain shuttling agent).

$$\frac{1}{{\overline{X}}_{n}} = \frac{1}{{\overline{X}}_{{n}_{0}}} + \frac{{k}_{tr} [ chain transfer agent ]}{{k}_{p} [ monomer ]} \qquad Equation 1$$

$$Ca = \frac{{k}_{tr}}{{k}_{p}} \qquad Equation 2$$

$$\frac{1}{Mn} = \frac{1}{{Mn}_{0}} + Ca \frac{[ CSA ]}{[ monomer ] \times 28} \qquad Equation 3$$

$$[ Monomer ] = ( Mol \% C 2 ) \times [ ethylene ] + ( Mol \% C 8 ) \times [ octene ] \qquad Equation 4$$

Typically, chain transfer agents comprise a metal that is Al, B, or Ga being in a formal oxidation state of +3; or a metal that is Zn or Mg being in a formal oxidation state of +2. Chain transfer agents suitable for processes of this disclosure are described in U.S. Patent Application Publication Number US 2007/0167315, which is incorporated herein by reference in its entirety.

In one or more embodiments of the polymerization process, the chain transfer agent, when present, may be chosen from diethylzinc, di(iso-butyl)zinc, di(n-hexyl)zinc, di(n-octyl)zinc, triethylaluminum, trioctylaluminum, triethylgallium, iso-butylaluminum bis(dimethyl(t-butyl)siloxane), iso-butylaluminum bis(di(trimethylsilyl)amide), n-octylaluminum di(pyridine-2-methoxide), bis(n-octadecyl) iso-butylaluminum, iso-butylaluminum bis(di(n-pentyl) amide), n-octylaluminum bis(2,6-di-t-butylphenoxide, n-octylaluminum di(ethyl(1-naphthyl) amide), ethylaluminum bis(t-butyldimethylsiloxide), ethylaluminum di(bis(trimethylsilyl)amide), ethylaluminum bis(2,3,6,7-dibenzo-1-azacycloheptaneamide), n-octylaluminum bis(2,3,6,7-dibenzo-1-azacycloheptaneamide), n-octylaluminum bis(dimethyl(t-butyl)siloxide, ethylzinc (2,6-diphenylphenoxide), ethylzinc (t-butoxide), dimethylmagnesium, dibutylmagnesium, and n-butyl-sec-butylmagnesium.

Cocatalyst Component

The catalyst system comprising a metal-ligand complex of formula (I) may be rendered catalytically active by any technique known in the art for activating metal-based catalysts of olefin polymerization reactions. For example, the procatalyst according to a metal-ligand complex of formula (I) may be rendered catalytically active by contacting the complex to, or combining the complex with, an activating co-catalyst. Additionally, the metal-ligand complex according for formula (I) includes both a procatalyst form, which is neutral, and a catalytic form, which may be positively charged due to the loss of a monoanionic ligand, such a benzyl or phenyl. Suitable activating co-catalysts for use herein include alkyl aluminums; polymeric or oligomeric alumoxanes (also known as aluminoxanes); neutral Lewis acids; and non-polymeric, non-coordinating, ion-forming compounds (including the use of such compounds under oxidizing conditions). A suitable activating technique is bulk electrolysis. Combinations of one or more of the foregoing activating co-catalysts and techniques are also contemplated. The term “alkyl aluminum” means a monoalkyl aluminum dihydride or monoalkylaluminum dihalide, a dialkyl aluminum hydride or dialkyl aluminum halide, or a trialkylaluminum. Examples of polymeric or oligomeric alumoxanes include methylalumoxane, triisobutylaluminum-modified methylalumoxane, and isobutylalumoxane.

In some embodiments, suitable cocatalysts for use include polymeric or oligomeric aluminoxanes, especially methyl aluminoxane, as well as inert, compatible, noncoordinating, ion forming compounds. Exemplary suitable cocatalysts include, but are not limited to modified methyl aluminoxane (MMAO), bis(hydrogenated tallow alkyl)methyl, tetrakis(pentafluorophenyl)borate(1-) amine (RIBS-2), triethyl aluminum (TEA), and combinations thereof.

Lewis acid activating co-catalysts include Group 13 metal compounds containing (C₁-C₂₀)hydrocarbyl substituents as described herein. In some embodiments. Group 13 metal compounds are tri((C₁-C₂₀)hydrocarbyl)-substituted-aluminum or tri((C₁-C₂₀)hydrocarbyl)-boron compounds. In other embodiments. Group 13 metal compounds are tri(hydrocarbyl)-substituted-aluminum, tri((C₁-C₂₀)hydrocarbyl)-boron compounds, tri((C₁-C₁₀)alkyl)aluminum, tri((C₆-C₁₅)aryl)boron compounds, and halogenated (including perhalogenated) derivatives thereof. In further embodiments, Group 13 metal compounds are tris(fluoro-substituted phenyl)boranes, tris(pentafluorophenyl)borane. In some embodiments, the activating co-catalyst is a tris((C₁-C₂₀)hydrocarbyl borate (e.g. trityl tetrafluoroborate) or a tri((C₁-C₂₀)hydrocarbyl)ammonium tetra((C₁-C₂₀)hydrocarbyl)borane (e.g. bis(octadecyl)methylammonium tetrakis(pentafluorophenyl)borane). As used herein, the term “ammonium” means a nitrogen cation that is a ((C₁-C₂₀)hydrocarbyl)₄N⁺ a ((C₁-C₂₀)hydrocarbyl)₃N(H)⁺, a ((C₁-C₂₀)hydrocarbyl)₂N(H)₂⁺, (C₁-C₂₀)hydrocarbylN(H)₃⁺, or N(H)₄⁺, wherein each (C₁-C₂₀)hydrocarbyl, when two or more are present, may be the same or different.

Combinations of neutral Lewis acid activating co-catalysts include mixtures comprising a combination of a tri((C₁-C₄)alkyl)aluminum and a halogenated tri((C₆-C₁₈)aryl)boron compound, especially a tris(pentafluorophenyl)borane. Other embodiments are combinations of such neutral Lewis acid mixtures with a polymeric or oligomeric alumoxane, and combinations of a single neutral Lewis acid, especially tris(pentafluorophenyl)borane with a polymeric or oligomeric alumoxane. Ratios of numbers of moles of (metal-ligand complex): (tris(pentafluoro-phenylborane): (alumoxane) [e.g., (Group 4 metal-ligand complex):(tris(pentafluoro-phenylborane):(alumoxane)] are from 1:1:1 to 1:10:30, in other embodiments, from 1:1:1.5 to 1:5:10.

The catalyst system that includes the metal-ligand complex of formula (I) may be activated to form an active catalyst composition by combination with one or more cocatalysts, for example, a cation forming cocatalyst, a strong Lewis acid, or combinations thereof. Suitable activating co-catalysts include polymeric or oligomeric aluminoxanes, especially methyl aluminoxane, as well as inert, compatible, noncoordinating, ion forming compounds. Exemplary suitable co-catalysts include, but are not limited to modified methyl aluminoxane (MMAO), bis(hydrogenated tallow alkyl)methyl, tetrakis(pentafluorophenyl)borate(1-) amine, and combinations thereof.

In some embodiments, more than one of the foregoing activating co-catalysts may be used in combination with each other. A specific example of a co-catalyst combination is a mixture of a tri((C₁-C₄)hydrocarbyl)aluminum, tri((C₁-C₄)hydrocarbyl)borane, or an ammonium borate with an oligomeric or polymeric alumoxane compound. The ratio of total number of moles of one or more metal-ligand complexes of formula (I) to total number of moles of one or more of the activating co-catalysts is from 1:10.000 to 100:1. In some embodiments, the ratio is at least 1:5000, in some other embodiments, at least 1:1000; and 10:1 or less, and in some other embodiments, 1:1 or less. When an alumoxane alone is used as the activating co-catalyst, preferably the number of moles of the alumoxane that are employed is at least 100 times the number of moles of the metal-ligand complex of formula (I). When tris(pentafluorophenyl)borane alone is used as the activating co-catalyst, in some other embodiments, the number of moles of the tris(pentafluorophenyl)borane that are employed to the total number of moles of one or more metal-ligand complexes of formula (I) from 0.5:1 to 10:1, from 1:1 to 6:1, or from 1:1 to 5:1. The remaining activating co-catalysts are generally employed in approximately mole quantities equal to the total mole quantities of one or more metal-ligand complexes of formula (I).

### Polyolefins

The catalytic systems described in the preceding paragraphs are utilized in the polymerization of olefins, primarily ethylene and propylene. In some embodiments, there is only a single type of olefin or α-olefin in the polymerization scheme, creating a homopolymer. However, additional α-olefins may be incorporated into the polymerization procedure. The additional α-olefin co-monomers typically have no more than 20 carbon atoms. For example, the α-olefin co-monomers may have 3 to 10 carbon atoms or 3 to 8 carbon atoms. Exemplary α-olefin co-monomers include, but are not limited to, propylene, 1-butene, 1-pentene, 1-hexene, 1-heptene, 1-octene, 1-nonene, 1-decene, and 4-methyl-1-pentene. For example, the one or more α-olefin co-monomers may be selected from the group consisting of propylene, 1-butene, 1-hexene, and 1-octene; or in the alternative, from the group consisting of 1-hexene and 1-octene.

The ethylene based polymers, for example homopolymers and/or interpolymers (including copolymers) of ethylene and optionally one or more co-monomers such as α-olefins, may comprise from at least 50 percent by weight monomer units derived from ethylene. All individual values and subranges encompassed by “from at least 50 weight percent” are disclosed herein as separate embodiments; for example, the ethylene based polymers, homopolymers and/or interpolymers (including copolymers) of ethylene and optionally one or more co-monomers such as α-olefins may comprise at least 60 weight percent monomer units derived from ethylene; at least 70 weight percent monomer units derived from ethylene; at least 80 weight percent monomer units derived from ethylene; or from 50 to 100 weight percent monomer units derived from ethylene; or from 80 to 100 weight percent units derived from ethylene.

In some embodiments, the ethylene based polymers may comprise at least 90 mole percent units derived from ethylene. All individual values and subranges from at least 90 mole percent are included herein and disclosed herein as separate embodiments. For example, the ethylene based polymers may comprise at least 93 mole percent units derived from ethylene; at least 96 mole percent units; at least 97 mole percent units derived from ethylene; or in the alternative, from 90 to 100 mole percent units derived from ethylene; from 90 to 99.5 mole percent units derived from ethylene; or from 97 to 99.5 mole percent units derived from ethylene.

In some embodiments of the ethylene based polymer, the amount of additional α-olefin is less than 50%; other embodiments include at least 0.5 mole percent (mol %) to 25 mol %; and in further embodiments the amount of additional α-olefin includes at least 5 mol % to 10 mol %. In some embodiments, the additional α-olefin is 1-octene.

Any conventional polymerization processes may be employed to produce the ethylene based polymers. Such conventional polymerization processes include, but are not limited to, solution polymerization processes, gas phase polymerization processes, slurry phase polymerization processes, and combinations thereof using one or more conventional reactors such as loop reactors, isothermal reactors, fluidized bed gas phase reactors, stirred tank reactors, batch reactors in parallel, series, or any combinations thereof, for example.

In one embodiment, the ethylene based polymer may be produced via solution polymerization in a dual reactor system, for example a dual loop reactor system, wherein ethylene and optionally one or more α-olefins are polymerized in the presence of the catalyst system, as described herein, and optionally one or more co-catalysts. In another embodiment, the ethylene based polymer may be produced via solution polymerization in a dual reactor system, for example a dual loop reactor system, wherein ethylene and optionally one or more α-olefins are polymerized in the presence of the catalyst system in this disclosure, and as described herein, and optionally one or more other catalysts. The catalyst system, as described herein, can be used in the first reactor, or second reactor, optionally in combination with one or more other catalysts. In one embodiment, the ethylene based polymer may be produced via solution polymerization in a dual reactor system, for example a dual loop reactor system, wherein ethylene and optionally one or more α-olefins are polymerized in the presence of the catalyst system, as described herein, in both reactors.

In another embodiment, the ethylene based polymer may be produced via solution polymerization in a single reactor system, for example a single loop reactor system, in which ethylene and optionally one or more α-olefins are polymerized in the presence of the catalyst system, as described within this disclosure, and optionally one or more cocatalysts, as described in the preceding paragraphs.

The ethylene based polymers may further comprise one or more additives. Such additives include, but are not limited to, antistatic agents, color enhancers, dyes, lubricants, pigments, primary antioxidants, secondary antioxidants, processing aids, UV stabilizers, and combinations thereof. The ethylene based polymers may contain any amounts of additives. The ethylene based polymers may compromise from about 0 to about 10 percent by the combined weight of such additives, based on the weight of the ethylene based polymers and the one or more additives. The ethylene based polymers may further comprise fillers, which may include, but are not limited to, organic or inorganic fillers. The ethylene based polymers may contain from about 0 to about 20 weight percent fillers such as, for example, calcium carbonate, talc, or Mg(OH)₂, based on the combined weight of the ethylene based polymers and all additives or fillers. The ethylene based polymers may further be blended with one or more polymers to form a blend.

In some embodiments, a polymerization process for producing an ethylene-based polymer may include polymerizing ethylene and at least one additional α-olefin in the presence of a catalyst system, wherein the catalyst system incorporates at least one metal-ligand complex of formula (I). The polymer resulting from such a catalyst system that incorporates the metal-ligand complex of formula (I) may have a density according to ASTM D792 (incorporated herein by reference in its entirety) from 0.850 g/cm³ to 0.950 g/cm³, from 0.880 g/cm³ to 0.920 g/cm³, from 0.880 g/cm³ to 0.910 g/cm³, or from 0.880 g/cm³ to 0.900 g/cm³, for example.

In another embodiment, the polymer resulting from the catalyst system that includes the metal-ligand complex of formula (I) has a melt flow ratio (I₁₀/I₂) from 5 to 15, in which melt index 12 is measured according to ASTM D1238 (incorporated herein by reference in its entirety) at 190° C., and 2.16 kg load, and melt index I₁₀ is measured according to ASTM D1238 at 190° C. and 10 kg load. In other embodiments the melt flow ratio (I₁₀/I₂) is from 5 to 10, and in others, the melt flow ratio is from 5 to 9.

In some embodiments, the polymer resulting from the catalyst system that includes the metal-ligand complex of formula (I) has a molecular-weight distribution (MWD) from 1 to 25, where MWD is defined as Mw/Mn with M, being a weight-average molecular weight and Mₙ being a number-average molecular weight. In other embodiments, the polymers resulting from the catalyst system have a MWD from 1 to 6. Another embodiment includes a MWD from 1 to 3; and other embodiments include MWD from 1.5 to 2.5.

Embodiments of the catalyst systems described in this disclosure yield unique polymer properties as a result of the high molecular weights of the polymers formed and the amount of the co-monomers incorporated into the polymers.

All solvents and reagents are obtained from commercial sources and used as received unless otherwise noted. Anhydrous toluene, hexanes, tetrahydrofuran, and diethyl ether are purified via passage through activated alumina and, in some cases, Q-5 reactant. Solvents used for experiments performed in a nitrogen-filled glovebox are further dried by storage over activated 4 Å molecular sieves. Glassware for moisture-sensitive reactions is dried in an oven overnight prior to use. NMR spectra are recorded on Varian 400-MR and VNMRS-500 spectrometers. LC-MS analyses are performed using a Waters e2695 Separations Module coupled with a Waters 2424 ELS detector, a Waters 2998 PDA detector, and a Waters 3100 ESI mass detector. LC-MS separations are performed on an XBridge C18 3.5 μm 2.1×50 mm column using a 5:95 to 100:0 acetonitrile to water gradient with 0.1% formic acid as the ionizing agent. HRMS analyses are performed using an Agilent 1290 Infinity LC with a Zorbax Eclipse Plus C18 1.8 μm 2.1×50 mm column coupled with an Agilent 6230 TOF Mass Spectrometer with electrospray ionization. ¹H NMR data are reported as follows: chemical shift (multiplicity (br=broad, s=singlet, d=doublet, t=triplet, q=quartet, p=pentet, sex=sextet, sept=septet and m=multiplet), integration, and assignment). Chemical shifts for ¹H NMR data are reported in ppm downfield from internal tetramethylsilane (TMS, δ scale) using residual protons in the deuterated solvent as references. ¹³C NMR data are determined with ¹H decoupling, and the chemical shifts are reported downfield from tetramethylsilane (TMS, δ scale) in ppm versus the using residual carbons in the deuterated solvent as references.

### High-Throughput Parallel Polymerization Reactor Polymerization Procedure (PPR) Screening

Polyolefin catalysis screening is performed in a high-throughput parallel polymerization reactor (PPR) system. The PPR system comprises an array of 48 single-cell (6×8 matrix) reactors in an inert atmosphere glovebox. Each cell is equipped with a glass insert with an internal working liquid volume of approximately 5 mL. Each cell has independent controls for pressure and is continuously stirred at 800 rpm. Catalyst solutions, unless otherwise noted, are prepared in toluene. All liquids (i.e., solvent, 1-octene, chain shuttling agent solutions, and catalyst solutions) were added via robotic syringes. Gaseous reagents (i.e., ethylene, CO) were added via a gas injection port. Prior to each run, the reactors were heated to 80° C., purged with ethylene, and vented.

The reactors are heated to the run temperature and then pressurized to the appropriate psig with ethylene. Isopar E waiss added, and then toluene solutions of reagents are added in the following order: (1) 1-octene with 500 nmol of scavenger MMAO-3A; (2) Activator (RIBS-II, FAB, etc.); and (3) Catalyst (100 nmol).

Each liquid addition is chased with a small amount of Isopar E so that after the final addition a total reaction volume of 5 mL is reached. Upon addition of the catalyst, the PPR software began monitoring the pressure of each cell. The desired pressure (within approximately 2-6 psig) was maintained by the supplemental addition of ethylene gas by opening the valve at the set point minus 1 psi and closing it when the pressure reaches 2 psi higher. All drops in pressure are cumulatively recorded as “Uptake” or “Conversion” of the ethylene for the duration of the run or until the uptake or conversion requested value is reached, whichever occurred first. Each reaction is then quenched by addition of 10% carbon monoxide in argon for 4 minutes at 40-50 psi higher than the reactor pressure. The shorter the “Quench Time”, the more active the catalyst. In order to prevent the formation of too much polymer in any given cell, the reaction is quenched upon reaching a predetermined uptake level (50 psig for 120° C. runs, 75 psig for 150° C. runs). After the reactors were quenched, they were allowed to cool to 70° C., vented, purged for 5 minutes with nitrogen to remove carbon monoxide, and the tubes were removed. The polymer samples are then dried in a centrifugal evaporator at 70° C. for 12 hours, weighed to determine polymer yield, and submitted for IR (1-octene incorporation) and GPC (molecular weight) analysis.

### Batch Reactor Polymerization Procedure

The batch reactor polymerizations are conducted in a 2-L Parr™ batch reactor. The reactor is heated by an electrical heating mantle, and is cooled by an internal serpentine cooling coil containing cooling water. Both the reactor and the heating/cooling system are controlled and monitored by a Camile™ TG process computer. The bottom of the reactor is fitted with a dump valve, which empties the reactor contents into a stainless-steel dump pot, which is prefilled with a catalyst kill solution (typically 5 mL of an Irgafos/Irganox/toluene mixture). The dump pot is vented to a 30-gal, blow-down tank, with both the pot and the tank purged with nitrogen. All solvents used for polymerization or catalyst makeup are run through solvent purification columns to remove any impurities that may affect polymerization. The 1-octene and Isopar E are passed through two columns, the first containing activated A2 alumina, the second containing activated Q5 reactant. The ethylene was passed through two columns, the first containing A204 alumina and 4 Å mol sieves, the second containing Q5 reactant. The N₂, used for transfers, is passed through a single column containing A204 alumna, 4 Å mol sieves and Q5.

The reactor is loaded first from the shot tank that contains Isopar E solvent and/or 1-octene, depending on desired reactor loading. The shot tank is filled to the load set points by use of a lab scale to which the shot tank is mounted. After liquid feed addition, the reactor is heated up to the polymerization temperature set point. If ethylene is used, it is added to the reactor when at reaction temperature to maintain reaction pressure set point. Ethylene addition amounts are monitored by a micro-motion flow meter.

The catalyst and activators were mixed with the appropriate amount of purified toluene to achieve a solution of the desired molarity. The catalyst and activators were handled in an inert glove box, drawn into a syringe and pressure transferred into the catalyst shot tank. This was followed by three rinses of toluene, 5-mL each. Immediately after catalyst addition the run timer began. If ethylene was used, it was then added by the Camile to maintain reaction the pressure set point in the reactor. These polymerizations are run for 10 min., then the agitator is stopped and the bottom dump valve is opened to empty reactor contents into the dump pot. The dump pot contents are poured into trays placed in a lab hood where the solvent was evaporated off overnight. The trays containing the remaining polymer were then transferred to a vacuum oven, where they were heated up to 140° C. under vacuum to remove any remaining solvent. After the trays cooled to ambient temperature, the polymers were weighed for yield/efficiencies, and submitted for polymer testing.

HT-GPC Analysis with IR Detection of Octene Incorporation

High-temperature GPC analysis was performed using a Dow Robot Assisted Delivery (RAD) system equipped with a PolymerChar infrared detector (IR5) and Agilent PLgel Mixed A columns. Decane (10 μL) was added to each sample for use as an internal flow marker. Samples were first diluted in 1,2,4-trichlorobenzene (TCB) stabilized with 300 ppm of butylated hydroxytoluene (BHT) to a concentration of 10 mg/mL and dissolved by stirring at 160° C. for 120 minutes. Prior to injection samples were further diluted with TCB stabilized with BHT to a concentration of 2 mg/mL. Samples (250 μL) were eluted through one PL-gel 20 μm (50×7.5 mm) guard column followed by two PL-gel 20 μm (300×7.5 mm) Mixed-A columns maintained at 160° C. with TCB stabilized with BHT at a flowrate of 1.0 mL/min. The total run time was 24 minutes. To calibrate for molecular weight Agilent EasiCal polystyrene standards (PS-1 and PS-2) were diluted with 1.5 mL of TCB stabilized with BHT and dissolved by stirring at 160° C. for 15 minutes. The PS standards were injected into the system without further dilution to create a 3^{rd}-order MW calibration curve with apparent units adjusted to homo-polyethylene (PE) using known Mark-Houwink coefficients for PS and PE. Octene incorporation was determined by use of a linear calibration developed by analyzing copolymers of known compositions.

### SymRAD HT-GPC Analysis

The molecular weight data is determined by analysis on a hybrid Symyx/Dow built Robot-Assisted Dilution High-Temperature Gel Permeation Chromatographer (Sym-RAD-GPC). The polymer samples are dissolved by heating for 120 minutes at 160° C. in 1,2,4-trichlorobenzene (TCB) at a concentration of 10 mg/mL stabilized by 300 parts per million (ppm) of butylated hydroxyl toluene (BHT). Each sample was diluted to 1 mg/mL immediately before the injection of a 250 μL aliquot of the sample. The GPC is equipped with two Polymer Labs PLgel 10 μm MIXED-B columns (300×10 mm) at a flow rate of 2.0 mL/minute at 160° C. Sample detection is performed using a PolyChar IR4 detector in concentration mode. A conventional calibration of narrow polystyrene (PS) standards is utilized with apparent units adjusted to homo-polyethylene (PE) using known Mark-Houwink coefficients for PS and PE in TCB at this temperature.

### 1-Octene Incorporation IR Analysis

The running of samples for the HT-GPC analysis precedes the IR analysis. For the IR analysis, a 48-well HT silicon wafer is utilized for deposition and analysis of 1-octene incorporation of samples. For the analysis, the samples are heated to 160° C. for less than or equal to 210 minutes; the samples are reheated to remove magnetic GPC stir bars and are shaken with glass-rod stir bars on a J-KEM Scientific heated robotic shaker. Samples are deposited while being heated using a Tecan MiniPrep 75 deposition station, and the 1,2,4-trichlorobenzene is evaporated off the deposited wells of the wafer at 160° C. under nitrogen purge. The analysis of 1-octene is performed on the HT silicon wafer using a NEXUS 670 E.S.P. FT-IR.

### EXAMPLES

Examples 1 to 90 are synthetic procedures for ligand intermediates, ligands, and isolated procatalysts Structures of Ligands 1-43. Inventive Metal-ligand Complex 1 to Inventive Metal-ligand Complex 13 (IMLC-1 to IMLC-13) were synthesized from various Ligands 1-43. In Examples 91 and 92, the results of the polymerization reactions of IMLC-1 to IMLC-13 and metal-ligand complexes produced in situ are tabulated and discussed. One or more features of the present disclosure are illustrated in view of the examples as follows:

### Synthesis of Metal-Ligand Complexes

### Example 1—Synthesis of Synthesis of N¹-Hexyl-3-Nitrobenzene-1,2-Diamine

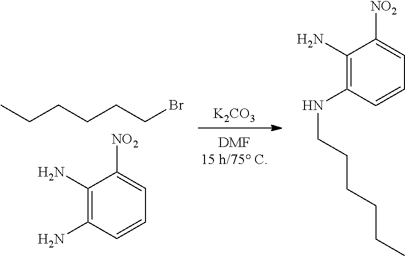

A 250-mL round-bottom flask was charged with 3-nitrobenzene-1,2-diamine (5.00 g, 32.65 mmol), K₂CO₃ (9.02 g, 65.30 mmol), and DMF (80 mL). 1-Bromohexane (4.6 mL, 32.65 mmol) was added and allowed to stir under nitrogen for 15 h at 75° C. Water and EtOAc were added and the organic layer was collected and washed multiple times with brine. All volatiles were removed and the crude product was purified by column chromatography (100% hexanes gradient to 100% EtOAc). Some impurities remained, but were carried on to the next step. Yield: 7.75 g, 71%.

¹H NMR (400 MHz, CDCl₃) δ 7.68 (dd, J=8.7, 1.3 Hz, 1H), 6.86 (dd, J=7.7, 1.4 Hz, 1H), 6.72 (dd, J=8.7, 7.6 Hz, 1H), 5.97 (s, 2H), 3.11 (t, J=7.1 Hz, 2H), 1.71 (dq, J=15.7, 7.2, 6.6 Hz, 2H), 1.54-1.42 (m, 2H), 1.42-1.30 (m, 6H), 0.96-0.91 (m, 3H).

¹³C NMR (101 MHz, CDCl₃) δ 138.41, 136.35, 133.28, 117.07, 117.05, 115.98, 45.00, 31.64, 29.52, 26.93, 22.62, 14.04.

### Example 2—Synthesis of 2-(3,5-di-tert-butylphenyl)-1-hexyl-4-nitro-1H-benzo[d]imidazole

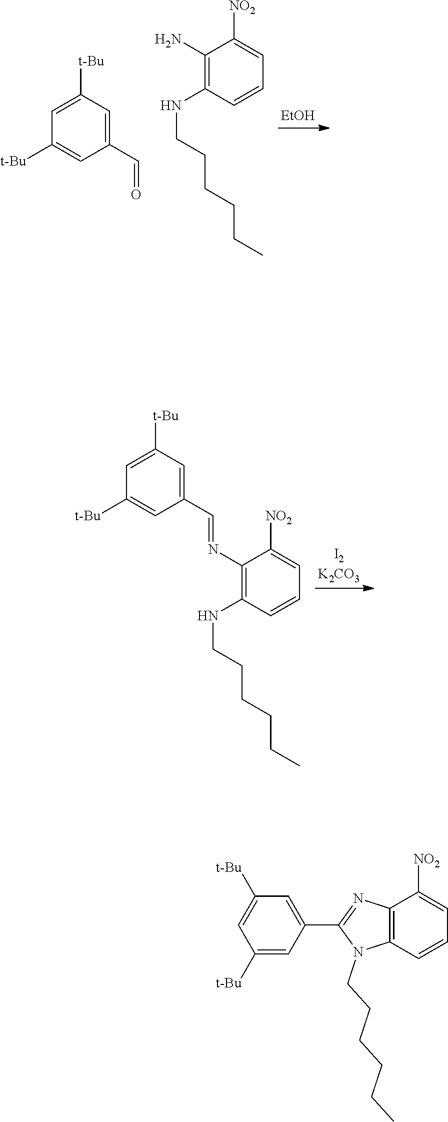

A 20-mL vial was charged with the N¹-hexyl-3-nitrobenzene-1,2-diamine (0.263 g, 1.11 mmol), 3,5-di-tert-butylbenzaldehyde (0.242 g, 1.11 mmol), and EtOH (7 mL). The solution was heated overnight at 75° C. All volatiles were removed, then K₂CO₃ (0.337 g, 2.44 mmol) and CH₂Cl₂ (8 mL) were added, followed by iodine (0.281 g, 1.11 mmol). The reaction was stirred for 2 h at room temperature. Water was added and the organic layer was extracted (solvent?). All volatiles were removed and the crude product was purified by column chromatography (Hex:EtOAc, 70:30). Yield: 0.483 g, 54%.

¹H NMR (400 MHz, CDCl₃) δ 8.18 (dd, J=8.1, 0.9 Hz, 1H), 7.73 (dd, J=8.1, 1.0 Hz, 1H), 7.62 (t, J=1.9 Hz, 1H), 7.53 (d, J=1.8 Hz, 2H), 7.40 (t, J=8.1 Hz, 1H), 4.30-4.20 (m, 2H), 1.86 (p, J=7.5 Hz, 2H), 1.39 (s, 18H), 1.33-1.18 (m, 6H), 0.89-0.80 (m, 3H).

¹³C NMR (101 MHz, CDCl₃) δ 158.74, 151.38, 139.19, 138.33, 136.94, 128.77, 124.56, 123.90, 121.42, 119.23, 116.15, 45.26, 35.04, 31.41, 31.22, 29.95, 26.37, 22.40, 13.92.

### Example 3—Synthesis of 2-(3,5-Di-tert-butylphenyl)-1-hexyl-1H-benzo[d]imidazol-4-amine

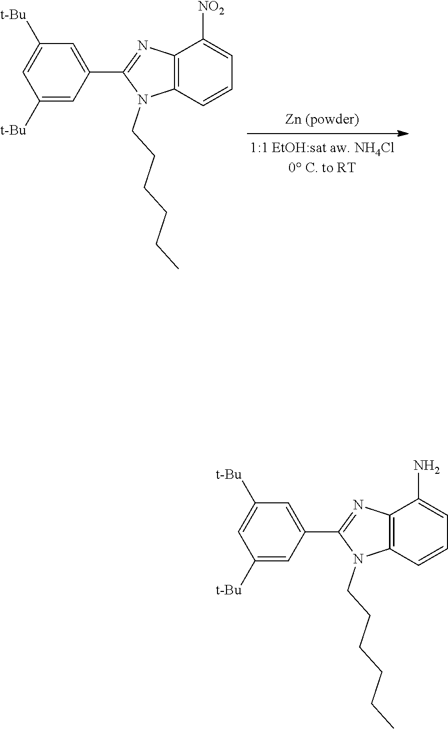 [SMILES: CCCCCCn1c(-c2cc(C(C)(C)C)cc(C(C)(C)C)c2)nc2c([N+](=O)[O-])cccc21 ; "Zn (powder)" ; CCCCCCn1c(-c2cc(C(C)(C)C)cc(C(C)(C)C)c2)nc2c(N)cccc21]

A 100-mL round-bottom flask was charged with 2-(3,5-di-tert-butylphenyl)-1-hexyl-4-nitro-1H-benzo[d]imidazole (2.10 g, 4.82 mmol), ethanol (30 mL), and sat. aq. NH₄Cl (10 mL). The mixture was stirred at room temperature under nitrogen, then Zn powder (1.58 g, 24.10 mmol)) was added in portions. The reaction was monitored by LC-MS. After stirring for 2 h EtOAc was added and the mixture was filtered through Celite. The organic layer was collected and purified by column chromatography (70:30 Hex:EtOAc). Yield: 1.96 g, 92%.

¹H NMR (400 MHz, CDCl₃) δ 7.62 (t, J=1.9 Hz, 1H), 7.56 (d, J=2.0 Hz, 2H), 7.18 (t, J=7.9 Hz, 1H), 6.87 (d, J=8.1 Hz, 1H), 6.67 (d, J=7.7 Hz, 1H), 5.00 (s, 2H), 4.18 (t, J=7.8 Hz, 2H), 1.90 (p, J=7.5 Hz, 2H), 1.39 (s, 18H), 1.36-1.19 (m, 6H), 0.92-0.78 (m, 3H).

¹³C NMR (101 MHz, CDCl₃) δ 151.57, 135.42, 124.60, 123.85, 108.1, 100.47, 45.28, 35.08, 31.43, 31.27, 29.80, 26.43, 22.45, 13.95.

### Example 4—Synthesis of 1-Hexyl-2-mesityl-1H-benzo[d]imidazol-4-amine

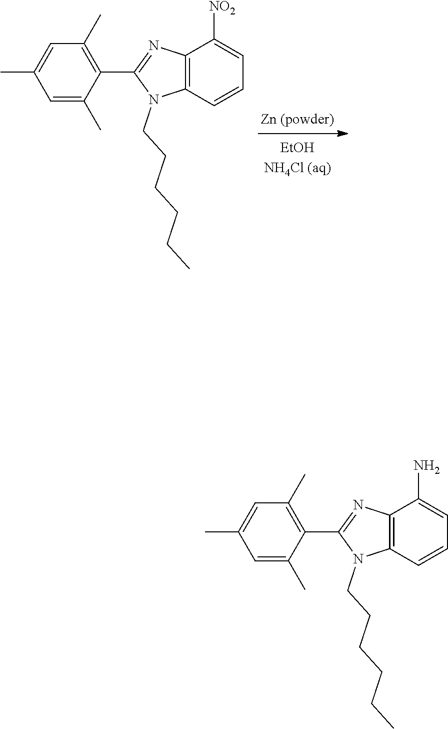 [SMILES: CCCCCCn1c(-c2c(C)cc(C)cc2C)nc2c([N+](=O)[O-])cccc21 ; "Zn (powder)" ; CCCCCCn1c(-c2c(C)cc(C)cc2C)nc2c(N)cccc21]

A 100-mL round-bottom was charged with 1-hexyl-2-mesityl-4-nitro-1H-benzo[d]imidazole (1.10 g, 3.01 mmol), ethanol (30 mL), and sat. aq. NH₄Cl (10 mL). The mixture was stirred at room temperature under nitrogen, then Zn powder (1.58 g, 24.10 mmol)) was added in portions. The reaction was monitored by LC-MS. After stirring for 2 h EtOAc was added and the mixture was filtered through Celite. The organic layer was collected and purified by column chromatography (100% EtOAc). Yield: 0.97 g, 96%.

¹H NMR (400 MHz, CDCl₃) δ 7.10 (t, J=7.9 Hz, 1H), 6.81 (dd, J=8.1, 0.9 Hz, 1H), 6.97 (s, 2H), 6.56 (dd, J=7.7, 0.9 Hz, 1H), 4.43 (s, 2H), 3.87-3.74 (m, 2H), 2.07 (s, 6H), 2.05 (s, 3H), 1.72-1.57 (m, 2H), 1.26-1.13 (m, 6H), 0.90-0.77 (m, 3H).

¹³C NMR (101 MHz, CDCl₃) δ 150.57, 139.19, 138.82, 138.07, 135.33, 132.50, 128.32, 127.31, 123.21, 105.68, 99.88, 44.15, 31.22, 29.41, 26.46, 22.38, 21.25, 19.86, 13.93.

### Example 5—Synthesis of 1-Hexyl-2-mesityl-N-(2,4,6-triisopropylphenyl)-1H-benzo[d]imidazol-4-amine

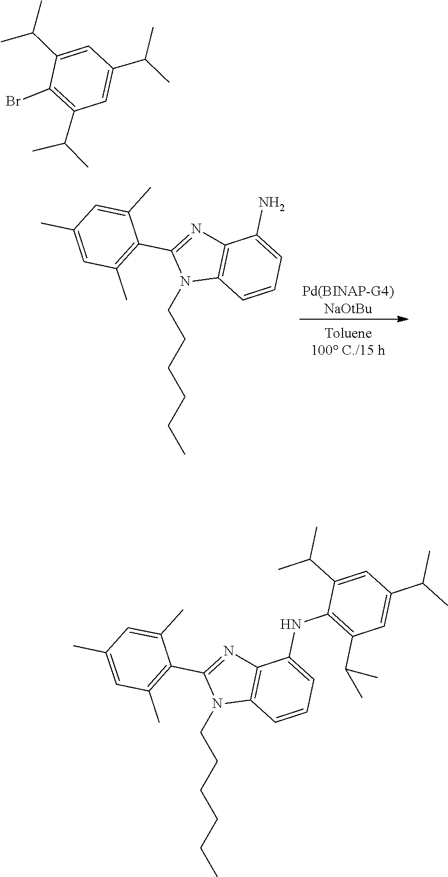

Inside a glove box, a 20-mL vial was charged with 2,4,6-triisopropylphenylbromide (0.093 g, 0.33 mmol), 1-hexyl-2-mesityl-1H-benzo[d]imidazol-4-amine (0.100 g, 0.33 mmol), Pd(BINAP)-G4 (0.030 g, 0.03 mmol), NaO^{t}Bu (0.072 g, 0.75 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Some impurities remained by ¹H NMR. The sample was purified via super-critical CO₂ column purification to give the pure product in low yield. Yield: 0.008 g, 5%).

¹H NMR (400 MHz, CDCl₃) δ 7.10 (s, 2H), 7.01 (m, 3H), 6.75 (d, J=8.0 Hz, 1H), 6.42 (s, 1H), 5.99 (d, J=7.8 Hz, 1H), 3.91-3.78 (m, 2H), 3.42-3.23 (m, J=6.8 Hz, 2H), 2.95 (h, J=6.9 Hz, 1H), 2.38 (s, 3H), 2.14 (s, 6H), 1.72 (p, J=7.7 Hz, 2H), 1.33 (d, J=6.9 Hz, 6H), 1.32-1.20 (m, 6H), 1.17 (d, J=6.9 Hz, 12H), 0.85 (t, J=6.6 Hz, 3H).

¹³C NMR (101 MHz, CDCl₃) δ 150.08, 147.56, 147.23, 140.66, 139.28, 138.21, 134.82, 132.87, 131.27, 128.37, 127.28, 123.37, 121.56, 102.18, 98.45, 44.27, 34.22, 31.21, 29.47, 28.28, 26.53, 24.16, 22.41, 21.30, 19.96, 13.95.

### Example 6—Synthesis of 3-Bromo-N-butyl-2-nitroaniline

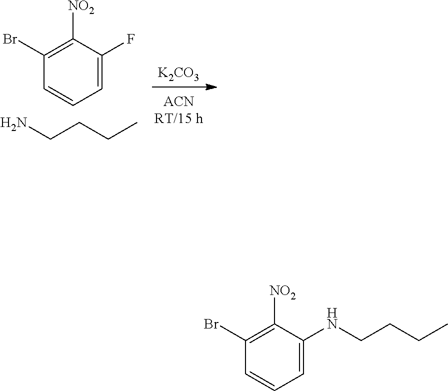

A 250-mL round-bottom was charged with 1-bromo-3-fluoro-2-nitrobenzene (10.00 g, 45.45 mmol), K₂CO₃ (7.54 g, 54.55 mmol), and acetonitrile (100 mL). n-BuNH2 (4.5 mL, 45.45 mmol) was added and the reaction was stirred for 2 d at room temperature. All volatiles were removed and the crude product was taken up in EtOAc and water. The organic layer was collected and dried over Na₂SO₄. Solids were filtered off and all volatiles were removed to yield the product as an orange solid/oil. The NMR indicates a 75:25 ratio of product to starting material. The material was used on the next step without further purification. Yield: 12.20 g, 98%.

¹H NMR (400 MHz, CDCl₃) δ 7.15 (dd, J=8.5, 7.8 Hz, 1H), 6.94 (dd, J=7.8, 1.1 Hz, 1H), 6.76 (dd, J=8.6, 1.1 Hz, 1H), 5.73 (s, 1H), 3.20 (td, J=7.1, 5.1 Hz, 2H), 1.66 (tt, J=8.6, 6.8 Hz, 2H), 1.52-1.39 (m, 2H), 0.98 (t, J=7.3 Hz, 3H).

¹³C NMR (101 MHz, CDCl₃) δ 143.83, 132.99, 121.39, 116.29, 112.38, 43.23, 31.00, 20.14, 13.76.

### Example 7—Synthesis of 3-bromo-N¹-butylbenzene-1,2-diamine

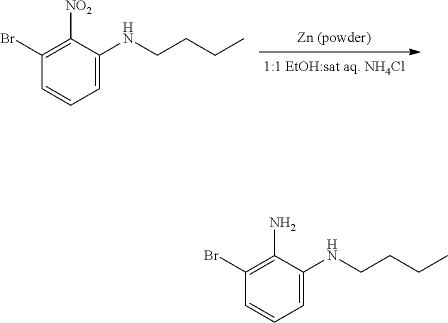

A 100 mL round bottom was charged with the 3-bromo-N-butyl-2-nitroaniline (2.64 g, 9.67 mmol), ethanol (30 mL), and sat. aq. NH₄Cl (10 mL). The mixture was stirred at room temperature under nitrogen, then Zn powder (5.06 g, 77.33 mmol)) was added in portions. The reaction was monitored by LC-MS. After stirring for 2 h EtOAc was added and the mixture was filtered through Celite. The organic layer was collected and purified by column chromatography (80:20 Hex:EtOAc). Yield: 1.72 g, 73%.

¹H NMR (400 MHz, CDCl₃) δ 6.95 (dd, J=8.1, 1.3 Hz, 1H), 6.70 (t, J=8.0 Hz, 1H), 6.65-6.58 (m, 1H), 3.76 (s, 2H), 3.35 (s, 1H), 3.12 (td, J=7.0, 3.6 Hz, 2H), 1.68 (dtd, J=8.6, 7.3, 5.9 Hz, 2H), 1.56-1.42 (m, 2H), 1.00 (t, J=7.3 Hz, 3H).

¹³C NMR (101 MHz, CDCl₃) δ 138.89, 132.35, 121.60, 120.82, 111.23, 110.41, 44.14, 31.71, 20.41, 13.95.

### Example 8—Synthesis of 4-Bromo-1-butyl-2-(3,5-di-tert-butylphenyl)-1H-benzo[d]imidazole

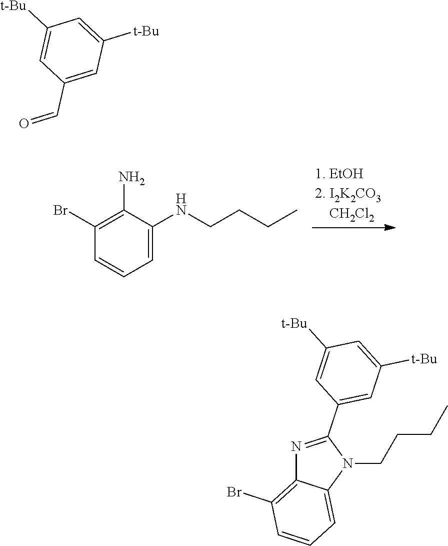

A 250-mL round-bottom flask was charged with 3-bromo-N¹-butylbenzene-1,2-diamine (1.70 g, 6.99 mmol), 3,5-di-tert-butylbenzaldehyde (1.53 g, 6.99 mmol), and EtOH (100 mL, absolute). The mixture was heated to 70° C. for 15 h. All volatiles were removed, then CH₂Cl₂ (100 mL), K₂CO₃ (2.13 g, 15.38 mmol), and 12 (1.78 g, 6.99 mmol) were added and the mixture was allowed to stir for 3 h. Water was added to the mixture and the organic layer was collected. The crude product was purified by column chromatography (80:20 Hex:EtOAc, 2nd product). Yield: 2.56 g, 83%.

¹H NMR (400 MHz, CDCl₃) δ 7.58 (t, J=1.9 Hz, 1H), 7.49 (m, 3H), 7.36 (dd, J=8.1, 0.9 Hz, 1H), 7.16 (t, J=7.9 Hz, 1H), 4.18-4.08 (m, 2H), 1.90-1.72 (m, 2H), 1.39 (s, 18H), 1.36-1.22 (m, 2H), 0.86 (t, J=7.4 Hz, 3H).

¹³C NMR (101 MHz, CDCl₃) δ 155.69, 151.08, 141.82, 135.93, 129.40, 125.21, 123.96, 123.90, 123.30, 113.31, 109.29, 44.90, 34.99, 31.95, 31.43, 19.97, 13.54.

### Example 8—Synthesis of N-(3,5-Di-tert-butylphenyl)-1-hexyl-2-mesityl-1H-benzo[d]imidazol-4-amine

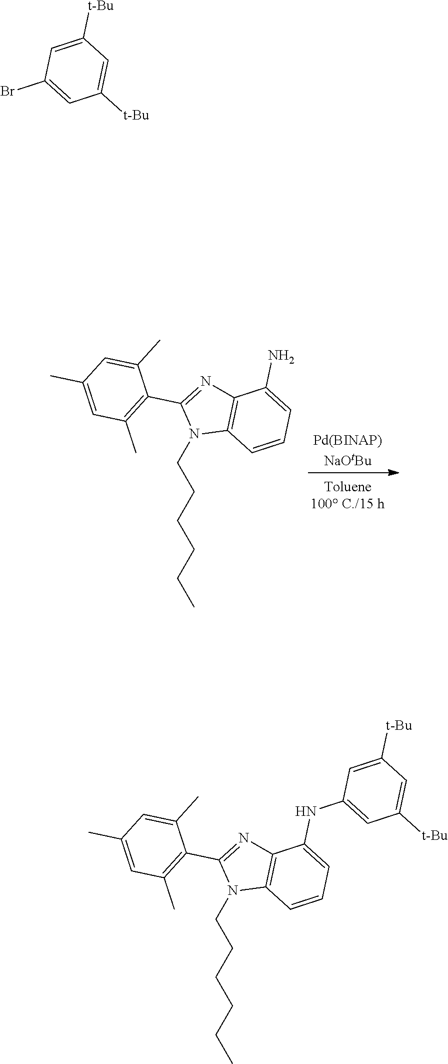 [SMILES: CC(C)(C)c1cc(Br)cc(C(C)(C)C)c1 ; CCCCCCn1c(-c2c(C)cc(C)cc2C)nc2c(N)cccc21 ; CCCCCCn1c(-c2c(C)cc(C)cc2C)nc2c(Nc3cc(C(C)(C)C)cc(C(C)(C)C)c3)cccc21 ; "Pd(BINAP)NaOtBu"]

Inside a glove box, a 20-mL vial was charged with 1-hexyl-2-mesityl-1H-benzo[d]imidazol-4-amine (0.060 g, 0.18 mmol), 1-bromo-3,5-di-tert-butylbenzene (0.053 g, 0.20 mmol), Pd(BINAP-G3) (0.009 g, 0.01 mmol), NaOtBu (0.043 g, 0.45 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Some impurities remain by ¹H NMR. The sample was purified by super-critical CO₂ column chromatography to give the clean product. Yield: 0.036 g, 38%.

¹H NMR (400 MHz, CDCl₃) δ 7.26 (d, J=1.5 Hz, 3H), 7.22 (t, J=4.5 Hz, 5H), 7.10 (q, J=1.6 Hz, 1H), 7.02 (s, 2H), 6.97-6.88 (m, 1H), 3.88 (t, J=7.7 Hz, 2H), 2.40 (s, 3H), 2.12 (s, 6H), 1.72 (h, J=6.9 Hz, 2H), 1.39 (s, 18H), 1.34-1.17 (m, 6H), 0.93-0.81 (m, 3H).

¹³C NMR (101 MHz, CDCl₃) δ 151.72, 150.62, 141.35, 139.42, 138.13, 135.99, 135.11, 132.86, 128.43, 127.05, 123.27, 115.71, 113.72, 103.63, 100.53, 44.27, 34.96, 31.52, 31.26, 29.52, 26.52, 22.42, 21.31, 19.93, 13.98.

### Example 9—Synthesis of 1-Butyl-2-(3,5-di-tert-butylphenyl)-N-(o-tolyl)-1H-benzo[d]imidazol-4-amine

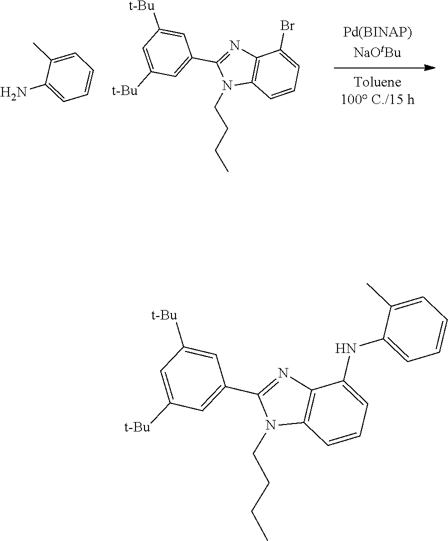

Inside a glove box, a 20-mL vial was charged with 4-bromo-1-butyl-2-(3,5-di-tert-butylphenyl)-1H-benzo[d]imidazole (0.060 g, 0.14 mmol), toluidine (0.016 g, 0.15 mmol), Pd(BINAP) (0.007 g, 0.01 mmol), NaO^{t}Bu (0.033 g, 0.34 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Yield: 0.064 g, 36%.

¹H NMR (400 MHz, CDCl₃) δ 7.60 (t, J=1.8 Hz, 1H), 7.55 (t, J=1.9 Hz, 2H), 7.31-7.26 (m, 1H), 7.23 (td, J=7.7, 1.7 Hz, 1H), 7.17 (t, J=8.0 Hz, 1H), 7.11-6.99 (m, 1H), 6.90 (ddd, J=10.6, 8.0, 0.9 Hz, 2H), 6.77-6.66 (m, 1H), 4.23-4.10 (m, 2H), 2.40 (s, 3H), 1.90 (ddt, J=9.3, 7.7, 3.7 Hz, 2H), 1.42 (s, 18H), 1.40-1.34 (m, 2H), 0.91 (t, J=7.4 Hz, 3H).

¹³C NMR (101 MHz, CDCl₃) δ 153.01, 151.18, 140.47, 136.52, 136.13, 132.95, 130.93, 130.23, 129.97, 126.55, 123.77, 123.71, 123.35, 122.65, 120.75, 104.63, 100.65, 44.76, 35.03, 32.11, 31.47, 20.09, 18.16, 13.63.

### Example 10—Synthesis of 1-Butyl-2-(3,5-di-tert-butylphenyl)-N-(2,6-diisopropylphenyl)-1H-benzo[d]imidazol-4-amine

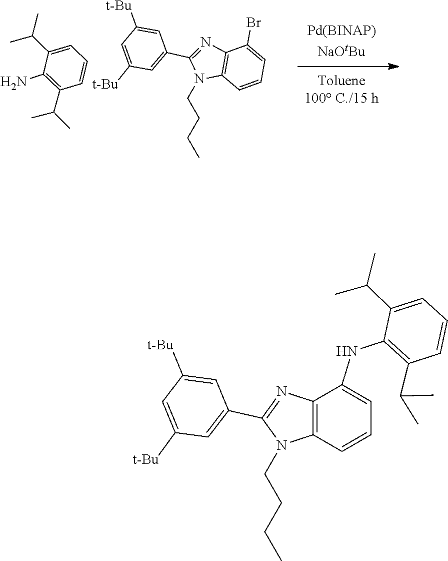

Inside a glove box, a 20-mL vial was charged with 2,6-diisopropylaniline (0.052 g, 0.29 mmol), 4-bromo-1-butyl-2-(3,5-di-tert-butylphenyl)-1H-benzo[d]imidazole (0.100 g, 0.23 mmol), Pd(BINAP) (0.011 g, 0.01 mmol), NaOtBu (0.054 g, 0.57 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Some impurities remained by ¹H NMR. The sample was purified by super critical CO₂ column chromatography to yield the pure product. Yield: 0.056 g, 46%.

¹H NMR (400 MHz, CDCl₃) δ 7.60 (dd, J=12.2, 1.9 Hz, 3H), 7.37 (dd, J=8.6, 6.5 Hz, 1H), 7.30 (d, J=8.3 Hz, 2H), 7.04 (t, J=7.9 Hz, 1H), 6.78 (d, J=8.0 Hz, 1H), 6.48 (s, 1H), 5.97 (d, J=7.8 Hz, 1H), 4.16 (dt, J=10.5, 7.5 Hz, 2H), 3.40 (hept, J=6.9 Hz, 2H), 1.94 (dq, J=9.5, 7.4 Hz, 2H), 1.44 (s, 18H), 1.42-1.35 (m, 2H), 1.20 (d, J=6.9 Hz, 12H), 0.94 (t, J=7.3 Hz, 3H).

¹³C NMR (101 MHz, CDCl₃) δ 152.43, 151.11, 148.19, 140.36, 135.81, 135.02, 131.06, 130.08, 127.27, 123.84, 123.76, 123.70, 123.63, 102.43, 98.68, 44.78, 35.04, 32.23, 31.50, 28.17, 23.58, 20.19, 13.67.

### Example 11—Synthesis of N-(2,7-Di-tert-butylanthracen-9-yl)-1-hexyl-2-mesityl-1H-benzo[d]imidazol-4-amine

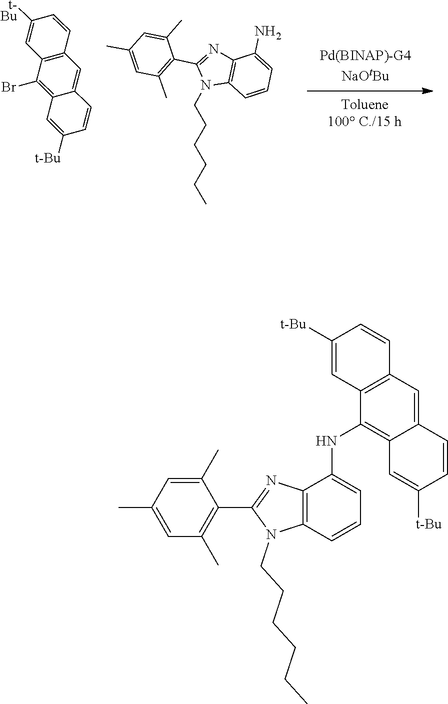

Inside a glove box, a 20-mL vial was charged with 9-bromo-2,7-di-tert-butylanthracene (0.121 g, 0.33 mmol), 1-hexyl-2-mesityl-1H-benzo[d]imidazol-4-amine (0.100 g, 0.30 mmol), Pd(BINAP) (0.030 g, 0.03 mmol), NaO^{t}Bu (0.072 g, 0.75 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Some impurities remained by ¹H NMR. The sample was purified by super critical CO₂ column chromatography to yield the pure product. Yield: 0.027 g, 15%.

¹H NMR (400 MHz, CDCl₃) δ 8.36 (s, 1H), 8.32-8.26 (m, 2H), 8.03 (d, J=8.9 Hz, 2H), 7.61 (dd, J=8.9, 1.9 Hz, 2H), 7.54 (s, 1H), 7.10 (s, 2H), 6.96 (t, J=7.9 Hz, 1H), 6.86 (dd, J=8.1, 0.9 Hz, 1H), 6.08 (dd, J=7.8, 0.9 Hz, 1H), 4.02-3.90 (m, 2H), 2.46 (s, 3H), 2.26 (s, 6H), 1.79 (h, J=9.6, 8.6 Hz, 2H), 1.39 (s, 18H), 1.36-1.24 (m, 2H), 0.91 (t, J=6.7 Hz, 3H).

¹³C NMR (101 MHz, CDCl₃) δ 150.55, 147.60, 140.27, 139.36, 138.21, 134.95, 133.25, 132.10, 130.54, 129.02, 128.43, 128.22, 127.33, 124.49, 123.72, 123.24, 118.73, 104.25, 99.54, 44.31, 35.15, 31.28, 30.99, 29.52, 26.54, 22.43, 21.34, 19.94, 13.97.

### Example 12—Synthesis of 1-Hexyl-N-(2-isopropylphenyl)-2-mesityl-1H-benzo[d]imidazol-4-amine

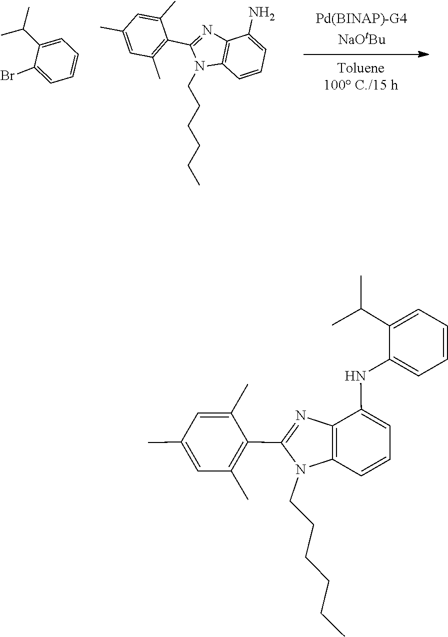

Inside a glove box, a 20-mL vial was charged with the 1-bromo-2-isopropylbenzene (0.065 g, 0.33 mmol), 1-hexyl-2-mesityl-1H-benzo[d]imidazol-4-amine (0.100 g, 0.30 mmol), Pd(BINAP) (0.030 g, 0.03 mmol), NaO^{t}Bu (0.072 g, 0.75 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 80:20). Some impurities remained by ¹H NMR. The sample was purified by super critical CO₂ column chromatography to yield the pure product. Yield: 0.112 g, 83%.

¹H NMR (400 MHz, CDCl₃) δ 7.60 (dd, J=7.8, 1.5 Hz, 1H), 7.43 (dd, J=7.6, 1.7 Hz, 1H), 7.27 (td, J=7.5, 1.9 Hz, 1H), 7.20 (td, J=7.5, 1.5 Hz, 1H), 7.17 (d, J=7.9 Hz, 1H), 7.06 (s, 2H), 6.92 (dd, J=8.0, 0.9 Hz, 1H), 6.81 (s, 1H), 6.78 (dd, J=7.9, 0.9 Hz, 1H), 3.98-3.84 (m, 2H), 3.46 (hept, J=6.8 Hz, 1H), 2.43 (s, 3H), 2.19 (s, 6H), 1.84-1.68 (m, 2H), 1.32 (d, J=6.9 Hz, 6H), 1.29 (m, 4H), 0.96-0.86 (m, 3H).

¹³C NMR (101 MHz, CDCl₃) δ 150.58, 142.73, 139.39, 138.88, 138.20, 138.12, 135.17, 132.75, 128.49, 127.30, 126.31, 126.22, 124.28, 124.08, 123.20, 103.40, 100.08, 44.29, 31.29, 29.54, 27.66, 26.55, 23.47, 22.47, 21.34, 20.03, 14.03.

### Example 13—Synthesis of 4-Bromo-1-butyl-2-(naphthalen-1-yl)-1H-benzo[d]imidazole

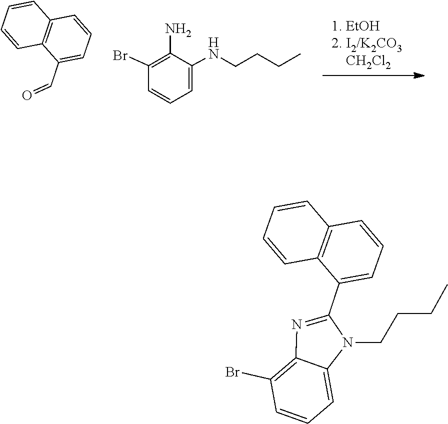

A 100-mL round-bottom flask was charged with 3-bromo-N¹-butylbenzene-1,2-diamine (1.62 g, 6.66 mmol), 1-naphthaldehyde (0.91 mL, 6.66 mmol), and EtOH (50 mL, absolute). The mixture was heated to 70° C. for 15 h. All volatiles were removed, then CH₂Cl₂ (50 mL), K₂CO₃ (2.03 g, 14.66 mmol), and 12 (1.69 g, 6.66 mmol) were added and the mixture was allowed to stir for 3 h. Water was added to the mixture and the organic layer was collected. The crude product was purified by column chromatography (60:40 Hex:EtOAc, 2nd product). Yield: 1.84 g, 73%.

¹H NMR (400 MHz, CDCl₃) δ 8.03 (dt, J=8.3, 1.2 Hz, 1H), 7.96-7.91 (m, 1H), 7.70 (dt, J=7.0, 1.2 Hz, 1H), 7.60 (dtd, J=7.1, 4.4, 3.5, 2.3 Hz, 2H), 7.57-7.50 (m, 2H), 7.47 (dq, J=8.3, 1.8, 1.4 Hz, 1H), 7.44 (dd, J=8.0, 1.0 Hz, 1H), 7.23 (tt, J=7.9, 1.5 Hz, 1H), 3.98 (t, J=7.4 Hz, 2H), 1.69-1.49 (m, 2H), 1.19-0.99 (m, 2H), 0.67 (tt, J=7.4, 1.5 Hz, 3H).

¹³C NMR (101 MHz, CDCl₃) δ 153.27, 142.13, 135.53, 133.47, 132.29, 130.46, 128.97, 128.40, 127.76, 127.20, 126.45, 125.34, 124.98, 123.58, 113.60, 109.51, 109.49, 44.76, 31.63, 19.70, 13.37.

### Example 14—Synthesis of 4-Bromo-1-butyl-2-isopropyl-1H-benzo[d]imidazole

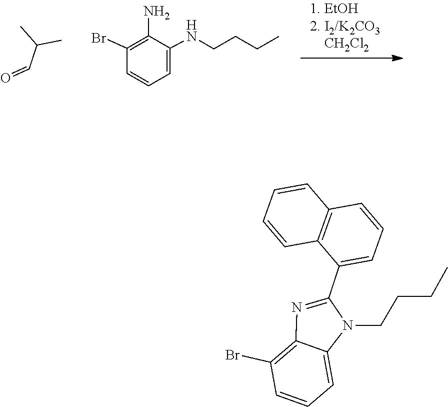

A 100-mL round-bottom flask was charged with 3-bromo-N¹-butylbenzene-1,2-diamine (1.50 g, 6.17 mmol), isobutyraldehyde (0.56 mL, 6.17 mmol), and EtOH (50 mL, absolute). The mixture was heated to 70° C. for 15 h. All volatiles were removed, then CH₂Cl₂ (50 mL), K₂CO₃ (1.88 g, 13.57 mmol), and 12 (1.57 g, 6.17 mmol) were added and the mixture was allowed to stir for 3 h. Water was added to the mixture and the organic layer was collected. The crude product was purified by column chromatography (60:40 Hex:EtOAc, 2nd product). Yield: 1.55 g, 89%.

¹H NMR (400 MHz, CDCl₃) δ 7.39 (dd, J=7.7, 0.9 Hz, 1H), 7.24 (dd, J=8.0, 1.0 Hz, 1H), 7.06 (t, J=7.9 Hz, 1H), 4.13-4.05 (m, 2H), 3.20 (hept, J=6.9 Hz, 1H), 1.76 (tt, J=9.2, 6.8 Hz, 2H), 1.47 (d, J=6.9 Hz, 6H), 1.39 (dt, J=14.8, 7.4 Hz, 4H), 0.97 (t, J=7.4 Hz, 3H).

¹³C NMR (101 MHz, CDCl₃) δ 160.44, 141.54, 135.61, 124.64, 122.64, 112.85, 108.68, 43.68, 32.15, 26.74, 21.71, 20.19, 13.76.

### Example 15—Synthesis of 4-Bromo-1-butyl-1,3-dihydro-2H-benzo[d]imidazol-2-one

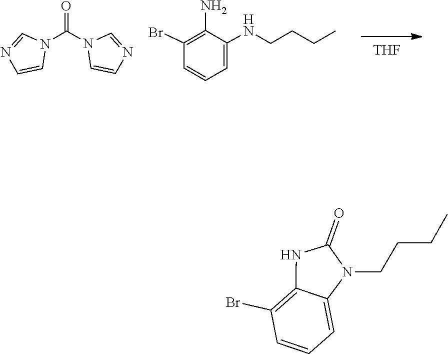

A 20-mL vial was charged with 3-bromo-N¹-butylbenzene-1,2-diamine (0.589 g, 2.42 mmol) and THF (10 mL, not anhydrous). 1,1′-Carbonyldimidazole (0.393 g, 2.42 mmol) was added and the mixture was heated to 55° C. for 15 h. All volatiles were removed and the crude product was purified by column chromatography (Hex:EtOAc 60:40) to yield the pure product. Yield: 0.493 g, 76%.

¹H NMR (400 MHz, CDCl3) δ 9.45 (s, 1H), 7.20 (dd, J=7.9, 1.2 Hz, 1H), 6.99 (t, J=7.9 Hz, 1H), 6.94 (dt, J=7.9, 1.0 Hz, 1H), 3.90 (t, J=7.2 Hz, 2H), 1.89-1.68 (m, 2H), 1.60-1.28 (m, 2H), 0.98 (t, J=7.4 Hz, 3H).

¹³C NMR (101 MHz, CDCl3) δ 154.61, 131.17, 127.63, 123.95, 122.34, 106.76, 102.29, 41.02, 30.37, 20.05, 13.72.

### Example 16—Synthesis of 4-Bromo-1-butyl-2-chloro-1H-benzo[d]imidazole

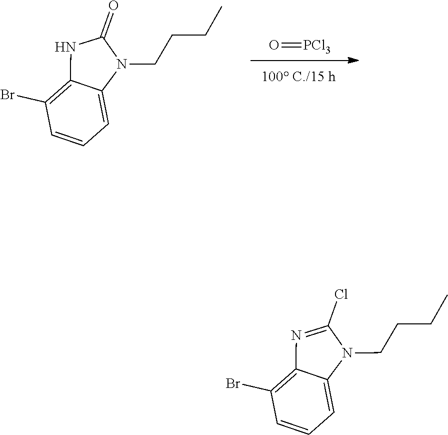

A 20-mL vial was charged with the 4-bromo-1-butyl-1,3-dihydro-2H-benzo[d]imidazol-2-one (0.493 g, 1.83 mmol) and POCl₃ (2.05 mL, 21.98 mmol). The neat mixture was heated under nitrogen at 100° C. overnight. The reaction was cooled and CH₂Cl₂ (8 mL) was added then water was added slowly (quenching was sluggish at first, but became very fast with time). The organic layer was collected and dried over Na₂SO₄. The solids were filtered off and all volatiles were removed. The crude product looked good by NMR. No further purification was needed. Yield: 0.498 g, 95%.

¹H NMR (400 MHz, CDCl₃) δ 7.34 (dd, J=7.8, 0.9 Hz, 1H), 7.18 (dd, J=8.1, 0.9 Hz, 1H), 7.06 (t, J=8.0 Hz, 1H), 4.08 (t, J=7.3 Hz, 2H), 1.69 (dq, J=9.2, 7.3 Hz, 2H), 1.37-1.19 (m, 2H), 0.86 (t, J=7.4 Hz, 3H).

¹³C NMR (101 MHz, CDCl₃) δ 141.07, 139.59, 135.04, 125.90, 124.23, 112.02, 109.06, 44.91, 31.18, 19.82, 13.55.

### Example 17—Synthesis of 9-(4-Bromo-1-butyl-1H-benzo[d]imidazol-2-yl)-3,6-di-tert-butyl-9H-carbazole

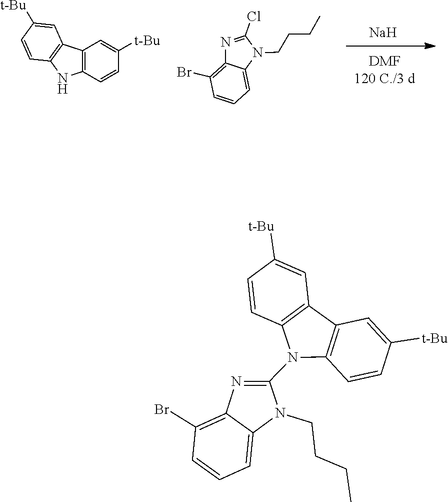

A 20-mL vial was charged with NaH (0.031 g, 1.31 mmol) in the glovebox. The vial was taken out of the glovebox and a DMF (6 mL) solution of the 3,6-di-tert-butyl-9H-carbazole (Cbz, 0.365 g, 1.31 mmol) and 4-bromo-1-butyl-2-chloro-1H-benzo[d]imidazole (0.365 g, 0.65 mmol) were added to the vial. The vial was heated to 120° C. over the weekend. Hexanes and water were added and the organic layer was collected. All volatiles were removed and the crude product was purified by column chromatography (Hex:EtOAc 90:10). The product and starting Cbz nearly co-elute. Yield: 0.064 g, 18%.

¹H NMR (400 MHz, CDCl₃) δ 8.12 (dd, J=2.0, 0.7 Hz, 2H), 7.59 (dd, J=7.8, 0.9 Hz, 1H), 7.52-7.44 (m, 3H), 7.28 (t, J=8.0 Hz, 1H), 7.26 (dd, J=8.5, J=0.6 Hz, 2H), 4.08 (t, J=7.1 Hz, 2H), 1.45 (s, 18H), 1.37-1.25 (m, 2H), 1.06-0.95 (m, 2H), 0.61 (t, J=7.4 Hz, 3H).

¹³C NMR (101 MHz, CDCl₃) δ 145.48, 144.43, 140.93, 138.96, 134.79, 125.79, 124.34, 124.22, 123.88, 116.40, 113.82, 110.25, 109.61, 44.63, 34.81, 31.95, 31.12, 19.56, 13.20.

### Example 18—Synthesis of 1-Butyl-2-(3,6-di-tert-butyl-9H-carbazol-9-yl)-N-(o-tolyl)-1H-benzo[d]imidazol-4-amine

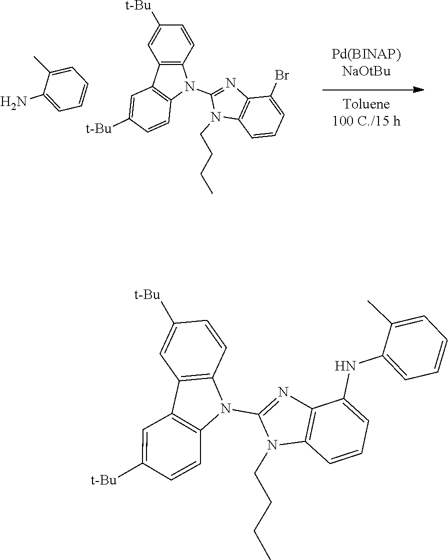

Inside a glove box, a 20-mL vial was charged with ortho-toluidine (0.014 g, 0.13 mmol), 9-(4-bromo-1-butyl-1H-benzo[d]imidazol-2-yl)-3,6-di-tert-butyl-9H-carbazole (0.064 g, 0.12 mmol), Pd(BINAP) (0.006 g, 0.01 mmol), NaO^{t}Bu (0.029 g, 0.30 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 90:10). Some impurities remain by ¹H NMR, but the product was tested in the PPR anyway. Yield: 0.050 g, 74%.

¹H NMR (400 MHz, CDCl₃) δ 8.20 (d, J=1.9 Hz, 2H), 7.63-7.57 (m, 1H), 7.54 (dd, J=8.6, 1.9 Hz, 2H), 7.32-7.25 (m, 5H), 7.07 (td, J=7.4, 1.2 Hz, 1H), 6.99 (dd, J=8.1, 0.8 Hz, 1H), 6.95 (dd, J=7.9, 0.8 Hz, 1H), 6.69 (s, 1H), 4.07 (t, J=7.2 Hz, 2H), 2.39 (s, 3H), 1.71-1.59 (m, 3H), 1.53 (s, 18H), 1.11 (dq, J=9.5, 7.4 Hz, 2H), 0.67 (t, J=7.4 Hz, 3H).

¹³C NMR (101 MHz, CDCl₃) δ 144.24, 142.63, 140.03, 139.37, 137.06, 134.96, 131.57, 131.05, 130.71, 126.63, 124.35, 124.10, 124.07, 123.15, 121.42, 116.48, 110.08, 104.48, 100.70, 44.25, 34.86, 32.03, 31.33, 19.72, 18.08, 13.30.

High Throughput Synthesis Using CM3 Liquid Handler—General Procedure for Examples 15 to 30

The brominated compounds and amines were provided for a Buchwald-Hartwig cross-coupling reaction in a high throughput sequence beginning with CM3 manipulation.

Brominated starting materials were provided and reacted with excess amine (2:1). All reactants/reagents were delivered in solution (toluene) with the exception of sodium t-butoxide and the catalyst (each weighed as solids). Reactions were diluted with additional reaction solvent to ˜10 mL before overnight reaction. The following day reaction conversion was checked via UPLC. After 16 hours at 95° C. conversion was high enough to proceed with purification.

Purification consisted of three processes: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25-mL Biotage ISOLUTE® Phase separator column. An additional 5 mL of chloroform was added and the organic phase was collected after gravity filtration. The collected material was poured into a GL Sciences 20-mL InertSep PS-SL filter and gravity filtered again. One wash of 5 mL of chloroform was similarly used to rinse the phase separation column and the InertSep filter. A final rinse of the silica pad was performed with 5 mL of ethyl acetate and the collected samples were concentrated over 10 hours at 80° C. under vacuum on a Savant SpeedVac, which ramped at 5 Torr/min. The solids were purified on the SFC.

Preparative SFC was used to purify using a 1-AA 130 Å 5 μm OBD 30×150 mm column using CO₂ as mobile phase A and 75% acetonitrile:25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL, and the injection volume was 960 μL. The desired compounds were identified by mass spectrometry.

### Example 19 —1-Butyl-2-isopropyl-N-(2-isopropylphenyl)-1H-benzo[d]imidazol-4-amine

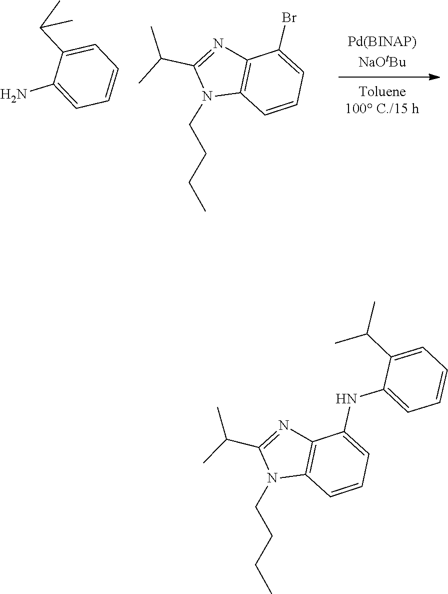

Used “CM3 Synthesis General Procedure”. Yield: 0.096 g, 54%.

¹H NMR (400 MHz, CDCl₃) δ 7.57 (dd, J=7.9, 1.4 Hz, 1H), 7.44 (dd, J=7.6, 1.7 Hz, 1H), 7.26 (td, J=7.6, 1.8 Hz, 1H), 7.19 (td, J=7.4, 1.5 Hz, 1H), 7.12 (t, J=7.9 Hz, 1H), 6.83 (dd, J=8.0, 0.9 Hz, 1H), 6.79 (s, 2H), 6.76 (dd, J=7.9, 0.9 Hz, 2H), 4.22-4.08 (m, 2H), 3.46 (hept, J=6.8 Hz, 1H), 3.28 (hept, J=6.9 Hz, 1H), 1.89 (tt, J=9.1, 6.8 Hz, 2H), 1.55 (d, J=6.8 Hz, 6H), 1.55-1.45 (m, 4H), 1.37 (d, J=6.8 Hz, 6H), 1.07 (t, J=7.4 Hz, 3H).

¹³C NMR (101 MHz, CDCl₃) δ 157.48, 142.02, 139.19, 137.40, 135.50, 132.21, 126.33, 126.12, 123.81, 123.22, 122.73, 103.73, 99.81, 43.53, 32.34, 27.75, 26.59, 23.37, 22.01, 20.35, 13.89.

### Example 20—Synthesis of 1-Butyl-2-isopropyl-N-(trimethylsilylmethyl)-1H-benzo[d]imidazol-4-amine

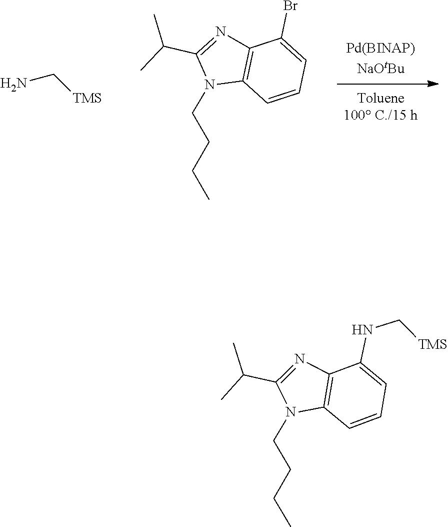 [SMILES: C[Si](C)(C)CN ; CCCCn1c(C(C)C)nc2c(Br)cccc21 ; CCCCn1c(C(C)C)nc2c(NC[Si](C)(C)C)cccc21]

Used “CM3 Synthesis General Procedure”. Yield: 0.061 g, 34%.

¹H NMR (400 MHz, CDCl₃) δ 7.15 (t, J=7.9 Hz, 1H), 6.66 (dd, J=8.1, 0.9 Hz, 1H), 6.48 (dd, J=7.8, 0.8 Hz, 1H), 4.88 (s, 1H), 4.14-4.02 (m, 2H), 3.29-3.08 (m, 1H), 2.72 (s, 2H), 1.90-1.72 (m, 2H), 1.47 (d, J=6.8 Hz, 6H), 1.46-1.38 (m, 4H), 1.01 (t, J=7.4 Hz, 3H), 0.25 (s, 9H).

¹³C NMR (101 MHz, CDCl₃) δ 156.52, 142.47, 134.77, 123.32, 100.57, 97.70, 43.41, 33.25, 32.26, 26.48, 21.92, 20.26, 13.82, −2.37.

### Example 21—Synthesis of 1-Butyl-2-(naphthalen-1-yl)-N-(o-tolyl)-1H-benzo[d]imidazol-4-amine

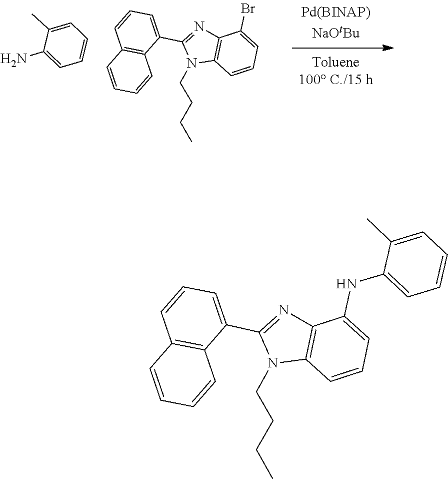

Used “CM3 Synthesis General Procedure”. Yield: 0.110 g, 51%.

¹H NMR (400 MHz, CDCl₃) δ 8.05 (dt, J=8.2, 1.1 Hz, 1H), 8.00-7.94 (m, 1H), 7.76-7.70 (m, 2H), 7.64 (dd, J=8.2, 7.0 Hz, 1H), 7.57 (ddd, J=8.1, 6.7, 1.4 Hz, 2H), 7.51 (ddd, J=8.2, 6.8, 1.4 Hz, 1H), 7.28-7.24 (m, 1H), 7.24-7.17 (m, 2H), 7.04 (td, J=7.4, 1.3 Hz, 1H), 6.95 (dd, J=8.1, 0.9 Hz, 1H), 6.89 (dd, J=7.9, 0.9 Hz, 1H), 6.73 (s, 1H), 3.99 (t, J=7.4 Hz, 2H), 2.37 (s, 3H), 1.66 (tt, J=9.0, 6.8 Hz, 2H), 1.13 (hept, J=7.7 Hz, 2H), 0.71 (t, J=7.4 Hz, 3H).

¹³C NMR (101 MHz, CDCl₃) δ 150.41, 140.23, 136.89, 135.61, 133.64, 132.99, 132.52, 130.98, 130.69, 130.23, 128.82, 128.38, 127.13, 126.55, 126.45, 125.54, 125.08, 123.64, 122.97, 121.45, 104.31, 100.55, 44.51, 31.73, 19.78, 18.08, 13.41.

### Example 22—Synthesis of 1-Butyl-N-(3,5-di-tert-butylphenyl)-2-isopropyl-1H-benzo[d]imidazol-4-amine

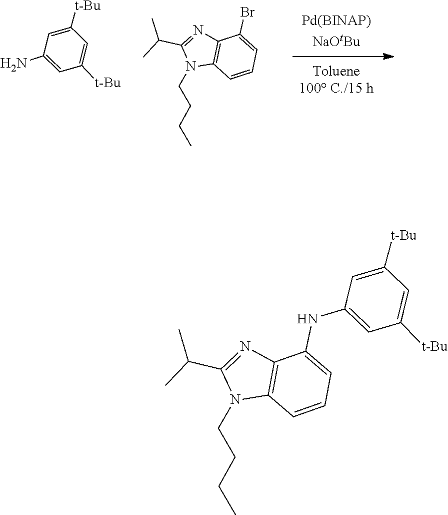 [SMILES: CC(C)(C)c1cc(N)cc(C(C)(C)C)c1 ; CCCCn1c(C(C)C)nc2c(Br)cccc21 ; CCCCn1c(C(C)C)nc2c(Nc3cc(C(C)(C)C)cc(C(C)(C)C)c3)cccc21]

Used “CM3 Synthesis General Procedure”. Yield: 0.112 g, 62%.

¹H NMR (400 MHz, CDCl₃) δ 7.31 (d, J=1.7 Hz, 2H), 7.22-7.18 (m, 2H), 7.16 (q, J=2.3, 1.7 Hz, 1H), 7.13 (s, 1H), 6.88 (h, J=4.0 Hz, 1H), 4.23-4.10 (m, 2H), 3.28 (hept, J=6.9 Hz, 1H), 1.89 (tt, J=9.1, 6.8 Hz, 2H), 1.55 (d, J=6.9 Hz, 6H), 1.50 (dd, J=8.6, 6.5 Hz, 4H), 1.45 (s, 18H), 1.07 (t, J=7.4 Hz, 3H).

¹³C NMR (101 MHz, CDCl₃) δ 157.54, 151.64, 141.55, 135.82, 135.56, 132.40, 122.78, 115.71, 114.21, 103.80, 100.14, 43.51, 35.00, 32.35, 31.61, 26.55, 22.06, 20.33, 13.89.

### Example 23—Synthesis of N-(Adamantan-1-yl)-1-butyl-2-isopropyl-1H-benzo[d]imidazol-4-amine

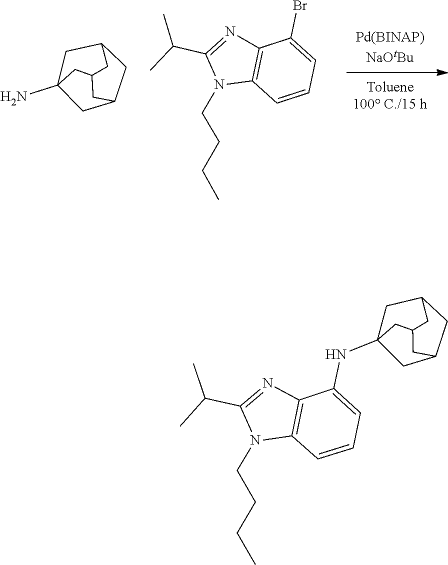

Used “CM3 Synthesis General Procedure”. Yield: 0.012 g, 7%.

¹H NMR (400 MHz, CDCl₃) δ 7.11-6.94 (m, 1H), 6.69 (d, J=7.9 Hz, 1H), 6.61 (d, J=7.9 Hz, 1H), 4.93 (br s, 1H), 4.10-4.00 (m, 2H), 3.16 (hept, J=6.7 Hz, 1H), 2.21-2.11 (m, 9H), 1.84-1.71 (m, 8H), 1.44 (d, J=6.9 Hz, 6H), 1.42-1.36 (m, 2H), 0.98 (t, J=7.3 Hz, 3H). No ¹³C NMR data due to small quantity of sample.

### Example 24—Synthesis of 1-Butyl-N-cyclohexyl-2-isopropyl-1H-benzo[d]imidazol-4-amine

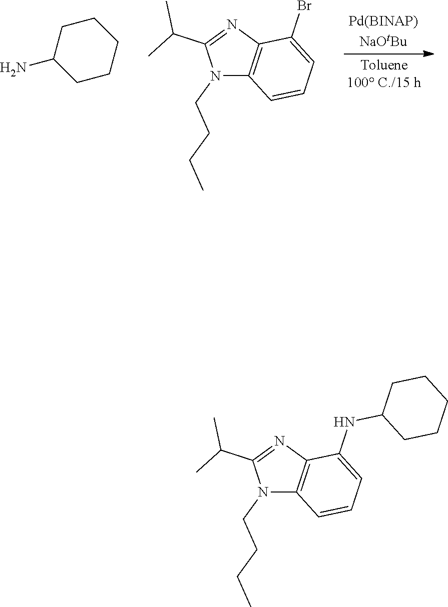

1 Used “CM3 Synthesis General Procedure”. Yield: 0.085 g, 47%.

¹H NMR (400 MHz, CDCl₃) δ 7.12 (t, J=7.9 Hz, 1H), 6.65 (dd, J=8.0, 0.9 Hz, 1H), 6.48-6.36 (m, 1H), 4.89 (s, 1H), 4.15-4.02 (m, 2H), 3.48 (t, J=9.2 Hz, 1H), 3.21 (hept, J=6.9 Hz, 1H), 2.29-2.16 (m, 2H), 1.94-1.68 (m, 5H), 1.48 (d, J=6.9 Hz, 6H), 1.47-1.28 (m, 6H), 1.02 (t, J=7.4 Hz, 3H).

¹³C NMR (101 MHz, CDCl₃) δ 156.57, 139.40, 135.18, 131.04, 123.15, 100.72, 97.44, 51.62, 43.38, 33.53, 32.28, 26.50, 26.12, 25.39, 21.98, 20.29, 13.84.

### Example 25—Synthesis of 1-Butyl-2-isopropyl-N-(2,3,5,6-tetrafluorophenyl)-1H-benzo[d]imidazol-4-amine

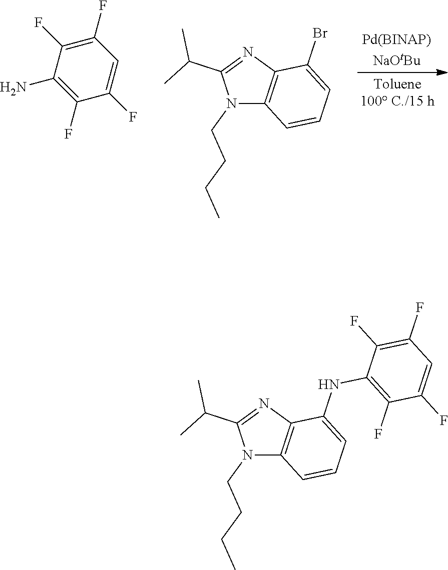

Used “CM3 Synthesis General Procedure”. Yield: 0.056 g, 31%.

¹H NMR (400 MHz, CDCl₃) δ 7.13 (t, J=7.9 Hz, 1H), 6.95 (dd, J=8.1, 0.8 Hz, 1H), 6.87-6.75 (m, 2H), 6.55 (dt, J=7.0, 3.2 Hz, 1H), 4.20-4.06 (m, 2H), 3.31-3.17 (m, J=6.8 Hz, 1H), 1.83 (tt, J=9.2, 6.8 Hz, 2H), 1.49 (d, J=6.9 Hz, 6H), 1.48-1.39 (m, 2H), 1.02 (t, J=7.4 Hz, 3H).

¹³C NMR (101 MHz, CDCl₃) δ 158.46, 145.17-147.83 (m), 139.46-142.06 (m), 135.45, 133.13, 132.90, 122.27, 105.53, 102.60, 98.79 (t, J=23.2 Hz), 43.55, 32.25, 26.53, 21.90, 20.25, 13.76.

### Example 26—Synthesis of 1-Butyl-2-(naphthalen-1-yl)-N-neopentyl-1H-benzo[d]imidazol-4-amine

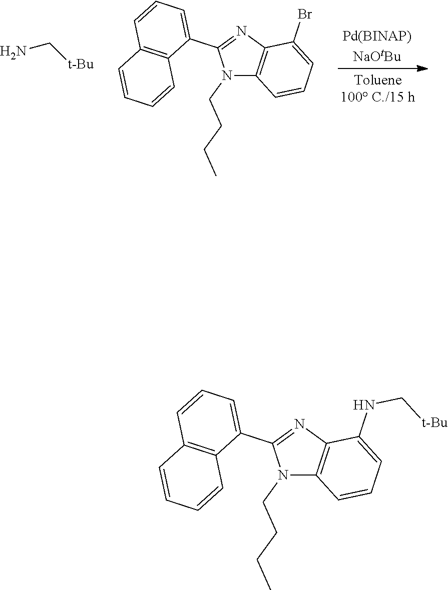 [SMILES: CC(C)(C)CN ; CCCCn1c(-c2cccc3ccccc23)nc2c(Br)cccc21 ; CCCCn1c(-c2cccc3ccccc23)nc2c(NCC(C)(C)C)cccc21]

Used “CM3 Synthesis General Procedure”. Yield: 0.091 g, 45%.

¹H NMR (400 MHz, CDCl₃) δ 8.05 (dt, J=8.2, 1.1 Hz, 1H), 8.01-7.94 (m, 1H), 7.76-7.69 (m, 2H), 7.64 (dd, J=8.2, 7.0 Hz, 1H), 7.57 (ddd, J=8.2, 6.8, 1.4 Hz, 1H), 7.50 (ddd, J=8.3, 6.8, 1.4 Hz, 1H), 7.27 (t, J=8.0 Hz, 1H), 6.82 (dd, J=8.1, 0.9 Hz, 1H), 6.56 (dd, J=7.9, 0.8 Hz, 1H), 5.19 (t, J=6.1 Hz, 1H), 3.97 (t, J=7.4 Hz, 2H), 3.19 (d, J=5.0 Hz, 2H), 1.79-1.53 (m, 2H), 1.19-1.12 (m, 2H), 1.11 (s, 9H), 0.71 (t, J=7.4 Hz, 3H).

¹³C NMR (101 MHz, CDCl₃) δ 149.54, 141.80, 135.15, 133.67, 132.68, 131.89, 130.10, 128.86, 128.67, 128.36, 127.06, 126.41, 125.65, 125.12, 124.22, 100.78, 98.06, 55.69, 44.39, 32.46, 31.76, 27.84, 19.79, 13.45.

### Example 27—Synthesis of 1-Butyl-2-(naphthalen-1-yl)-N-(trimethylsilylmethyl)-1H-benzo[d]imidazol-4-amine

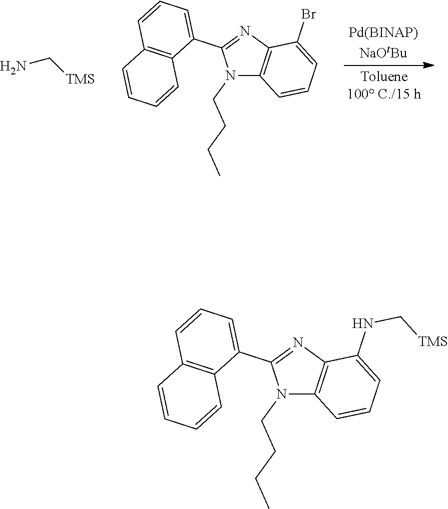 [SMILES: C[Si](C)(C)CN ; CCCCn1c(-c2cccc3ccccc23)nc2c(Br)cccc21 ; CCCCn1c(-c2cccc3ccccc23)nc2c(NC[Si](C)(C)C)cccc21]

Used “CM3 Synthesis General Procedure”. Yield: 0.074 g, 37%.

¹H NMR (400 MHz, CDCl₃) δ 8.04 (dt, J=8.2, 1.1 Hz, 1H), 8.00-7.94 (m, 1H), 7.74-7.68 (m, 2H), 7.64 (dd, J=8.2, 7.0 Hz, 1H), 7.56 (ddd, J=8.2, 6.8, 1.3 Hz, 1H), 7.49 (ddd, J=8.2, 6.8, 1.4 Hz, 1H), 7.31 (t, J=8.0 Hz, 1H), 6.84 (dd, J=8.2, 0.9 Hz, 1H), 6.62 (dd, J=7.9, 0.9 Hz, 1H), 5.02 (s, 1H), 4.03-3.87 (m, 2H), 2.78 (s, 2H), 1.77-1.48 (m, 2H), 1.18-1.06 (m, 2H), 0.71 (t, J=7.3 Hz, 3H), 0.23 (s, 9H).

¹³C NMR (101 MHz, CDCl₃) δ 149.49, 143.01, 135.03, 133.67, 132.67, 131.87, 130.10, 128.89, 128.66, 128.35, 127.03, 126.40, 125.65, 125.12, 124.28, 100.84, 98.27, 44.38, 33.43, 31.74, 19.77, 13.45, −2.37.

### Example 28—Synthesis of 1-Butyl-N-(2-isopropylphenyl)-2-(naphthalen-1-yl)-1H-benzo[d]imidazol-4-amine

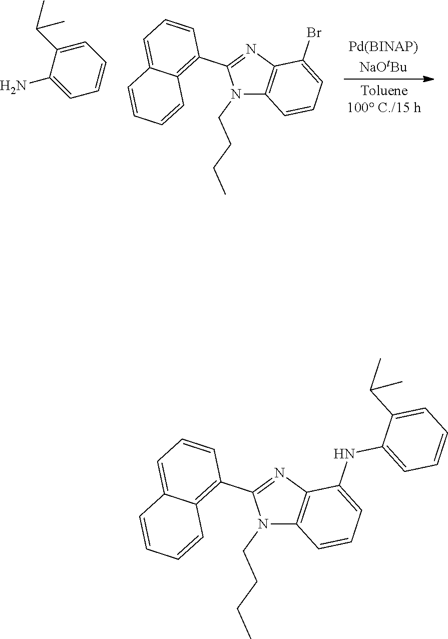

Used “CM3 Synthesis General Procedure”. Yield: 0.084 g, 42%.

¹H NMR (400 MHz, CDCl₃) δ 8.08 (dt, J=8.2, 1.1 Hz, 1H), 8.04-7.97 (m, 1H), 7.84-7.74 (m, 2H), 7.68 (dd, J=8.3, 7.0 Hz, 1H), 7.64-7.51 (m, 3H), 7.44 (dd, J=7.6, 1.7 Hz, 1H), 7.33-7.18 (m, 3H), 6.96 (dd, J=8.1, 0.8 Hz, 1H), 6.83 (s, 1H), 6.79 (dd, J=7.9, 0.9 Hz, 1H), 4.03 (t, J=7.4 Hz, 2H), 3.47 (hept, J=6.8 Hz, 1H), 1.78-1.62 (m, 2H), 1.31 (d, J=6.9 Hz, 6H), 1.17 (dd, J=6.9, 4.5 Hz, 2H), 0.75 (t, J=7.4 Hz, 3H).

¹³C NMR (101 MHz, CDCl₃) δ 150.32, 143.10, 138.71, 138.40, 135.60, 133.69, 132.76, 132.59, 130.25, 128.88, 128.50, 128.45, 127.17, 126.50, 126.39, 126.27, 125.63, 125.14, 124.54, 124.41, 123.80, 103.64, 100.07, 44.55, 31.79, 27.68, 23.51, 19.84, 13.48.

### Example 29—Synthesis of N-(Adamantan-1-yl)-1-butyl-2-(naphthalen-1-yl)-1H-benzo[d]imidazol-4-amine

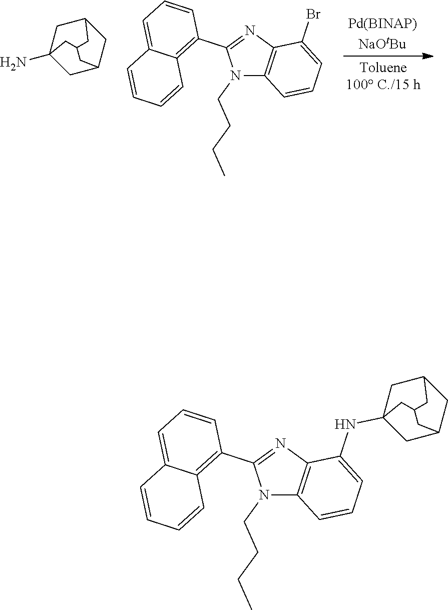

Used “CM3 Synthesis General Procedure”. Yield: 0.063 g, 31%.

¹H NMR (400 MHz, CDCl₃) δ 8.03 (dt, J=8.2, 1.1 Hz, 1H), 7.98-7.93 (m, 1H), 7.72-7.66 (m, 2H), 7.62 (dd, J=8.2, 7.0 Hz, 1H), 7.55 (ddd, J=8.2, 6.8, 1.3 Hz, 1H), 7.48 (ddd, J=8.3, 6.8, 1.4 Hz, 1H), 7.19 (t, J=8.0 Hz, 1H), 6.84 (dd, J=8.0, 0.8 Hz, 1H), 6.79 (dd, J=8.1, 0.8 Hz, 1H), 5.10 (s, 1H), 3.93 (t, J=7.4 Hz, 2H), 2.19 (s, 9H), 1.77 (t, J=2.6 Hz, 6H), 1.68-1.55 (m, 2H), 1.11 (h, J=7.4 Hz, 2H), 0.69 (t, J=7.4 Hz, 3H).

¹³C NMR (101 MHz, CDCl₃) δ 149.37, 138.94, 135.11, 133.64, 133.14, 132.66, 130.06, 128.86, 128.62, 128.30, 127.00, 126.38, 125.67, 125.08, 123.60, 104.93, 98.23, 51.80, 44.34, 42.78, 36.68, 31.72, 29.83, 19.77, 13.42.

### Example 30—Synthesis of 1-Butyl-N-cyclohexyl-2-(naphthalen-1-yl)-1H-benzo[d]imidazol-4-amine

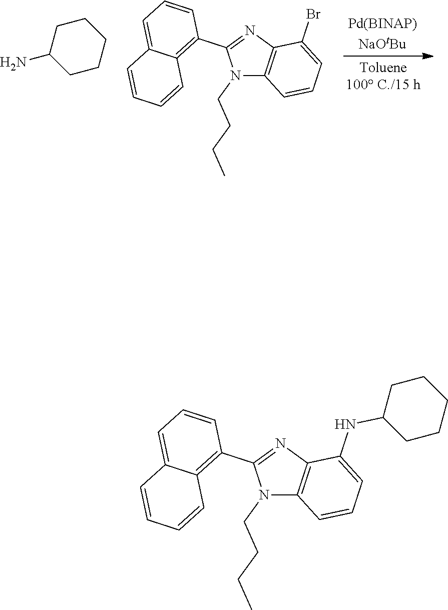

Used “CM3 Synthesis General Procedure”. Yield: 0.054 g, 27%.

¹H NMR (400 MHz, CDCl3) δ 8.03 (dt, J=8.2, 1.1 Hz, 1H), 7.99-7.94 (m, 1H), 7.73-7.67 (m, 2H), 7.62 (dd, J=8.2, 7.0 Hz, 1H), 7.55 (ddd, J=8.2, 6.8, 1.4 Hz, 1H), 7.48 (ddd, J=8.3, 6.8, 1.4 Hz, 1H), 7.24 (t, J=8.0 Hz, 1H), 6.79 (dd, J=8.1, 0.8 Hz, 1H), 6.56-6.47 (m, 1H), 5.03 (s, 1H), 3.97 (q, J=7.8, 7.4 Hz, 2H), 3.61-3.42 (m, 1H), 2.24 (dd, J=12.5, 4.0 Hz, 2H), 1.85 (dp, J=10.9, 3.6 Hz, 2H), 1.76-1.56 (m, 3H), 1.54-1.22 (m, 5H), 1.16-1.05 (m, 2H), 0.70 (t, J=7.3 Hz, 3H).

¹³C NMR (101 MHz, CDCl3) δ 149.53, 139.85, 135.28, 133.63, 132.64, 131.88, 130.06, 128.82, 128.63, 128.33, 127.05, 126.38, 125.63, 125.07, 124.20, 101.04, 97.92, 51.64, 44.38, 33.50, 31.74, 26.03, 25.32, 19.77, 13.43.

### Example 31—Synthesis of 1-Butyl-2-(naphthalen-1-yl)-N-(o-tolyl)-1H-benzo[d]imidazol-4-amine

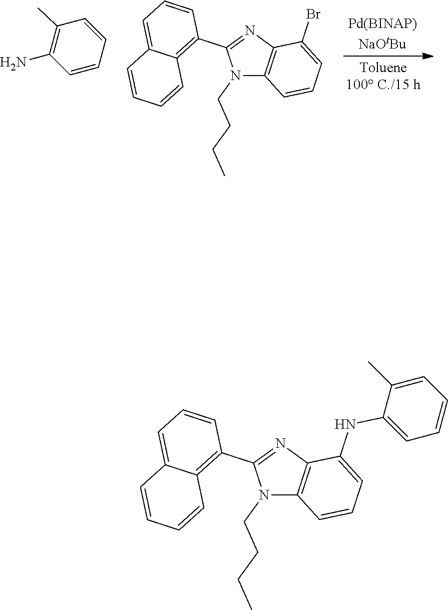

Used “CM3 Synthesis General Procedure”. Yield: 0.070 g, 35%.

¹H NMR (400 MHz, CDCl₃) δ 8.08 (dt, J=8.3, 1.1 Hz, 1H), 8.03-7.97 (m, 1H), 7.78 (td, J=7.2, 1.3 Hz, 2H), 7.70-7.51 (m, 4H), 7.34-7.23 (m, 3H), 7.08 (td, J=7.4, 1.3 Hz, 1H), 6.99 (ddd, J=11.0, 8.0, 0.9 Hz, 2H), 6.83 (s, 1H), 4.04 (t, J=7.4 Hz, 2H), 2.43 (s, 3H), 1.76-1.63 (m, 2H), 1.24-1.10 (m, 2H), 0.75 (t, J=7.4 Hz, 3H).

¹³C NMR (101 MHz, CDCl₃) δ 150.46, 140.32, 136.92, 135.69, 133.69, 133.10, 132.57, 131.06, 130.65, 130.29, 128.88, 128.46, 128.44, 127.20, 126.62, 126.51, 125.59, 125.14, 123.72, 123.00, 121.39, 104.41, 100.66, 44.55, 31.78, 19.83, 18.16, 13.48.

### Example 32—Synthesis of 1-Butyl-2-(naphthalen-1-yl)-N-(2,3,5,6-tetrafluorophenyl)-1H-benzo[d]imidazol-4-amine

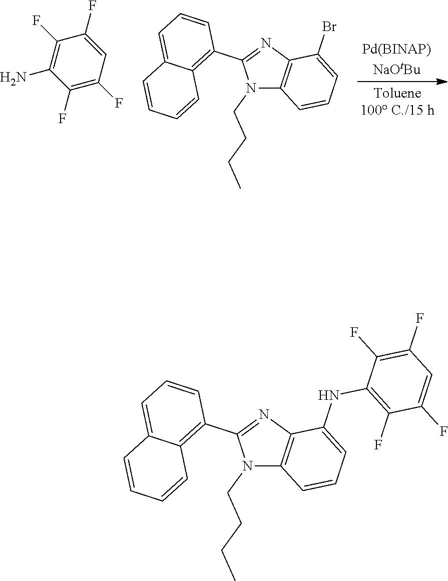

Used “CM3 Synthesis General Procedure”. Yield: 0.045 g, 22%.

¹H NMR (400 MHz, CDCl₃) δ 8.06 (dt, J=8.2, 1.1 Hz, 1H), 7.98 (dd, J=8.1, 1.4 Hz, 1H), 7.76-7.69 (m, 2H), 7.65 (dd, J=8.2, 7.0 Hz, 1H), 7.55 (dddd, J=22.7, 8.2, 6.8, 1.4 Hz, 2H), 7.31-7.25 (m, 1H), 7.11 (dd, J=8.2, 0.8 Hz, 1H), 6.94 (s, 1H), 6.84 (tt, J=9.9, 7.0 Hz, 1H), 6.65 (dt, J=7.2, 3.3 Hz, 1H), 4.02 (t, J=7.4 Hz, 2H), 1.66 (tdd, J=10.3, 8.0, 4.4 Hz, 2H), 1.12 (dd, J=14.9, 7.5 Hz, 2H), 0.71 (t, J=7.4 Hz, 3H).

¹³C NMR (101 MHz, CDCl₃) δ 151.19, 135.51, 133.84, 133.63, 133.39, 132.40, 130.39, 128.78, 128.44, 128.04, 127.27, 126.53, 125.39, 125.05, 123.35, 105.67, 103.04, 99.28 (t, J=23.1 Hz), 44.61, 31.73, 19.77, 13.39.

### Example 33—Synthesis of 1-Butyl-N-(3,5-di-tert-butylphenyl)-2-(naphthalen-1-yl)-1H-benzo[d]imidazol-4-amine

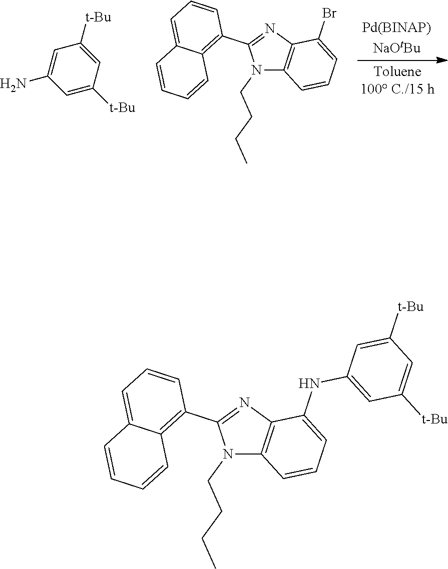 [SMILES: CC(C)(C)c1cc(N)cc(C(C)(C)C)c1 ; CCCCn1c(-c2cccc3ccccc23)nc2c(Br)cccc21 ; CCCCn1c(-c2cccc3ccccc23)nc2c(Nc3cc(C(C)(C)C)cc(C(C)(C)C)c3)cccc21]

Used “CM3 Synthesis General Procedure”. Yield: 0.114 g, 57%.

1H NMR (400 MHz, CDCl3) δ 8.08 (dt, J=8.3, 1.2 Hz, 1H), 8.02-7.97 (m, 1H), 7.80-7.72 (m, 2H), 7.67 (dd, J=8.2, 7.0 Hz, 1H), 7.56 (dddd, J=21.9, 8.3, 6.8, 1.4 Hz, 2H), 7.37-7.33 (m, 2H), 7.32 (d, J=1.8 Hz, 3H), 7.16 (t, J=1.7 Hz, 1H), 7.01 (p, J=4.3 Hz, 1H), 4.08-4.03 (m, 2H), 1.78-1.63 (m, 2H), 1.43 (s, 18H), 1.24-1.11 (m, 2H), 0.75 (t, J=7.4 Hz, 3H).

¹³C NMR (101 MHz, CDCl3) δ 151.77, 150.31, 141.37, 136.31, 135.58, 133.69, 132.96, 132.57, 130.30, 128.77, 128.45, 128.34, 127.24, 126.53, 125.53, 125.10, 123.91, 115.88, 113.99, 103.90, 100.61, 44.53, 35.00, 31.79, 31.57, 19.81, 13.48.

### Example 34—Synthesis of 1-Butyl-N,2-di(naphthalen-1-yl)-1H-benzo[d]imidazol-4-amine

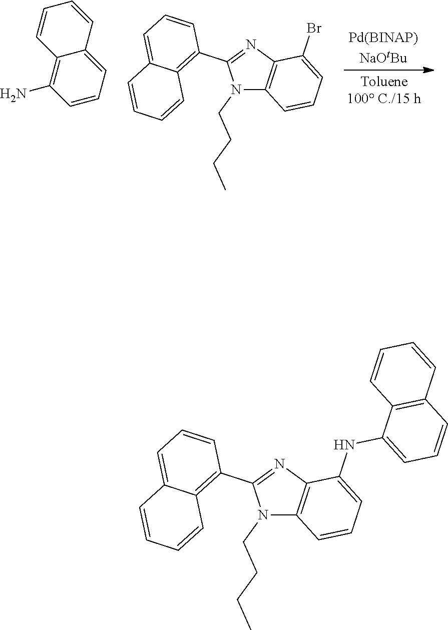

Used “CM3 Synthesis General Procedure”. Yield: 0.071 g, 31%.

¹H NMR (400 MHz, CDCl₃) δ 8.32 (dq, J=7.9, 0.8 Hz, 1H), 8.08 (dt, J=8.3, 1.1 Hz, 1H), 8.02-7.98 (m, 1H), 7.94-7.90 (m, 1H), 7.83-7.76 (m, 3H), 7.71-7.65 (m, 2H), 7.63-7.45 (m, 6H), 7.24 (t, J=8.0 Hz, 1H), 7.04-6.98 (m, 2H), 4.09-4.02 (m, 2H), 1.71 (tt, J=9.0, 6.9 Hz, 2H), 1.23-1.12 (m, 2H), 0.75 (t, J=7.4 Hz, 3H).

¹³C NMR (101 MHz, CDCl₃) δ 150.58, 137.93, 137.43, 135.69, 134.82, 133.69, 133.14, 132.56, 130.33, 128.87, 128.70, 128.47, 128.37, 128.35, 127.25, 126.54, 126.15, 125.97, 125.63, 125.55, 125.14, 123.77, 123.56, 122.81, 117.69, 104.89, 100.83, 44.58, 31.78, 19.83, 13.48.

### Example 35—Synthesis of 1-Butyl-2-isopropyl-N-(o-tolyl)-1H-benzo[d]imidazol-4-amine

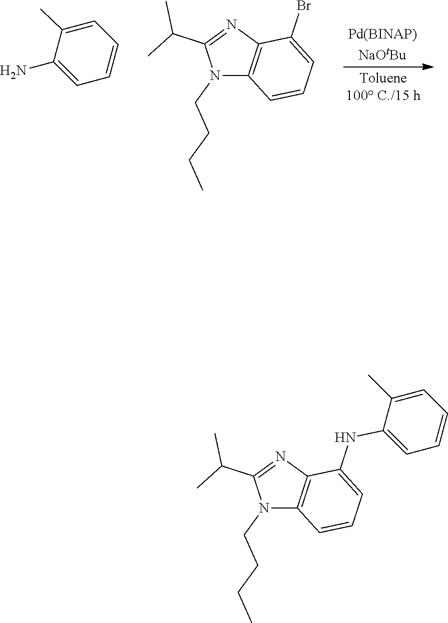

Used “CM3 Synthesis General Procedure”. Yield: 0.105 g, 58%.

¹H NMR (400 MHz, CDCl₃) δ 7.61 (dd, J=8.0, 1.3 Hz, 1H), 7.33 (dd, J=7.6, 1.6 Hz, 1H), 7.27 (td, J=7.7, 1.6 Hz, 1H), 7.16 (t, J=7.9 Hz, 1H), 7.07 (td, J=7.4, 1.3 Hz, 1H), 6.92 (dd, J=7.9, 0.9 Hz, 1H), 6.87 (dd, J=8.1, 0.9 Hz, 1H), 6.79 (s, 1H), 4.21-4.09 (m, 2H), 3.27 (hept, J=6.9 Hz, 1H), 2.47 (s, 3H), 1.96-1.81 (m, 2H), 1.55 (d, J=6.9 Hz, 6H), 1.50 (dt, J=14.8, 7.2 Hz, 2H), 1.07 (t, J=7.4 Hz, 3H).

¹³C NMR (101 MHz, CDCl₃) δ 157.66, 140.69, 136.08, 135.60, 132.53, 130.95, 129.89, 126.60, 122.67, 122.41, 120.46, 104.41, 100.34, 43.53, 32.34, 26.59, 22.02, 20.34, 18.17, 13.90.

### Example 36—Synthesis of 1-Butyl-N-(2,6-diisopropylphenyl)-2-(naphthalen-1-yl)-1H-benzo[d]imidazol-4-amine

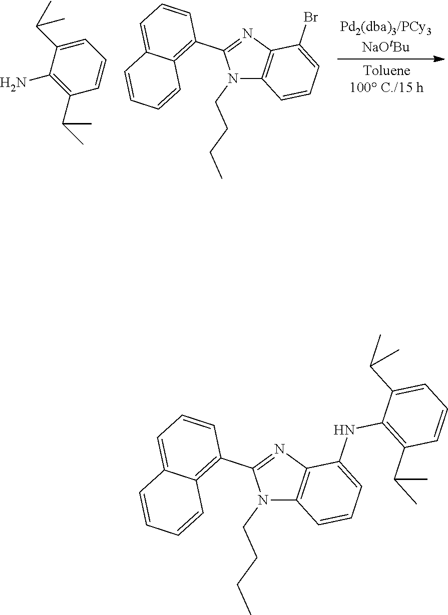

Inside a glove box, a 20-mL vial was charged with 2,6-diisopropylaniline (0.105 g, 0.59 mmol), 4-bromo-1-butyl-2-(naphthalen-1-yl)-1H-benzo[d]imidazole (0.204 g, 0.54 mmol), Pd₂dba₃ (0.025 g, 0.03 mmol), PCy₃ (0.054 mL, 0.05 mmol), NaO^{t}Bu (0.129 g, 1.34 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 90:10). Yield: 0.140 g, 55%.

¹H NMR (400 MHz, CDCl₃) δ 8.09 (dt, J=8.2, 1.1 Hz, 1H), 8.03-7.99 (m, 1H), 7.87 (dq, J=7.4, 0.9 Hz, 1H), 7.83 (dd, J=7.0, 1.3 Hz, 1H), 7.69 (dd, J=8.3, 7.0 Hz, 1H), 7.64-7.53 (m, 2H), 7.45-7.39 (m, 1H), 7.39-7.33 (m, 2H), 7.16 (t, J=7.9 Hz, 1H), 6.90 (dd, J=8.1, 0.9 Hz, 1H), 6.57 (s, 1H), 6.11 (dd, J=7.9, 0.9 Hz, 1H), 4.10-4.01 (m, 2H), 3.49 (hept, J=6.9 Hz, 2H), 1.81-1.69 (m, 2H), 1.27 (d, J=6.9 Hz, 12H), 1.24-1.16 (m, 2H), 0.77 (t, J=7.4 Hz, 3H).

¹³C NMR (101 MHz, CDCl₃) δ 149.94, 148.21, 140.57, 135.43, 135.03, 133.74, 132.67, 131.58, 130.22, 128.93, 128.63, 128.48, 127.42, 127.17, 126.49, 125.69, 125.17, 124.02, 123.85, 102.44, 98.90, 44.59, 31.88, 28.29, 24.81, 19.92, 13.50.

### Example 37—Synthesis of 1-Butyl-N-(2,6-diisopropylphenyl)-2-isopropyl-1H-benzo[d]imidazol-4-amine

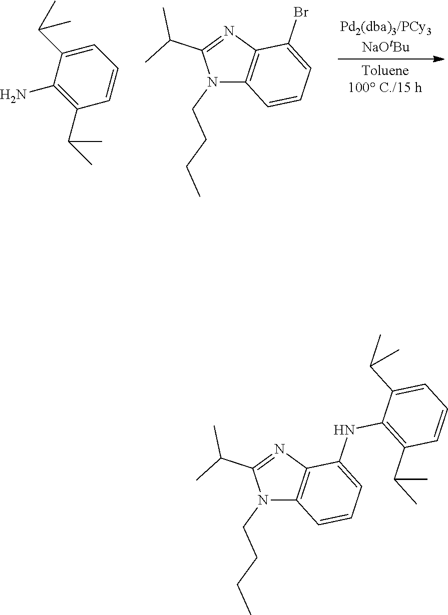

Inside a glove box, a 20-mL vial was charged with the 2,6-diisopropylaniline (0.071 g, 0.40 mmol), 1-butyl-N-(2,6-diisopropylphenyl)-2-isopropyl-1H-benzo[d]imidazol-4-amine (0.108 g, 0.37 mmol), Pd₂dba₃ (0.017 g, 0.02 mmol), PCy₃ (0.037 mL, 0.04 mmol), NaOtBu (0.088 g, 0.91 mmol), and toluene (8 mL). The vial was heated to 100° C. for 6 h and checked by LC-MS. Product was evident by LC-MS. Water and EtOAc were added and the organic layer was collected and all volatiles were removed. The crude product was purified by column chromatography (Hex:EtOAc 90:10). Yield: 0.109 g, 76%.

¹H NMR (400 MHz, CDCl₃) δ 7.40 (dd, J=8.8, 6.4 Hz, 1H), 7.34 (d, J=6.8 Hz, 2H), 7.01 (t, J=7.9 Hz, 1H), 6.74 (d, J=8.0 Hz, 1H), 6.49 (s, 1H), 5.99 (d, J=7.8 Hz, 1H), 4.22-4.10 (m, 2H), 3.42 (h, J=6.9 Hz, 2H), 3.31 (hept, J=7.0 Hz, 1H), 1.92 (tt, J=9.3, 6.8 Hz, 2H), 1.58 (d, J=6.9 Hz, 6H), 1.57-1.49 (m, 2H), 1.25 (d, J=6.9 Hz, 12H), 1.09 (t, J=7.3 Hz, 3H).

¹³C NMR (101 MHz, CDCl₃) δ 156.94, 148.01, 140.04, 135.39, 135.28, 130.78, 127.14, 123.76, 122.91, 102.32, 98.43, 43.58, 32.42, 28.17, 26.62, 24.25, 22.09, 20.42, 13.90.

General Procedure for Metal Complex Synthesis

Inside a glove box, a solution (0.5 mL, C₆D₆) of the ligand (˜15 mg, 1 equivalent for mono [2,1] complexes and 2 equivalents for bis-[2,1] metal complexes) was added over 3 min to solid M(Bn)₄ (M=Zr or Hf, ˜15 mg) at room temperature. The vial was swirled after each drop to ensure mixing. After addition, the solution was transferred to an NMR tube and checked by ¹H and ¹³C NMR. The sample was returned to the glovebox and all volatiles were removed. Ligand and MBn₄ were in contact with each other for about 0.5 hr. All volatiles were removed and the crude product was used without further purification for batch reactor testing. One or two equivalents of toluene were evident by NMR depending on the ligand:metal ratio.

### Example 38—Synthesis of Inventive Metal-Ligand Complex 1 (IMLC 1)

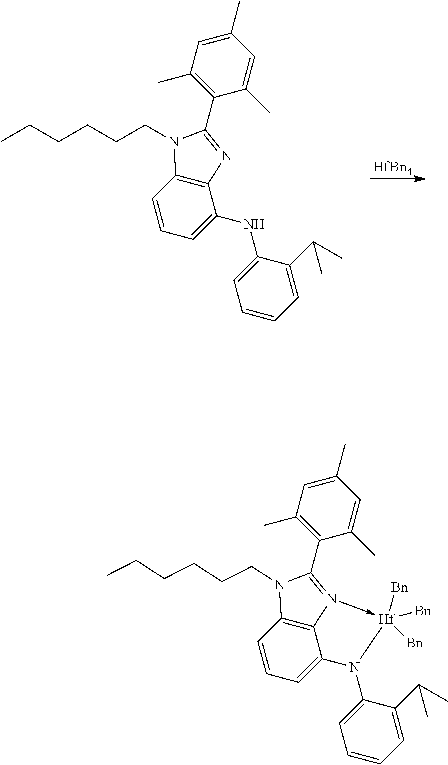

Used General Procedure for Metal Complex Synthesis.

¹H NMR (400 MHz, C₆D₆) δ 7.41-7.31 (m, 1H), 7.19-7.07 (m, 6H), 7.07-7.01 (m, 6H), 7.00 (d, J=1.5 Hz, 1H), 6.88-6.80 (m, 3H), 6.75 (d, J=1.6 Hz, 1H), 6.62 (d, J=1.7 Hz, 1H), 6.56-6.49 (m, 6H), 6.45-6.39 (m, 1H), 6.04 (d, J=7.8 Hz, 1H), 3.55 (hept, J=6.8 Hz, 1H), 3.40 (tt, J=9.2, 7.0 Hz, 2H), 2.31-2.13 (m, 6H), 2.11 (s, 3H), 2.03 (s, 3H), 1.85 (s, 3H), 1.45-1.29 (m, 2H), 1.21 (d, J=6.9 Hz, 3H), 1.15 (d, J=6.8 Hz, 3H), 1.06-0.95 (m, 2H), 0.94-0.80 (m, 4H), 0.75 (t, J=7.2 Hz, 3H).

¹³C NMR (101 MHz, C₆D₆) δ 151.99, 147.04, 147.02, 145.52, 144.85, 141.00, 138.57, 138.40, 138.01, 137.52, 132.11, 131.97, 129.92, 129.27, 129.07, 128.97, 128.88, 128.60, 128.31, 128.20, 127.64, 126.92, 126.88, 126.44, 126.23, 125.33, 124.76, 124.38, 121.90, 105.75, 98.39, 84.92, 44.65, 30.96, 29.11, 27.82, 26.22, 24.94, 23.67, 22.20, 20.78, 20.19, 19.94, 13.72.

### Example 39—Synthesis of Inventive Metal-Ligand Complex 2 (IMLC 2)

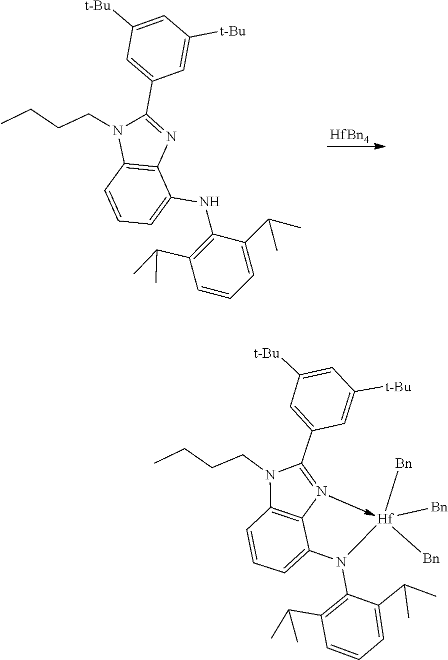

Used General Procedure for Metal Complex Synthesis.

¹H NMR (400 MHz, C₆D₆) δ 7.70 (t, J=1.8 Hz, 1H), 7.48 (d, J=1.8 Hz, 2H), 7.30 (d, J=1.9 Hz, 3H), 7.15-6.90 (m, 14H), 6.79 (tt, J=7.3, 1.3 Hz, 3H), 6.68-6.59 (m, 6H), 6.53 (dd, J=8.1, 1.5 Hz, 1H), 6.39 (dd, J=8.1, 0.7 Hz, 1H), 5.97 (dd, J=7.9, 0.7 Hz, 1H), 3.44 (t, J=7.4 Hz, 2H), 3.39 (q, J=6.8 Hz, 1H), 2.34 (s, 6H), 1.36-1.23 (m, 2H), 1.20 (s, 18H), 1.10 (d, J=6.7 Hz, 6H), 0.82 (h, J=7.4 Hz, 2H), 0.51 (t, J=7.3 Hz, 3H).

¹³C NMR (101 MHz, C₆D₆) δ 153.32, 152.16, 146.94, 145.28, 145.20, 131.81, 131.45, 129.92, 128.96, 128.60, 128.26, 128.19, 127.34, 126.54, 126.22, 125.33, 124.65, 124.44, 124.38, 121.73, 106.66, 98.33, 44.48, 34.85, 31.42, 30.95, 28.86, 25.83, 23.89, 19.48, 13.13.

### Example 40—Synthesis of Inventive Metal-Ligand Complex 3 (IMLC 3)

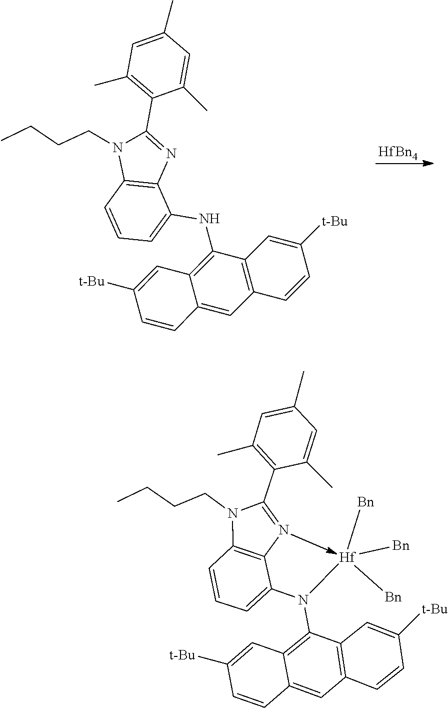

Used General Procedure for Metal Complex Synthesis.

¹H NMR (400 MHz, C₆D₆) δ 8.68-8.62 (m, 2H), 8.20 (s, 1H), 7.94 (d, J=9.0 Hz, 2H), 7.51 (dd, J=8.9, 1.9 Hz, 2H), 7.14-7.05 (m, 7H), 7.05-6.99 (m, 3H), 6.98-6.92 (m, 3H), 6.89 (t, J=7.7 Hz, 6H), 6.84 (t, J=8.0 Hz, 2H), 6.79 (s, 2H), 6.76-6.68 (m, 3H), 6.57-6.49 (m, 4H), 6.44 (d, J=8.0 Hz, 1H), 6.26-6.16 (m, 6H), 5.92 (d, J=7.7 Hz, 1H), 3.50-3.36 (m, 2H), 2.23 (s, 6H), 2.10 (s, 3H), 2.06 (s, 6H), 1.33 (s, 18H), 1.02 (q, J=7.5 Hz, 4H), 0.78-0.71 (m, 3H).

¹³C NMR (101 MHz, C₆D₆) δ 152.56, 148.03, 146.32, 145.81, 141.90, 141.09, 138.56, 138.38, 132.52, 131.97, 131.25, 129.92, 129.04, 129.02, 128.96, 128.61, 128.20, 127.87, 127.53, 126.74, 125.33, 124.44, 124.38, 124.06, 121.67, 118.26, 106.79, 99.14, 88.88, 83.07, 44.68, 34.91, 30.94, 29.06, 26.21, 22.21, 20.82, 19.91, 13.69.

### Example 41—Synthesis of Inventive Metal-Ligand Complex 4 (IMLC 4)

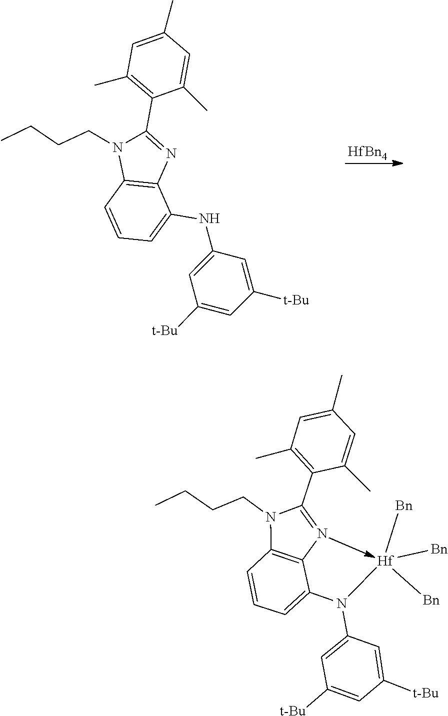

Used General Procedure for Metal Complex Synthesis.

¹H NMR (400 MHz, C₆D₆) δ 7.46 (t, J=1.8 Hz, 1H), 7.23 (d, J=1.8 Hz, 2H), 7.15-7.04 (m, 12H), 7.01 (ddq, J=7.4, 1.4, 0.8 Hz, 2H), 6.97-6.92 (m, 1H), 6.87-6.80 (m, 3H), 6.68 (dd, J=8.3, 1.3 Hz, 8H), 6.55-6.51 (m, 2H), 6.49 (dd, J=8.1, 0.7 Hz, 1H), 6.45 (dd, J=7.8, 0.7 Hz, 1H), 3.37 (dd, J=9.0, 6.5 Hz, 2H), 2.29 (s, 6H), 1.99 (s, 3H), 1.87 (s, 6H), 1.36 (s, 18H), 1.06-0.96 (m, 2H), 0.94-0.79 (m, 4H), 0.74 (t, J=7.2 Hz, 3H).

¹³C NMR (101 MHz, C₆D₆) δ 152.46, 152.18, 148.67, 147.16, 144.83, 140.90, 138.56, 138.06, 137.52, 133.07, 131.80, 129.92, 128.96, 128.82, 128.61, 128.27, 128.19, 127.79, 126.65, 125.33, 124.81, 124.38, 122.68, 121.86, 118.48, 105.01, 98.11, 85.50, 44.49, 34.81, 31.44, 30.95, 29.01, 26.17, 22.21, 20.74, 19.88, 13.70.

### Example 42—Synthesis of Inventive Metal-Ligand Complex 5 (IMLC 5)

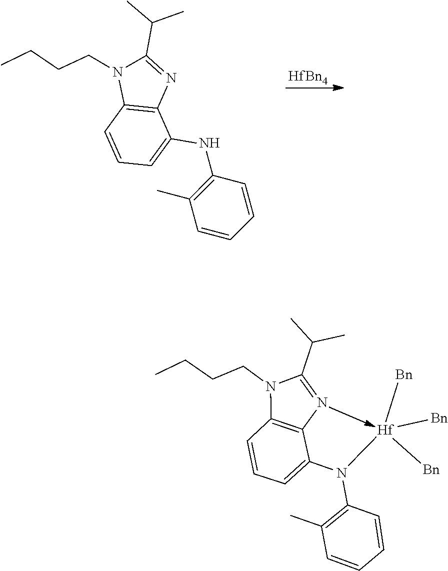

Used General Procedure for Metal Complex Synthesis.

¹H NMR (400 MHz, C₆D₆) δ 7.24-6.98 (m, 21H), 6.98-6.86 (m, 4H), 6.82-6.74 (m, 9H), 6.63-6.57 (m, 1H), 6.56-6.50 (m, 5H), 6.26 (d, J=8.1 Hz, 1H), 5.82 (d, J=7.8 Hz, 1H), 3.44 (dd, J=9.4, 7.0 Hz, 2H), 3.11 (hept, J=7.3 Hz, 1H), 2.45 (d, J=2.3 Hz, 6H), 1.95 (s, 3H), 1.42 (p, J=7.9 Hz, 2H), 1.06 (d, J=7.3 Hz, 6H), 0.98 (h, J=7.4 Hz, 2H), 0.69 (t, J=7.4 Hz, 3H).

¹³C NMR (101 MHz, C₆D₆) δ 157.14, 147.47, 144.90, 144.78, 138.57, 137.53, 135.46, 132.65, 131.04, 130.60, 130.57, 129.93, 128.97, 128.77, 128.60, 128.35, 128.20, 127.36, 127.11, 125.87, 125.76, 125.33, 124.38, 121.74, 104.47, 98.00, 86.14, 44.56, 31.56, 29.17, 21.08, 20.15, 19.81, 17.42, 13.30.

### Example 43—Synthesis of Inventive Metal-Ligand Complex 9 (IMLC 9)

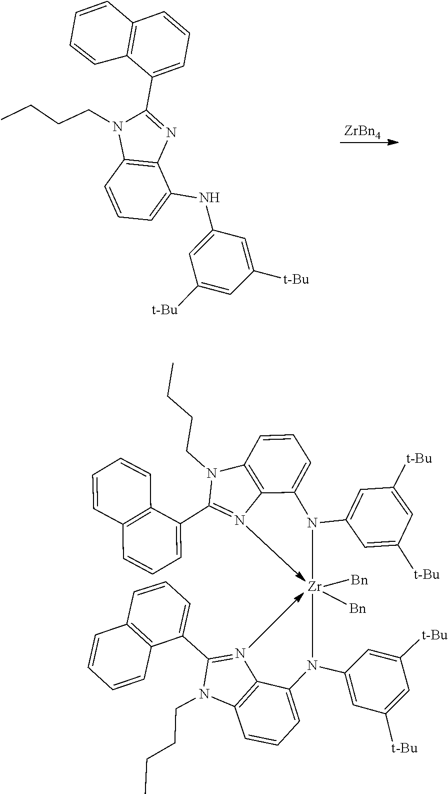

Used General Procedure for Metal Complex Synthesis.

¹H NMR was too complicated to assign. Hindered rotation would require variable temperature NMR. No ¹³C NMR due to low signal to noise.

### Example 44—Synthesis of Inventive Metal-Ligand Complex 6 (IMLC 6)

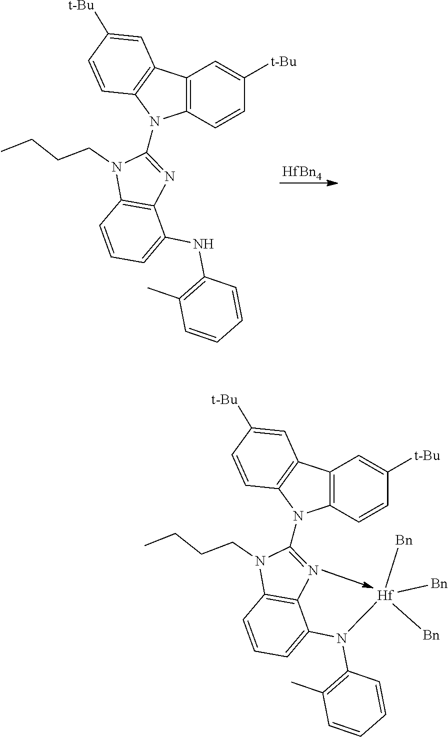

Used General Procedure for Metal Complex Synthesis.

¹H NMR (400 MHz, C₆D₆) δ 8.38-8.27 (m, 2H), 7.41-6.98 (m, 16H), 6.94 (q, J=7.6 Hz, 8H), 6.75 (t, J=7.3 Hz, 3H), 6.63 (dd, J=7.7, 1.3 Hz, 1H), 6.56-6.48 (m, 4H), 6.49-6.43 (m, 6H), 6.03 (d, J=7.9 Hz, 1H), 3.24 (t, J=7.4 Hz, 2H), 2.17 (s, 6H), 2.13 (s, 3H), 1.30 (s, 9H), 1.25 (s, 9H), 1.17 (dq, J=13.4, 6.7, 5.8 Hz, 2H), 0.70-0.55 (m, 2H), 0.31 (t, J=7.3 Hz, 3H).

¹³C NMR (101 MHz, C₆D₆) δ 147.61, 146.45, 145.45, 144.12, 142.01, 139.48, 139.28, 138.54, 137.48, 135.35, 131.82, 131.24, 131.03, 130.56, 129.87, 128.92, 128.90, 128.56, 128.29, 128.15, 125.88, 125.28, 124.95, 124.33, 121.93, 116.95, 110.02, 105.28, 98.25, 85.10, 44.10, 34.44, 31.83, 31.76, 31.59, 31.48, 31.43, 30.98, 19.27, 17.61, 12.72.

### Example 45—Synthesis of Inventive Metal-Ligand Complex 8 (IMLC 8)

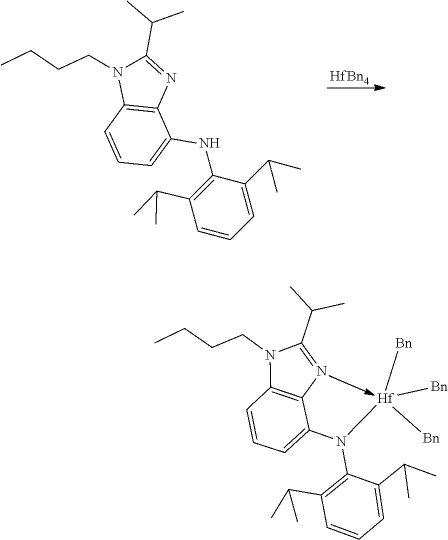

Used General Procedure for Metal Complex Synthesis.

¹H NMR (400 MHz, C₆D₆) δ 7.20-6.98 (m, 13H), 6.97-6.75 (m, 11H), 6.25 (dd, J=8.1, 0.7 Hz, 1H), 5.86 (dd, J=7.9, 0.7 Hz, 1H), 3.50-3.40 (m, 2H), 3.29 (p, J=6.8 Hz, 2H), 3.04 (p, J=7.3 Hz, 1H), 2.95-2.13 (br s, 6H), 1.40-1.28 (m, 2H), 1.21 (d, J=6.9 Hz, 6H), 1.04 (d, J=6.7 Hz, 6H), 0.96 (d, J=7.3 Hz, 6H), 0.95-0.87 (m, 2H), 0.65 (t, J=7.3 Hz, 3H).

¹³C NMR (101 MHz, C₆D₆) δ 157.54, 145.99, 145.46, 144.40, 138.56, 137.53, 132.85, 130.59, 130.25, 129.92, 128.96, 128.60, 128.46, 128.19, 127.27, 126.64, 125.51, 125.32, 124.44, 121.84, 106.26, 98.31, 44.82, 31.40, 29.32, 28.64, 25.88, 24.07, 20.04, 19.81, 13.26.

### Example 46—Synthesis of Inventive Metal-Ligand Complex 7 (IMLC 7)

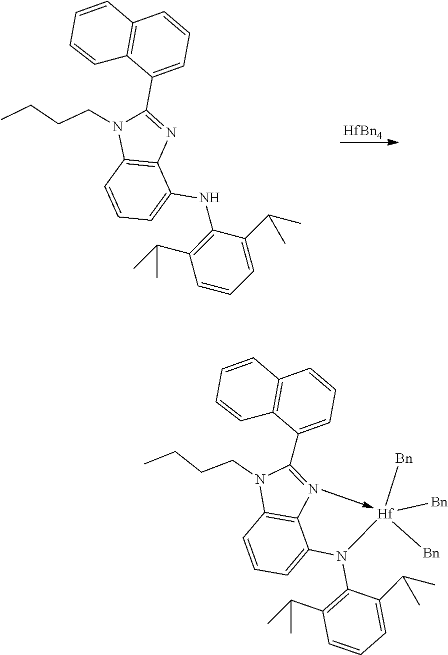

Used General Procedure for Metal Complex Synthesis.

¹H NMR (400 MHz, C₆D₆) δ 7.60 (d, J=8.2 Hz, 1H), 7.53-7.44 (m, 2H), 7.34-7.25 (m, 3H), 7.14-6.99 (m, 10H), 6.95 (t, J=7.6 Hz, 6H), 6.81-6.72 (m, 3H), 6.55 (d, J=7.6 Hz, 6H), 6.46 (d, J=8.1 Hz, 1H), 6.03 (d, J=7.8 Hz, 1H), 3.65 (hept, J=6.9 Hz, 1H), 3.38 (ddd, J=13.8, 8.0, 5.5 Hz, 1H), 3.27 (p, J=6.7 Hz, 1H), 3.19 (dt, J=14.1, 7.7 Hz, 1H), 2.26 (s, 6H), 1.34-1.28 (m, 2H), 1.26 (d, J=6.9 Hz, 3H), 1.16 (d, J=6.7 Hz, 3H), 1.11 (d, J=6.8 Hz, 3H), 1.08 (d, J=6.7 Hz, 3H), 0.78-0.53 (m, 2H), 0.37 (t, J=7.3 Hz, 3H).

¹³C NMR (101 MHz, C₆D₆) δ 151.32, 146.97, 145.57, 145.19, 144.89, 137.53, 133.41, 132.07, 131.88, 131.33, 129.94, 128.96, 128.71, 128.60, 128.37, 128.22, 128.20, 127.36, 126.92, 126.62, 126.52, 125.41, 125.33, 125.17, 124.62, 124.41, 124.32, 121.74, 106.76, 98.45, 44.68, 31.17, 28.88, 28.72, 26.03, 25.91, 24.03, 23.82, 19.24, 12.97.

### Example 47—Synthesis of Inventive Metal-Ligand Complex 10 (IMLC 10)

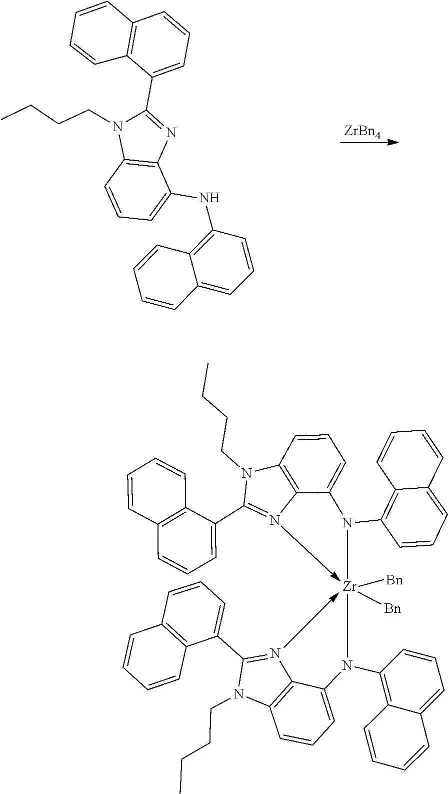

Used General Procedure for Metal Complex Synthesis.

¹H NMR (400 MHz, C₆D₆) δ 8.31-8.18 (m, 1H), 7.78-7.69 (m, 2H), 7.69-7.18 (m, 20H), 7.13-6.82 (m, 32H), 6.79 (t, J=7.2 Hz, 6H), 6.51 (d, J=8.1 Hz, 2H), 6.43-6.33 (m, 8H), 6.29 (d, J=7.6 Hz, 5H), 5.93 (dd, J=12.0, 7.7 Hz, 2H), 3.44 (tt, J=14.7, 6.7 Hz, 2H), 3.26 (q, J=7.0 Hz, 4H), 2.25 (d, J=5.2 Hz, 4H), 2.21 (s, 2H), 2.19 (s, 2H), 2.13 (s, 2H), 1.37 (dddd, J=13.6, 9.1, 7.7, 6.0 Hz, 2H), 1.29-1.13 (m, 2H), 0.90-0.60 (m, 4H), 0.46 (t, J=7.0 Hz, 6H).

¹³C NMR (101 MHz, C₆D₆) δ 147.57, 144.24, 137.53, 131.92, 131.26, 130.56, 130.22, 128.35, 128.20, 126.89, 126.73, 126.26, 126.11, 125.95, 125.33, 124.97, 124.79, 124.46, 124.10, 122.10, 121.94, 105.26, 104.77, 97.78, 76.18, 75.43, 72.13, 44.49, 31.55, 31.29, 21.07, 19.32, 13.08.

### Example 48—Synthesis of Inventive Metal-Ligand Complex 11 (IMLC 11)

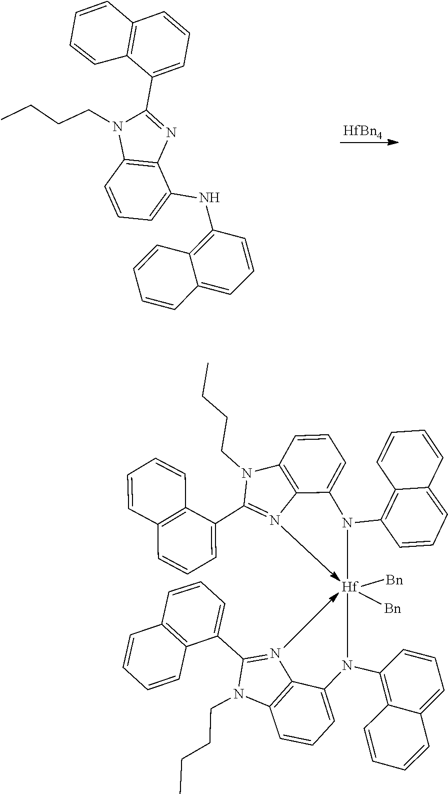

Used General Procedure for Metal Complex Synthesis.

¹H NMR (400 MHz, C₆D₆) δ 7.91 (d, J=8.3 Hz, 1H), 7.73 (dd, J=8.6, 4.4 Hz, 2H), 7.69-7.17 (m, 19H), 7.13-7.08 (m, 6H), 7.08-6.85 (m, 19H), 6.83-6.71 (m, 5H), 5.87 (t, J=8.4 Hz, 1H), 3.43 (ddd, J=13.9, 7.9, 5.8 Hz, 2H), 3.27 (dtd, J=14.0, 6.9, 3.8 Hz, 2H), 2.19-2.12 (m, 8H), 1.29 (dddt, J=60.0, 22.7, 15.6, 7.2 Hz, 4H), 0.91-0.58 (m, 4H), 0.46 (dd, J=20.0, 7.3 Hz, 6H).

¹³C NMR (101 MHz, C₆D₆) δ 151.11, 145.82, 144.57, 137.53, 135.26, 135.17, 133.51, 131.88, 131.48, 131.41, 128.97, 128.23, 128.20, 128.18, 127.56, 127.09, 126.95, 126.44, 126.12, 125.82, 125.71, 125.33, 125.19, 125.04, 124.87, 124.39, 121.69, 121.61, 106.01, 105.74, 98.22, 85.89, 44.72, 44.57, 31.53, 31.21, 19.36, 13.09.

### Example 49—Synthesis of Inventive Metal-Ligand Complex 12 (IMLC 12)

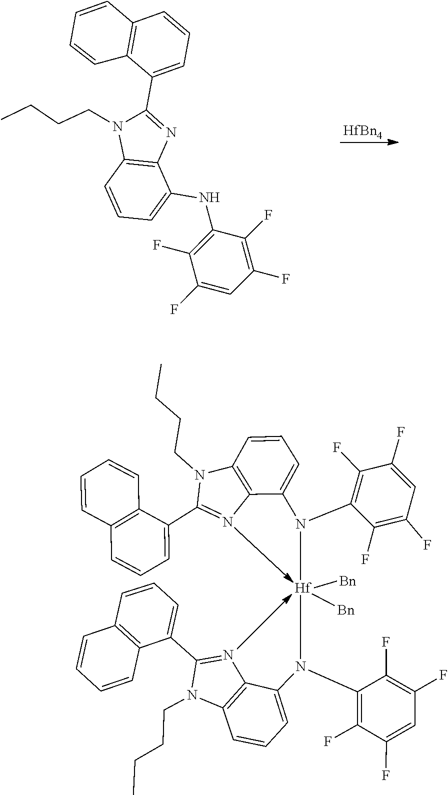

Used General Procedure for Metal Complex Synthesis.

¹H NMR was too complicated to assign. Hindered rotation would require variable temperature NMR.

¹H NMR (400 MHz, C₆D₆) δ 7.75-7.25 (m, 8H), 7.14-6.90 (m, 13H), 6.75 (td, J=7.3, 1.3 Hz, 2H), 6.60-6.42 (m, 5H), 6.33-6.18 (m, 2H), 3.32-3.22 (m, 2H), 3.22-3.01 (m, 2H), 2.25-2.12 (m, 2H), 2.10 (s, 6H), 1.31-1.15 (m, 1H), 1.09-0.91 (m, 2H), 0.72-0.46 (m, 3H), 0.46-0.23 (in, 6H).

¹³C NMR (101 MHz, C₆D₆) δ 137.53, 131.97, 131.53, 129.82, 128.96, 126.95, 125.32, 122.14, 109.17, 104.97, 99.64, 88.42, 65.54, 44.63, 31.16, 21.05, 19.27, 15.22, 12.98.

¹⁹F NMR (376 MHz, C₆D₆) 8-139.81, −139.84, −139.87, −139.88, −139.91, −139.93, −140.21, −141.01, −142.05, −143.40, −145.81, −147.07, −147.13, −148.89, −151.69.

### Example 50—Synthesis of Inventive Metal-Ligand Complex 13 (IMLC 13)

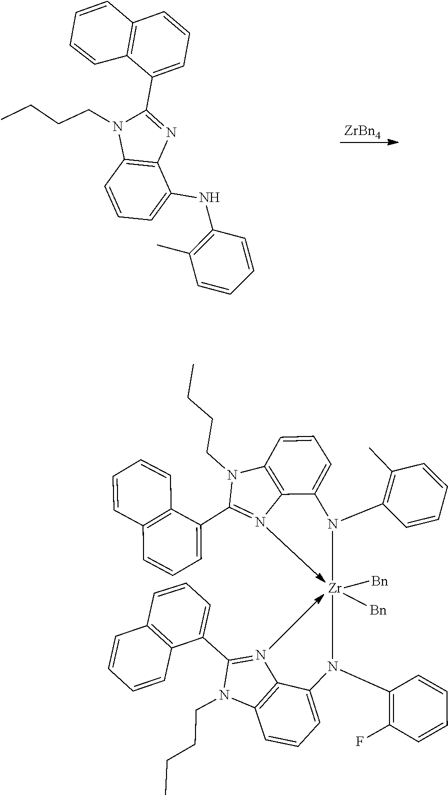

Used General Procedure for Metal Complex Synthesis.

¹H NMR was too complicated to assign. Hindered rotation would require variable temperature NMR. Signal to noise was too low to get a good ¹³C NMR.

¹H NMR (400 MHz, C₆D₆) δ 7.72-7.20 (m, 10H), 7.13-6.86 (m, 16H), 6.83-6.32 (m, 12H), 6.31-5.59 (m, 5H), 3.66-3.05 (m, 4H), 2.79-2.43 (m, 2H), 2.40-2.14 (m, 5H), 2.11 (s, 6H), 1.81-0.87 (m, 7H), 0.84-0.52 (m, 4H), 0.52-0.16 (m, 6H).

### Example 51—Synthesis of 7-bromo-3,3-dimethyl-2-phenyl-3H-indole

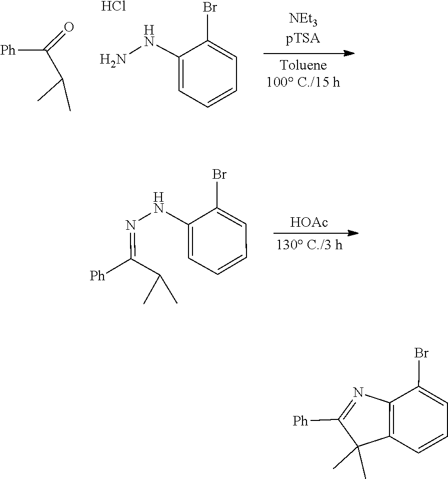 [SMILES: Cl ; CC(C)C(=O)c1ccccc1 ; NNc1ccccc1Br ; CC(C)/C(=N\Nc1ccccc1Br)c1ccccc1 ; CC1(C)C(c2ccccc2)=Nc2c(Br)cccc21 ; "HOAc"]

Step 1: A 20 mL vial was charged with (2-bromophenyl)hydrazine-HCl (6.86 g, 33.7 mmol), toluene (100 mL), NEt₃ (4.70 mL, 30.7 mmol), 2,4-dimethylpentan-3-one (5.00 g, 3.7 mmol), and pTSA (20 mg, catalytic). Reaction was heated to 100° C. for 15 h. Aqueous K₂CO₃ and EtOAc were added and the organic layer was collected and dried over Na₂SO₄. Solids were filtered off and all volatiles were removed to yield a yellow oil. A crude NMR showed the desired product and some starting material. The mixture was used without further purification.

Step 2: Glacial acetic acid was added to the crude mixture from the previous step after water wash and heated to 120° C. for 3 h. A crude LC-MS of the product showed the Fisher-Indole product was formed cleanly. The reaction was cooled to room temperature then ether and water were added and the organic layer was collected. All volatiles were removed and the crude product was purified by column chromatography (80:20 Hex:EtOAc). Yield: 0.45 g, 23%.

1H NMR (400 MHz, CDCl₃) δ 8.24-8.18 (m, 2H), 7.59-7.48 (m, 4H), 7.31-7.26 (m, 1H), 7.15 (dd, J=8.0, 7.3 Hz, 1H), 1.62 (s, 6H).

¹³C NMR (101 MHz, CDCl₃) δ 184.24, 151.47, 149.30, 132.94, 131.19, 130.93, 128.64, 128.61, 127.16, 119.96, 115.03, 55.33, 24.73.

### Example 52—Synthesis of 3-Bromo-N-butyl-2-nitroaniline

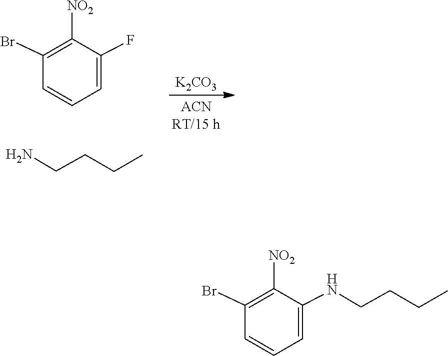

A 250-mL round-bottom was charged with 1-bromo-3-fluoro-2-nitrobenzene (10.00 g, 45.45 mmol), K₂CO₃ (7.54 g, 54.55 mmol), and acetonitrile (100 mL). n-BuNH2 (4.5 mL, 45.45 mmol) was added and the reaction was stirred for 2 d at room temperature. All volatiles were removed and the crude product was taken up in EtOAc and water. The organic layer was collected and dried over Na₂SO₄. Solids were filtered off and all volatiles were removed to yield the product as an orange solid/oil. The NMR indicates a 75:25 ratio of product to starting material. The material was used on the next step without further purification. Yield: 12.20 g, 98%.

¹H NMR (400 MHz, CDCl₃) δ 7.15 (dd, J=8.5, 7.8 Hz, 1H), 6.94 (dd, J=7.8, 1.1 Hz, 1H), 6.76 (dd, J=8.6, 1.1 Hz, 1H), 5.73 (s, 1H), 3.20 (td, J=7.1, 5.1 Hz, 2H), 1.66 (tt, J=8.6, 6.8 Hz, 2H), 1.52-1.39 (m, 2H), 0.98 (t, J=7.3 Hz, 3H).

¹³C NMR (101 MHz, CDCl₃) δ 143.83, 132.99, 121.39, 116.29, 112.38, 43.23, 31.00, 20.14, 13.76.

### Example 53—Synthesis of 3-bromo-N¹-butylbenzene-1,2-diamine

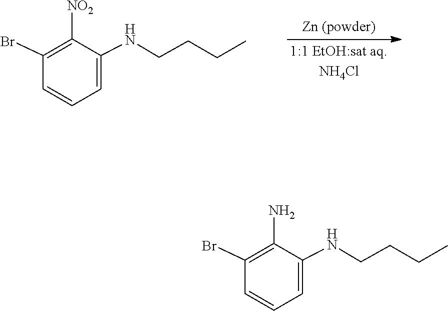

A 100 mL round bottom was charged with the 3-bromo-N-butyl-2-nitroaniline (2.64 g, 9.67 mmol), ethanol (30 mL), and sat. aq. NH₄Cl (10 mL). The mixture was stirred at room temperature under nitrogen, then Zn powder (5.06 g, 77.33 mmol)) was added in portions. The reaction was monitored by LC-MS. After stirring for 2 h EtOAc was added and the mixture was filtered through Celite. The organic layer was collected and purified by column chromatography (80:20 Hex:EtOAc). Yield: 1.72 g, 73%.

¹H NMR (400 MHz, CDCl₃) δ 6.95 (dd, J=8.1, 1.3 Hz, 1H), 6.70 (t, J=8.0 Hz, 1H), 6.65-6.58 (m, 1H), 3.76 (s, 2H), 3.35 (s, 1H), 3.12 (td, J=7.0, 3.6 Hz, 2H), 1.68 (dtd, J=8.6, 7.3, 5.9 Hz, 2H), 1.56-1.42 (m, 2H), 1.00 (t, J=7.3 Hz, 3H).

¹³C NMR (101 MHz, CDCl₃) δ 138.89, 132.35, 121.60, 120.82, 111.23, 110.41, 44.14, 31.71, 20.41, 13.95.

### Example 54—Synthesis of 4-Bromo-1-butyl-2-(2-methylphenyl)-1H-benzo[d]imidazole

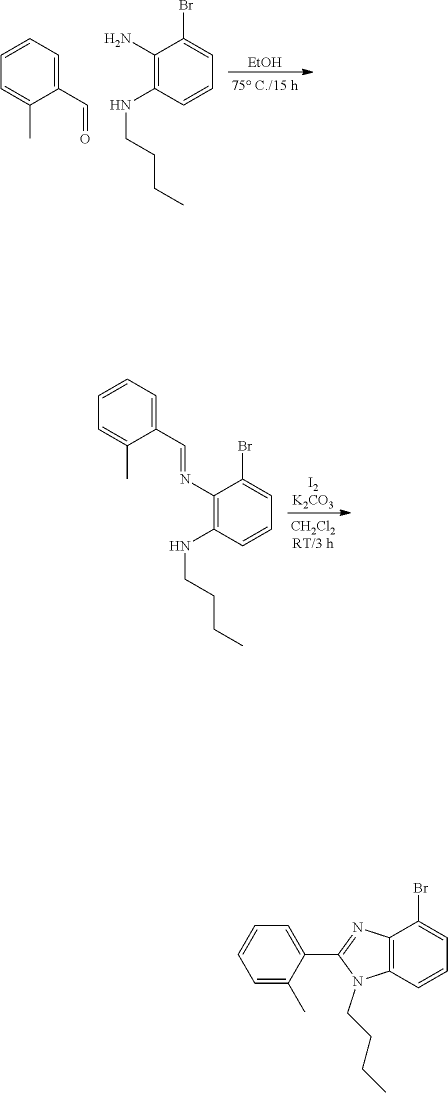

A 250-mL round-bottom flask was charged with 3-bromo-N¹-butylbenzene-1,2-diamine (2.08 g, 8.55 mmol), 2-methylbenzaldehyde (0.99 mL, 8.55 mmol), and EtOH (100 mL, absolute). The mixture was heated to 70° C. for 15 h. All volatiles were removed, then CH₂Cl₂ (100 mL), K₂CO₃ (2.60 g, 18.8 mmol), and 12 (2.17 g, 8.55 mmol) were added and the mixture was allowed to stir for 3 h. Water was added to the mixture and the organic layer was collected. The crude product was purified by column chromatography (60:40 Hex:EtOAc, 2nd product). Yield: 2.21 g, 75%.

¹H NMR (400 MHz, CDCl₃): δ 7.50 (dd, J=7.7, 0.9 Hz, 1H), 7.44-7.35 (m, 3H), 7.35-7.29 (m, 2H), 7.18 (t, J=7.9 Hz, 1H), 4.04-3.93 (m, 2H), 2.24 (s, 3H), 1.72-1.58 (m, 2H), 1.18 (h, J=7.4 Hz, 2H), 0.79 (t, J=7.3 Hz, 3H).

¹³C NMR (101 MHz, CDCl₃) δ 154.06, 141.89, 138.03, 135.15, 130.40, 130.25, 129.99, 129.86, 125.69, 125.18, 123.33, 113.45, 109.35, 44.35, 31.54, 19.77, 19.76, 13.46.

### Example 55—Synthesis of 4-Bromo-1-butyl-2-(4-tertbutylphenyl)-1H-benzo[d]imidazole

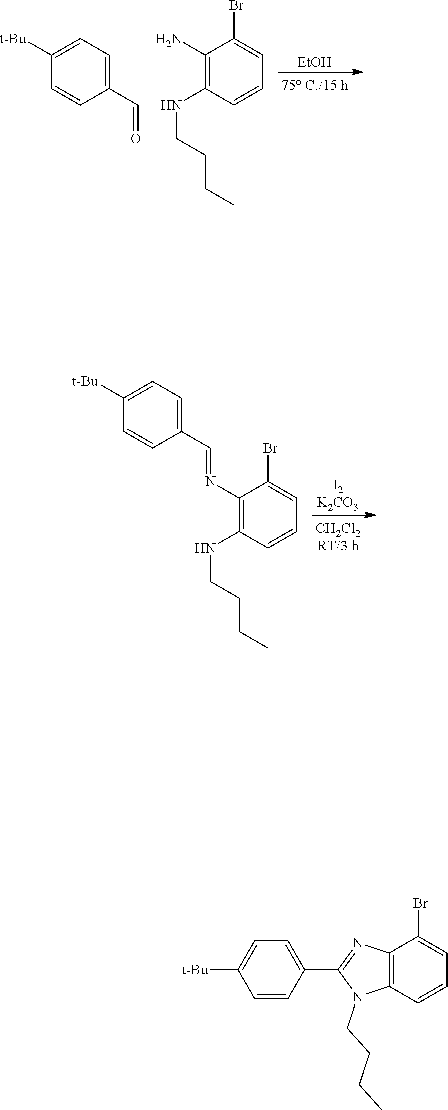

A 250-mL round-bottom flask was charged with 3-bromo-N¹-butylbenzene-1,2-diamine (1.00 g, 4.11 mmol), 4-tertbutylbenzaldehyde (0.69 mL, 4.11 mmol), and EtOH (100 mL, absolute). The mixture was heated to 70° C. for 15 h. All volatiles were removed, then CH₂Cl₂ (100 mL), K₂CO₃ (1.25 g, 9.05 mmol), and 12 (1.04 g, 4.11 mmol) were added and the mixture was allowed to stir for 3 h. Water was added to the mixture and the organic layer was collected. The crude product was purified by column chromatography (60:40 Hex:EtOAc, 2nd product). Yield: 1.11 g, 70%.

¹H NMR (400 MHz, CDCl₃) δ 7.59-7.52 (m, 2H), 7.47-7.41 (m, 2H), 7.37 (dd, J=7.8, 0.9 Hz, 1H), 7.24 (dd, J=8.1, 0.9 Hz, 1H), 7.03 (t, J=7.9 Hz, 1H), 4.13-4.07 (m, 2H), 1.74-1.59 (m, 2H), 1.30 (s, 9H), 1.24-1.10 (m, 2H), 0.77 (t, J=7.4 Hz, 3H).

¹³C NMR (101 MHz, CDCl₃) δ 154.45, 153.06, 141.84, 136.05, 129.20, 127.20, 125.57, 125.08, 123.24, 113.16, 109.46, 44.78, 34.79, 31.72, 31.21, 19.82, 13.51.

### Example 56—Synthesis of 4-Bromo-1-butyl-1,3-dihydro-2H-benzo[d]imidazol-2-one

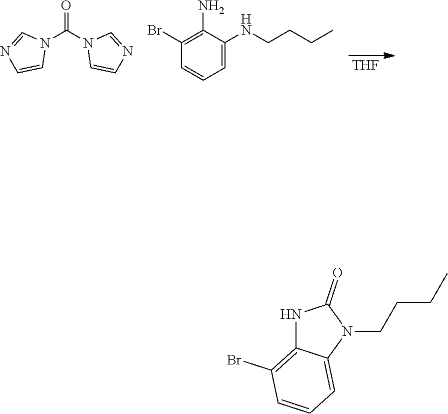

A 20-mL vial was charged with 3-bromo-N′-butylbenzene-1,2-diamine (0.589 g, 2.42 mmol) and THF (10 mL, not anhydrous). 1,1′-Carbonyldimidazole (0.393 g, 2.42 mmol) was added and the mixture was heated to 55° C. for 15 h. All volatiles were removed and the crude product was purified by column chromatography (Hex:EtOAc 60:40) to yield the pure product. Yield: 0.493 g, 76%.

¹H NMR (400 MHz, CDCl3) δ 9.45 (s, 1H), 7.20 (dd, J=7.9, 1.2 Hz, 1H), 6.99 (t, J=7.9 Hz, 1H), 6.94 (dt, J=7.9, 1.0 Hz, 1H), 3.90 (t, J=7.2 Hz, 2H), 1.89-1.68 (m, 2H), 1.60-1.28 (m, 2H), 0.98 (t, J=7.4 Hz, 3H).

¹³C NMR (101 MHz, CDCl3) δ 154.61, 131.17, 127.63, 123.95, 122.34, 106.76, 102.29, 41.02, 30.37, 20.05, 13.72.

### Example 57—Synthesis of 4-Bromo-1-butyl-2-chloro-1H-benzo[d]imidazole

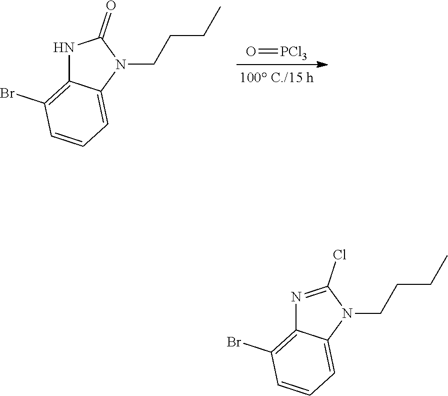

A 20-mL vial was charged with the 4-bromo-1-butyl-1,3-dihydro-2H-benzo[d]imidazol-2-one (0.493 g, 1.83 mmol) and POCl₃ (2.05 mL, 21.98 mmol). The neat mixture was heated under nitrogen at 100° C. overnight. The reaction was cooled and CH₂Cl₂ (8 mL) was added then water was added slowly (quenching was sluggish at first, but became very fast with time). The organic layer was collected and dried over Na₂SO₄. The solids were filtered off and all volatiles were removed. The crude product looked good by NMR. No further purification was needed. Yield: 0.498 g, 95%.

¹H NMR (400 MHz, CDCl₃) δ 7.34 (dd, J=7.8, 0.9 Hz, 1H), 7.18 (dd, J=8.1, 0.9 Hz, 1H), 7.06 (t, J=8.0 Hz, 1H), 4.08 (t, J=7.3 Hz, 2H), 1.69 (dq, J=9.2, 7.3 Hz, 2H), 1.37-1.19 (m, 2H), 0.86 (t, J=7.4 Hz, 3H).

¹³C NMR (101 MHz, CDCl₃) δ 141.07, 139.59, 135.04, 125.90, 124.23, 112.02, 109.06, 44.91, 31.18, 19.82, 13.55.

### Example 58—Synthesis of 9-(4-Bromo-1-butyl-1H-benzo[d]imidazol-2-yl)-3,6-di-tert-butyl-9H-carbazole

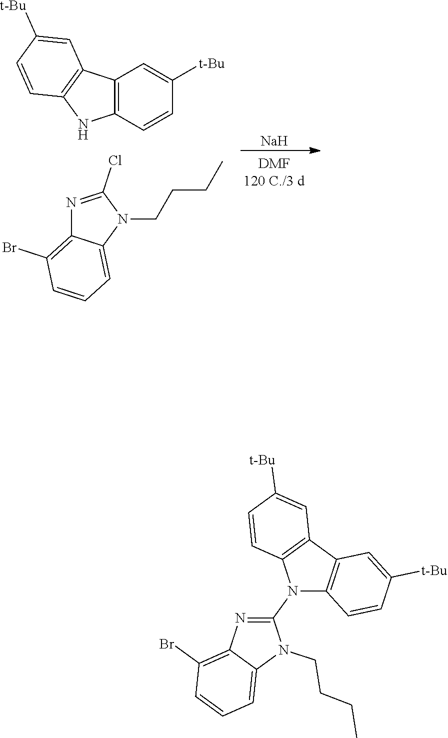

A 20-mL vial was charged with NaH (0.031 g, 1.31 mmol) in the glovebox. The vial was taken out of the glovebox and a DMF (6 mL) solution of the 3,6-di-tert-butyl-9H-carbazole (Cbz, 0.365 g, 1.31 mmol) and 4-bromo-1-butyl-2-chloro-1H-benzo[d]imidazole (0.365 g, 0.65 mmol) were added to the vial. The vial was heated to 120° C. over the weekend. Hexanes and water were added and the organic layer was collected. All volatiles were removed and the crude product was purified by column chromatography (Hex:EtOAc 90:10). The product and starting Cbz nearly co-elute. Yield: 0.064 g, 18%.

¹H NMR (400 MHz, CDCl₃) δ 8.12 (dd, J=2.0, 0.7 Hz, 2H), 7.59 (dd, J=7.8, 0.9 Hz, 1H), 7.52-7.44 (m, 3H), 7.28 (t, J=8.0 Hz, 1H), 7.26 (dd, J=8.5, J=0.6 Hz, 2H), 4.08 (t, J=7.1 Hz, 2H), 1.45 (s, 18H), 1.37-1.25 (m, 2H), 1.06-0.95 (m, 2H), 0.61 (t, J=7.4 Hz, 3H).

¹³C NMR (101 MHz, CDCl₃) δ 145.48, 144.43, 140.93, 138.96, 134.79, 125.79, 124.34, 124.22, 123.88, 116.40, 113.82, 110.25, 109.61, 44.63, 34.81, 31.95, 31.12, 19.56, 13.20.

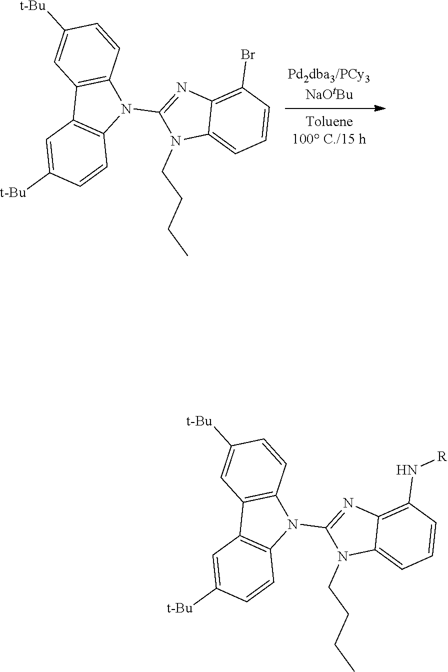

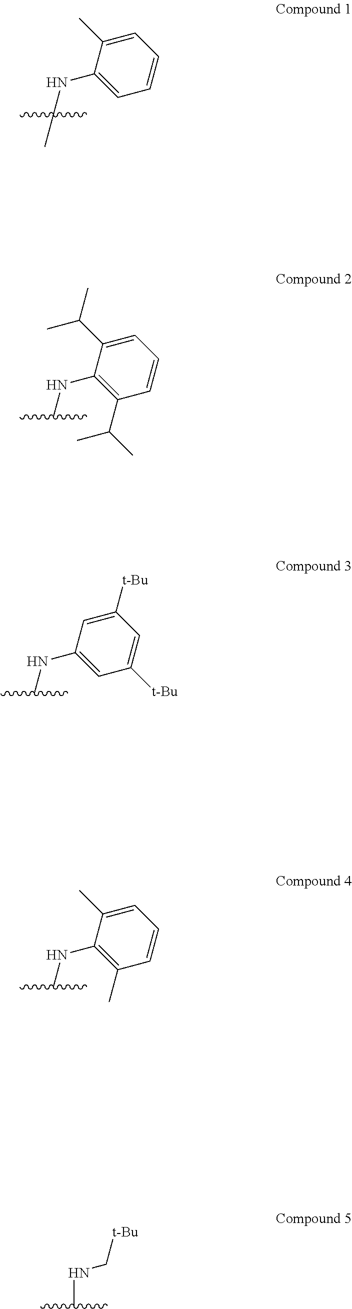

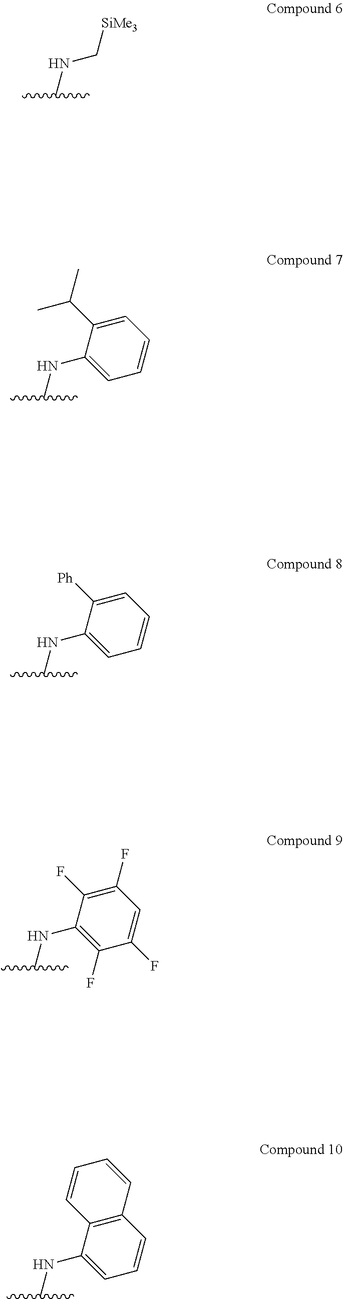 [SMILES: *NC[Si](C)(C)C ; *Nc1ccccc1C(C)C ; *Nc1ccccc1-c1ccccc1 ; *Nc1c(F)c(F)cc(F)c1F ; *Nc1cccc2ccccc12]

Buckwald-Hartwig Coupling for 9-(4-bromo-1-butyl-1H-benzo[d]imidazol-2-yl)-3,6-di-tert-butyl-9H-carbazole:

Buchwald-Hartwig Cross Coupling reaction in a high throughput sequence beginning with CM3 manipulation (Library 75278).

Brominated starting material were provided and reacted with excess amine (2:1). All reactants/reagents were delivered in solution (Toluene) with the exception of sodium t-butoxide and the catalyst (weighed as solids). Reaction were diluted with additional reaction solvent to −10 mL before overnight reaction. The following day reaction conversion was checked via UPLC. After 16 hours at 95° C. conversion was high enough to proceed with purification.

Purification consisted of three phases: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25 mL Biotage ISOLUTE® Phase separator column. An additional 5 mL of chloroform was added and the organic phase was collected after gravity filtration. The collected material was poured into a GL Sciences 20 mL InertSep PS-SL filter and gravity filtered again. One wash of 5 mL chloroform was similarly used to rinse the phase separation column, then InertSep filter. A final rinse of the silica pad was performed with 5 mL ethyl acetate and the collected samples were concentrated over 10 hours at 80° C. under vacuum on a Savant SpeedVac, which ramped at 5 Torr/min. The solids were delivered back to T. Paine for purification on the SFC.

Preparative SFC was used to purify using a 1-AA 130 Å 5 μm OBD 30×150 mm column using CO2 as mobile phase A and 75% acetonitrile:25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL and injection volume was 960 μL. The desired compounds were collected by mass spectrometry.

### Example 59—Compound 1

Yield=0.114 g, 57%.

¹H NMR (400 MHz, CDCl₃) δ 8.16 (d, J=1.9 Hz, 2H), 7.60-7.55 (m, 1H), 7.51 (dd, J=8.6, 1.9 Hz, 2H), 7.31-7.19 (m, 5H), 7.05 (td, J=7.4, 1.3 Hz, 1H), 6.96 (dd, J=8.2, 0.9 Hz, 1H), 6.92 (dd, J=7.9, 0.8 Hz, 1H), 6.65 (s, 1H), 4.04 (t, J=7.2 Hz, 2H), 2.36 (s, 3H), 1.68-1.58 (m, 2H), 1.50 (s, 18H), 1.15-0.99 (m, 2H), 0.65 (t, J=7.3 Hz, 3H).

¹³C NMR (101 MHz, CDCl₃) δ 144.16, 142.23, 139.42, 138.72, 137.77, 136.67, 134.76, 130.41, 128.43, 126.06, 124.33, 124.26, 124.03, 116.43, 110.05, 102.57, 99.38, 44.21, 34.83, 31.99, 31.36, 19.76, 18.42, 13.29.

### Example 60—Compound 2

Yield=0.092 g, 46%.

¹H NMR (400 MHz, CDCl₃) δ 8.20-8.10 (m, 2H), 7.57-7.49 (m, 2H), 7.40-7.22 (m, 6H), 7.11 (t, J=8.0 Hz, 1H), 6.84 (dd, J=8.1, 0.9 Hz, 1H), 6.35 (s, 1H), 6.03 (dd, J=8.0, 0.9 Hz, 1H), 4.04 (t, J=7.3 Hz, 2H), 3.36 (hept, J=6.8 Hz, 2H), 1.50 (s, 18H), 1.19 (d, J=6.9 Hz, 12H), 1.10 (dt, J=14.4, 7.2 Hz, 2H), 0.66 (t, J=7.3 Hz, 3H).

¹³C NMR (101 MHz, CDCl₃) δ 148.12, 144.14, 142.10, 140.58, 139.40, 134.71, 134.64, 130.01, 127.40, 124.33, 124.29, 124.01, 123.78, 116.44, 110.06, 102.58, 98.91, 44.22, 34.83, 32.00, 31.49, 31.37, 28.19, 19.77, 13.29.

### Example 61—Compound 4

Yield=0.059 g, 29%.

¹H NMR (400 MHz, CDCl₃) δ 8.16 (d, J=1.9 Hz, 2H), 7.57-7.49 (m, 2H), 7.33-7.26 (m, 3H), 7.20-7.11 (m, 4H), 6.87 (dd, J=8.1, 0.9 Hz, 1H), 6.40 (s, 1H), 6.06 (dd, J=7.9, 0.9 Hz, 1H), 4.04 (t, J=7.3 Hz, 2H), 2.33 (s, 6H), 1.70-1.59 (m, 2H), 1.50 (s, 18H), 1.17-1.03 (m, 2H), 0.66 (t, J=7.3 Hz, 3H).

¹³C NMR (101 MHz, CDCl₃) δ 144.16, 142.23, 139.42, 138.72, 137.77, 136.67, 134.76, 130.40, 128.43, 126.05, 124.33, 124.26, 124.03, 116.43, 110.05, 102.57, 99.38, 44.21, 34.83, 31.99, 31.35, 19.75, 18.42, 13.29.

### Example 62—Compound 5

Yield=0.099 g, 49%.

¹H NMR (400 MHz, CDCl₃) δ 8.14 (d, J=1.9 Hz, 2H), 7.49 (dd, J=8.6, 1.9 Hz, 2H), 7.33-7.14 (m, 4H), 6.79 (dd, J=8.2, 0.8 Hz, 1H), 6.54 (d, J=7.9 Hz, 1H), 5.05 (s, 1H), 3.97 (t, J=7.2 Hz, 2H), 3.14 (s, 2H), 1.65-1.51 (m, 2H), 1.49 (s, 18H), 1.07 (s+m, 9+2H), 0.62 (t, J=7.3 Hz, 3H).

¹³C NMR (101 MHz, CDCl₃) δ 144.04, 141.82, 141.65, 139.39, 134.49, 130.34, 124.57, 124.23, 123.92, 116.37, 110.00, 100.94, 98.13, 55.54, 44.05, 34.81, 32.42, 31.99, 31.28, 27.76, 19.67, 13.25.

### Example 63—Compound 6

Yield=0.081 g, 40%.

¹H NMR (400 MHz, CDCl3) δ 8.14 (d, J=1.8 Hz, 2H), 7.49 (dd, J=8.6, 1.9 Hz, 2H), 7.31 (d, J=8.0 Hz, 1H), 7.24-7.16 (m, 2H), 6.81 (dd, J=8.1, 0.9 Hz, 1H), 6.59 (dd, J=8.0, 0.8 Hz, 1H), 4.84 (s, 1H), 3.95 (t, J=7.2 Hz, 2H), 2.73 (s, 2H), 1.61-1.51 (m, 2H), 1.49 (s, 18H), 1.09-0.96 (m, 2H), 0.61 (t, J=7.4 Hz, 3H), 0.19 (s, 9H).

¹³C NMR (101 MHz, CDCl3) δ 144.01, 143.03, 141.57, 139.39, 134.38, 130.35, 124.64, 124.21, 123.89, 116.36, 110.00, 100.97, 98.33, 44.02, 34.81, 33.27, 31.99, 31.27, 19.65, 13.24, −2.45.

### Example 64—Compound 7

Yield=0.109 g, 55%.

¹H NMR (400 MHz, CDCl₃) δ 8.16 (d, J=1.9 Hz, 2H), 7.53 (ddd, J=8.6, 4.9, 1.7 Hz, 3H), 7.39 (dd, J=7.6, 1.8 Hz, 1H), 7.31-7.26 (m, 3H), 7.26-7.16 (m, 3H), 6.93 (dd, J=8.1, 0.9 Hz, 1H), 6.78-6.72 (m, 1H), 6.65 (s, 1H), 4.04 (t, J=7.2 Hz, 2H), 3.36 (hept, J=6.9 Hz, 1H), 1.68-1.58 (m, 2H), 1.50 (s, 18H), 1.26 (d, J=6.8 Hz, 6H), 1.14-1.02 (m, 2H), 0.65 (t, J=7.3 Hz, 3H).

¹³C NMR (101 MHz, CDCl₃) δ 144.18, 143.05, 142.46, 139.37, 138.44, 138.42, 134.84, 131.22, 126.36, 126.25, 124.60, 124.30, 124.29, 124.13, 124.02, 116.44, 110.06, 103.72, 100.12, 44.21, 34.83, 31.99, 31.32, 27.68, 23.40, 19.71, 13.28.

### Example 65—Compound 8

Yield=0.027 g, 14%.

¹H NMR (400 MHz, CDCl₃) δ 8.17-8.10 (m, 2H), 7.76 (dd, J=8.6, 1.2 Hz, 1H), 7.50 (ddd, J=8.6, 5.6, 1.7 Hz, 4H), 7.42-7.34 (m, 4H), 7.33-7.19 (m, 6H), 7.15 (td, J=7.5, 1.2 Hz, 1H), 6.99 (dd, J=7.7, 1.2 Hz, 1H), 6.80 (s, 1H), 4.03 (t, J=7.2 Hz, 2H), 1.60 (dq, J=9.4, 7.4 Hz, 2H), 1.50 (s, 18H), 1.10-0.96 (m, 2H), 0.63 (t, J=7.4 Hz, 3H).

¹³C NMR (101 MHz, CDCl₃) δ 144.18, 142.73, 139.12, 139.03, 138.98, 136.62, 135.01, 133.90, 132.02, 131.13, 129.26, 128.74, 128.03, 127.33, 124.23, 124.02, 123.92, 122.55, 120.96, 116.39, 110.13, 104.81, 101.23, 44.29, 34.82, 31.99, 31.20, 19.66, 13.25.

### Example 66—Compound 9

Yield=0.125 g, 62%.

¹H NMR (400 MHz, CDCl₃) δ 8.16 (d, J=1.9 Hz, 2H), 7.51 (dd, J=8.6, 1.9 Hz, 2H), 7.36-7.22 (m, 4H), 7.11 (dd, J=8.2, 0.8 Hz, 1H), 6.85 (tt, J=9.9, 7.0 Hz, 1H), 6.77 (s, 1H), 6.65 (dt, J=7.2, 3.1 Hz, 1H), 4.09 (t, J=7.2 Hz, 2H), 1.69-1.56 (m, 2H), 1.13-1.01 (m, 2H), 0.65 (t, J=7.4 Hz, 3H).

¹³C NMR (101 MHz, CDCl₃) δ 144.39, 143.47, 139.19, 134.78, 134.01, 131.98, 124.41, 124.14, 123.73, 116.51, 110.00, 105.91, 103.16, 99.52, 44.39, 34.83, 31.96, 31.27, 19.66, 13.25.

### Example 67—Compound 10

Yield=0.021 g, 11%.

¹H NMR (400 MHz, CDCl₃) δ 8.24-8.19 (m, 1H), 8.17 (d, J=1.9 Hz, 2H), 7.94-7.87 (m, 1H), 7.72 (d, J=7.4 Hz, 1H), 7.67 (d, J=8.2 Hz, 1H), 7.57-7.43 (m, 5H), 7.35-7.19 (m, 5H), 6.99 (dd, J=8.1, 0.9 Hz, 1H), 6.93 (dd, J=7.9, 0.8 Hz, 1H), 4.08 (t, J=7.2 Hz, 2H), 1.70-1.61 (m, 2H), 1.50 (s, 18H), 1.18-1.03 (m, 2H), 0.67 (t, J=7.3 Hz, 3H).

¹³C NMR (101 MHz, CDCl₃) δ 144.28, 142.78, 139.35, 137.63, 137.56, 134.93, 134.75, 131.59, 128.75, 128.35, 126.14, 125.90, 125.72, 124.37, 124.11, 124.09, 123.82, 122.62, 118.07, 116.50, 110.04, 104.88, 100.84, 44.28, 34.84, 31.99, 31.33, 19.71, 13.30.

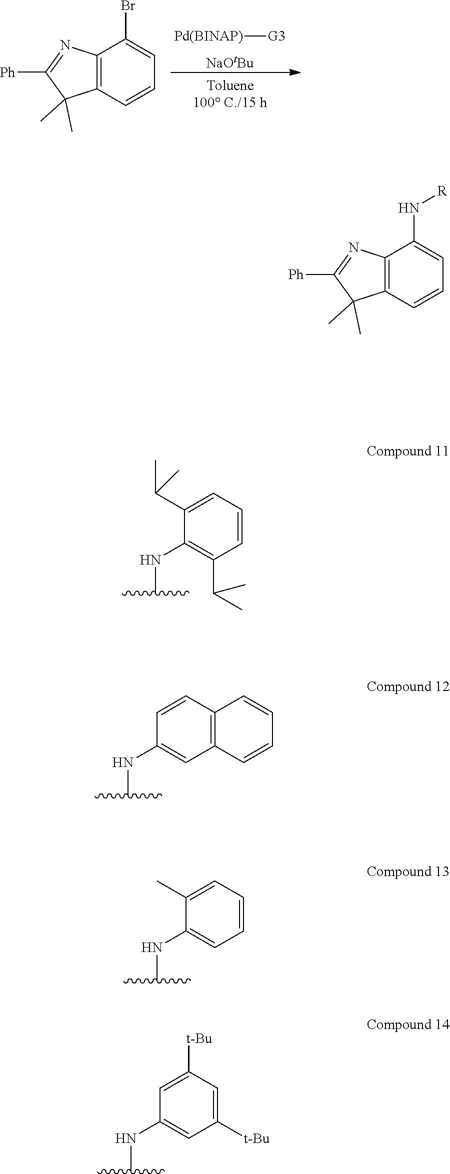

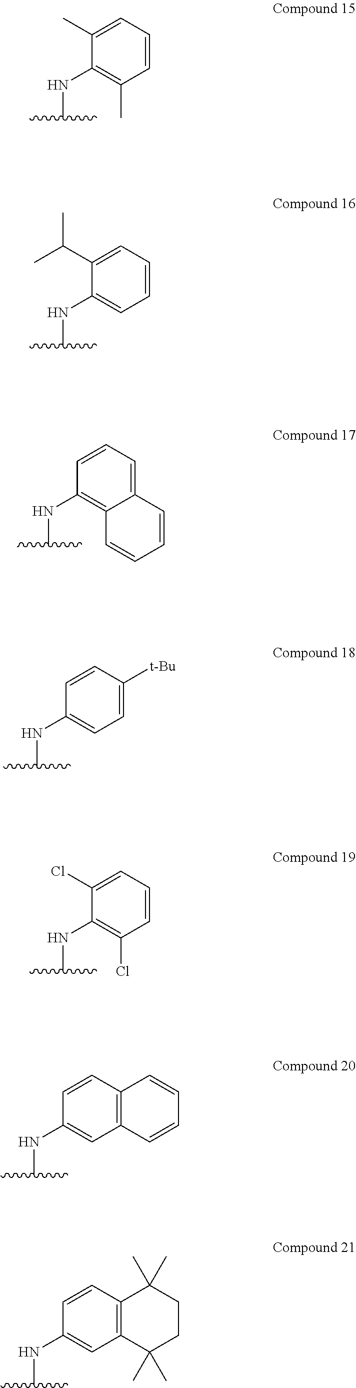

Buckwald-Hartwig Coupling for 7-bromo-3,3-dimethyl-2-phenyl-3H-indole:

The brominated compound and amines were provided for a Buchwald-Hartwig Cross Coupling reaction in a high throughput sequence beginning with CM3 manipulation.

Brominated starting material were provided and reacted with excess amine (2:1). All reactants/reagents were delivered in solution (Toluene) with the exception of sodium t-butoxide and the catalyst (weighed as solids). Reaction were diluted with additional reaction solvent to ˜10 mL before overnight reaction. The following day reaction conversion was checked via UPLC. After 16 hours at 95° C. conversion was high enough to proceed with purification.

Purification consisted of three phases: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25 mL Biotage ISOLUTE® Phase separator column. An additional 5 mL of chloroform was added and the organic phase was collected after gravity filtration. The collected material was poured into a GL Sciences 20 mL InertSep PS-SL filter and gravity filtered again. One wash of 5 mL chloroform was similarly used to rinse the phase separation column, then InertSep filter. A final rinse of the silica pad was performed with 5 mL ethyl acetate and the collected samples were concentrated over 10 hours at 80° C. under vacuum on a Savant SpeedVac, which ramped at 5 Torr/min. The solids were delivered back to T. Paine for purification on the SFC.

Preparative SFC was used to purify using a 1-AA 130 Å 5 μm OBD 30×150 mm column using CO2 as mobile phase A and 75% acetonitrile:25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL and injection volume was 960 μL. The desired compounds were collected by mass spectrometry.

### Example 68—Compound 11

Yield=0.115 g, 81%.

¹H NMR (400 MHz, CDCl₃) δ 8.31-8.18 (m, 2H), 7.54 (qd, J=7.8, 6.8, 3.8 Hz, 3H), 7.44-7.26 (m, 3H), 7.03 (t, J=7.7 Hz, 1H), 6.73 (d, J=7.4 Hz, 1H), 6.50 (s, 1H), 6.17 (d, J=8.1 Hz, 1H), 3.40 (hept, J=6.8 Hz, 2H), 1.68 (s, 6H), 1.24 (d, J=6.9 Hz, 12H).

¹³C NMR (101 MHz, CDCl₃) δ 179.68, 148.31, 147.93, 141.05, 138.69, 135.21, 133.93, 129.99, 128.61, 128.07, 127.31, 127.07, 123.82, 110.10, 109.43, 54.32, 28.29, 25.00, 24.02.

### Example 69—Compound 12

Yield=0.071 g, 59%.

¹H NMR (400 MHz, CDCl₃) δ 8.25 (dt, J=7.3, 1.5 Hz, 2H), 7.85 (dd, J=10.1, 8.1 Hz, 2H), 7.81-7.73 (m, 2H), 7.61-7.44 (m, 6H), 7.44-7.37 (m, 2H), 7.36-7.32 (m, 1H), 7.31-7.25 (m, 1H), 6.94 (dd, J=7.3, 1.0 Hz, 1H), 1.69 (d, J=1.4 Hz, 6H).

¹³C NMR (101 MHz, CDCl₃) δ 180.79, 148.84, 141.11, 139.92, 136.05, 134.66, 133.58, 130.34, 129.55, 129.17, 128.69, 128.66, 128.20, 127.73, 127.06, 126.77, 126.46, 123.76, 121.07, 113.06, 112.71, 112.19, 54.58, 24.87.

### Example 70—Compound 13

Yield=0.095 g, 46%.

¹H NMR (400 MHz, CDCl₃) δ 8.26-8.14 (m, 2H), 7.57-7.47 (m, 4H), 7.30 (d, J=6.4 Hz, 1H), 7.23 (td, J=7.7, 1.6 Hz, 1H), 7.16 (t, J=7.7 Hz, 1H), 7.10-6.98 (m, 2H), 6.88-6.82 (m, 1H), 6.80 (s, 1H), 2.43 (s, 3H), 1.65 (s, 6H).

¹³C NMR (101 MHz, CDCl₃) δ 180.28, 148.69, 140.67, 140.42, 137.08, 133.56, 130.98, 130.20, 129.74, 128.60, 128.13, 126.92, 126.67, 122.55, 119.87, 112.39, 111.33, 54.45, 24.88, 18.11.

### Example 71—Compound 14

Yield=0.086 g, 43%.

¹H NMR (400 MHz, CDCl₃) δ 8.22-8.13 (m, 2H), 7.57-7.46 (m, 3H), 7.30-7.24 (m, 1H), 7.20 (d, J=1.6 Hz, 2H), 7.19-7.15 (m, 1H), 7.12 (t, J=1.7 Hz, 1H), 7.08 (s, 1H), 6.83 (dd, J=7.3, 1.0 Hz, 1H), 1.64 (s, 6H), 1.38 (s, 18H).

¹³C NMR (101 MHz, CDCl₃) δ 180.27, 151.84, 148.65, 141.09, 140.38, 136.98, 133.69, 130.12, 128.60, 128.09, 127.01, 116.20, 114.31, 111.55, 111.04, 54.46, 34.96, 31.50, 24.82.

¹H NMR (400 MHz, CDCl₃) δ 8.22-8.13 (m, 2H), 7.57-7.46 (m, 3H), 7.30-7.24 (m, 1H), 7.20 (d, J=1.6 Hz, 2H), 7.19-7.15 (m, 1H), 7.12 (t, J=1.7 Hz, 1H), 7.08 (s, 1H), 6.83 (dd, J=7.3, 1.0 Hz, 1H), 1.64 (s, 6H), 1.38 (s, 18H).

¹³C NMR (101 MHz, CDCl₃) δ 180.27, 151.84, 148.65, 141.09, 140.38, 136.98, 133.69, 130.12, 128.60, 128.09, 127.01, 116.20, 114.31, 111.55, 111.04, 54.46, 34.96, 31.50, 24.82.

### Example 72—Compound 15

Yield=0.075 g, 38%.

¹H NMR (400 MHz, CDCl₃) δ 8.21 (dt, J=7.8, 2.0 Hz, 2H), 7.52 (tdd, J=7.0, 4.8, 1.9 Hz, 3H), 7.17 (q, J=5.8 Hz, 3H), 7.03 (t, J=7.7 Hz, 1H), 6.73 (dt, J=7.4, 1.3 Hz, 1H), 6.48 (s, 1H), 6.16 (dd, J=8.1, 1.1 Hz, 1H), 2.33 (d, J=2.1 Hz, 6H), 1.65 (s, 6H).

¹³C NMR (101 MHz, CDCl₃) δ 179.84, 148.45, 139.22, 139.06, 138.05, 136.54, 133.81, 130.00, 128.57, 128.46, 128.07, 127.06, 125.95, 110.05, 109.76, 54.27, 24.94, 18.54.

### Example 73—Compound 16

Yield=0.054 g, 27%.

¹H NMR (400 MHz, CDCl₃) δ 8.21 (dtd, J=8.5, 4.3, 2.5 Hz, 2H), 7.57-7.47 (m, 4H), 7.39 (dt, J=7.7, 2.0 Hz, 1H), 7.23 (tt, J=7.7, 2.3 Hz, 1H), 7.14 (tdd, J=8.1, 5.5, 2.0 Hz, 2H), 6.94 (dt, J=8.2, 1.4 Hz, 1H), 6.84 (s, 1H), 6.81 (ddd, J=7.3, 2.1, 1.0 Hz, 1H), 3.39 (pd, J=6.9, 6.5, 1.6 Hz, 1H), 1.66 (d, J=1.8 Hz, 6H), 1.35 (dd, J=6.8, 2.0 Hz, 6H).

¹³C NMR (101 MHz, CDCl₃) δ 180.07, 148.61, 141.55, 140.37, 138.97, 138.15, 133.58, 130.15, 128.60, 128.08, 126.95, 126.36, 126.15, 123.76, 122.30, 111.81, 110.85, 54.43, 27.81, 24.90, 23.18.

### Example 74—Compound 17

Yield=0.055 g, 27%.

¹H NMR (400 MHz, CDCl₃) δ 8.31-8.18 (m, 3H), 7.98-7.86 (m, 1H), 7.66 (d, J=7.8 Hz, 2H), 7.60-7.45 (m, 6H), 7.36 (s, 1H), 7.14 (dd, J=8.2, 7.2 Hz, 1H), 7.06 (dd, J=8.1, 1.1 Hz, 1H), 6.86 (dd, J=7.2, 1.1 Hz, 1H), 1.68 (s, 6H).

¹³C NMR (101 MHz, CDCl₃) δ 180.45, 148.69, 140.74, 137.92, 137.70, 134.78, 133.57, 130.24, 128.63, 128.52, 128.47, 128.16, 126.94, 126.16, 125.97, 125.75, 123.38, 122.34, 116.85, 112.65, 111.47, 54.52, 24.90.

### Example 75—Compound 18

Yield=0.095 g, 47%.

¹H NMR (400 MHz, CDCl₃) δ 8.22-8.13 (m, 2H), 7.51 (qq, J=4.4, 2.7, 1.7 Hz, 3H), 7.42-7.36 (m, 2H), 7.31-7.25 (m, 3H), 7.17 (t, J=7.7 Hz, 1H), 7.03 (s, 1H), 6.83 (dd, J=7.3, 1.1 Hz, 1H), 1.63 (s, 6H), 1.37 (d, J=1.4 Hz, 9H).

¹³C NMR (101 MHz, CDCl₃) δ 180.34, 148.64, 144.69, 140.44, 139.45, 136.80, 133.64, 130.16, 128.61, 128.08, 126.95, 126.10, 119.30, 111.84, 111.26, 54.45, 34.27, 31.52, 24.82.

### Example 76—Compound 19

Yield=0.015 g, 8%.

¹H NMR (400 MHz, CDCl₃) δ 8.26-8.17 (m, 2H), 7.55-7.47 (m, 3H), 7.45 (d, J=8.1 Hz, 2H), 7.12 (dt, J=11.2, 7.9 Hz, 2H), 6.95 (s, 1H), 6.87 (dd, J=7.4, 1.0 Hz, 1H), 6.45-6.36 (m, 1H), 1.65 (s, 6H).

¹³C NMR (101 MHz, CDCl₃) δ 180.58, 148.49, 140.37, 136.40, 136.14, 133.58, 132.43, 130.19, 128.82, 128.54, 128.24, 126.40, 125.90, 112.20, 112.11, 54.35, 24.87.

### Example 77—Compound 20

Yield=0.094 g, 47%.

¹H NMR (400 MHz, CDCl₃) δ 8.26-8.16 (m, 2H), 7.86-7.70 (m, 4H), 7.57-7.50 (m, 3H), 7.50-7.43 (m, 3H), 7.37 (ddd, J=8.1, 6.8, 1.2 Hz, 1H), 7.27-7.22 (m, 2H), 6.91 (dd, J=7.3, 0.9 Hz, 1H), 1.66 (s, 6H).

¹³C NMR (101 MHz, CDCl₃) δ 180.79, 148.79, 141.04, 139.87, 135.99, 134.61, 133.53, 130.31, 129.50, 129.13, 128.66, 128.16, 127.69, 127.01, 126.73, 126.42, 123.71, 121.04, 113.00, 112.66, 112.15, 54.56, 24.83.

### Example 78—Compound 21

Yield=0.025 g, 13%.

¹H NMR (400 MHz, CDCl₃) δ 8.23-8.09 (m, 2H), 7.56-7.45 (m, 3H), 7.30 (d, J=8.4 Hz, 1H), 7.27-7.22 (m, 2H), 7.20-7.09 (m, 2H), 7.01 (s, 1H), 6.81 (dd, J=7.2, 1.0 Hz, 1H), 1.73 (d, J=14.3 Hz, 4H), 1.63 (s, 6H), 1.33 (d, J=2.2 Hz, 12H).

¹³C NMR (101 MHz, CDCl₃) δ 180.23, 148.62, 145.90, 140.30, 139.23, 138.65, 137.01, 133.69, 130.11, 128.60, 128.08, 127.31, 126.98, 117.69, 117.63, 111.58, 110.98, 54.45, 35.23, 34.41, 33.86, 31.96, 31.90, 24.82.

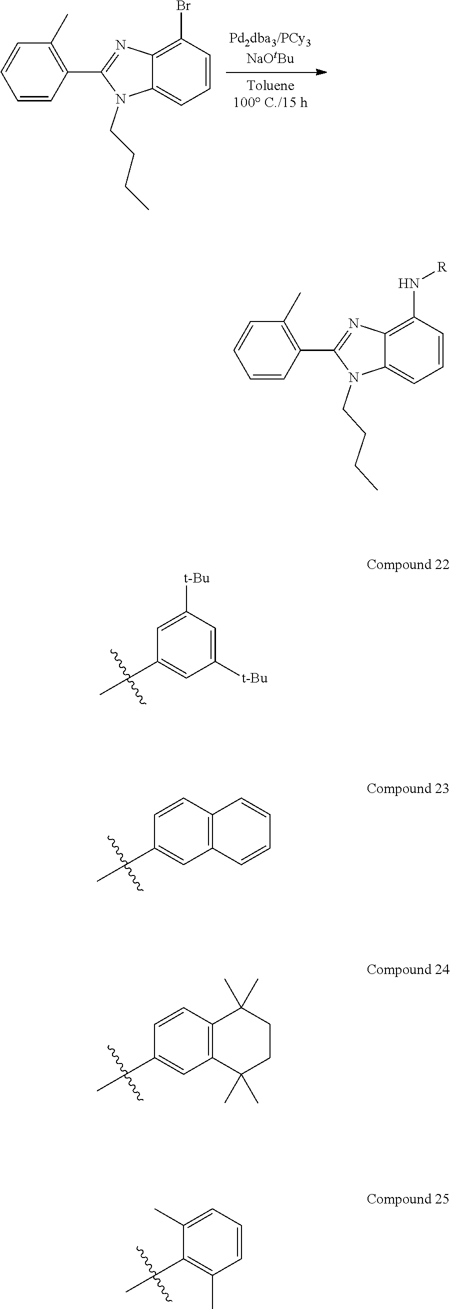

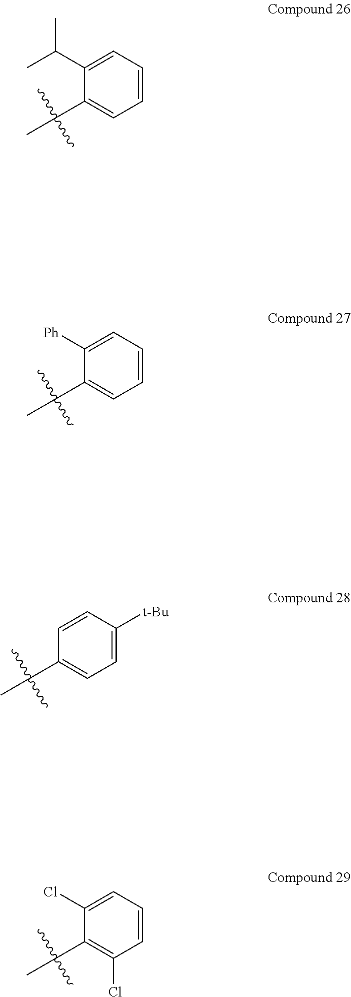

Buckwald-Hartwig Coupling for 4-bromo-1-butyl-2-(o-tolyl)-1H-benzo[d]imidazole:

The brominated compound and amines were provided for a Buchwald-Hartwig Cross Coupling reaction in a high throughput sequence beginning with CM3 manipulation.

Brominated starting material were provided and reacted with excess amine (2:1). All reactants/reagents were delivered in solution (Toluene) with the exception of sodium t-butoxide and the catalyst (weighed as solids). Reaction were diluted with additional reaction solvent to ˜10 mL before overnight reaction. The following day reaction conversion was checked via UPLC. After 16 hours at 95° C. conversion was high enough to proceed with purification.

Purification consisted of three phases: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25 mL Biotage ISOLUTE® Phase separator column. An additional 5 mL of chloroform was added and the organic phase was collected after gravity filtration. The collected material was poured into a GL Sciences 20 mL InertSep PS-SL filter and gravity filtered again. One wash of 5 mL chloroform was similarly used to rinse the phase separation column, then InertSep filter. A final rinse of the silica pad was performed with 5 mL ethyl acetate and the collected samples were concentrated over 10 hours at 80° C. under vacuum on a Savant SpeedVac, which ramped at 5 Torr/min. The solids were delivered back to T. Paine for purification on the SFC.

Preparative SFC was used to purify using a 1-AA 130 Å 5 μm OBD 30×150 mm column using CO2 as mobile phase A and 75% acetonitrile:25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL and injection volume was 960 μL. The desired compounds were collected by mass spectrometry.

### Example 79—Compound 22

Yield=0.072 g, 38%.

¹H NMR (400 MHz, CDCl₃) δ 7.49-7.31 (m, 4H), 7.26-7.17 (m, 4H), 7.09 (dd, J=4.0, 2.2 Hz, 2H), 6.91 (dd, J=7.2, 1.7 Hz, 1H), 4.00 (t, J=7.4 Hz, 2H), 2.29 (s, 3H), 1.79-1.65 (m, 2H), 1.36 (s, 18H), 1.29-1.17 (m, 2H), 0.83 (t, J=7.3 Hz, 3H).

¹³C NMR (101 MHz, CDCl₃) δ 151.72, 151.21, 141.23, 138.15, 136.08, 135.14, 132.72, 130.52, 130.42, 130.31, 129.82, 125.85, 123.52, 115.83, 113.89, 103.63, 100.51, 44.13, 34.94, 31.66, 31.49, 19.85, 19.76, 13.52.

### Example 80—Compound 23

Yield=0.012 g, 6%.

¹H NMR (400 MHz, CDCl₃) δ 7.84-7.71 (m, 4H), 7.50-7.42 (m, 4H), 7.42-7.24 (m, 7H), 7.00 (dd, J=8.0, 0.9 Hz, 1H), 4.02 (t, J=7.4 Hz, 2H), 2.30 (s, 3H), 1.80-1.65 (m, 2H), 1.32-1.17 (m, 2H), 0.84 (t, J=7.4 Hz, 3H).

¹³C NMR (101 MHz, CDCl₃) δ 151.51, 139.93, 138.14, 135.25, 135.19, 134.61, 130.59, 130.29, 129.95, 129.40, 129.02, 127.65, 126.72, 126.34, 125.91, 123.59, 123.48, 121.01, 112.78, 105.00, 101.60, 44.19, 31.64, 19.85, 19.78, 13.52.

### Example 81—Compound 24

Yield=0.095 g, 50%.

¹H NMR (400 MHz, CDCl₃) δ 7.49-7.32 (m, 4H), 7.32-7.25 (m, 3H), 7.24-7.12 (m, 3H), 7.03 (s, 1H), 6.90 (dd, J=7.7, 1.2 Hz, 1H), 3.99 (t, J=7.4 Hz, 2H), 2.28 (s, 3H), 1.78-1.63 (m, 2+4H), 1.32 (d, J=1.7 Hz, 12H), 1.28-1.18 (m, 2H), 0.83 (t, J=7.4 Hz, 3H).

¹³C NMR (101 MHz, CDCl₃) δ 151.17, 145.78, 139.35, 138.26, 138.15, 136.13, 135.13, 132.60, 130.52, 130.39, 130.31, 129.83, 127.23, 125.85, 123.50, 117.42, 117.15, 103.63, 100.43, 44.12, 35.25, 35.21, 34.38, 33.82, 31.95, 31.89, 31.65, 19.86, 19.77, 13.52.

### Example 82—Compound 25

Yield=0.036 g, 19%.

¹H NMR (400 MHz, CDCl₃) δ 7.53-7.29 (m, 6H), 7.25-7.10 (m, 3H), 7.07 (t, J=7.9 Hz, 1H), 6.82 (d, J=8.0 Hz, 1H), 6.45 (s, 1H), 6.01 (d, J=7.8 Hz, 1H), 3.99 (t, J=7.4 Hz, 2H), 2.32 (s, 3+6H), 1.82-1.66 (m, 2H), 1.25 (h, J=7.5 Hz, 2H), 0.84 (t, J=7.4 Hz, 3H).

¹³C NMR (101 MHz, CDCl₃) δ 150.91, 138.43, 138.20, 138.08, 136.58, 135.07, 131.68, 130.59, 130.50, 130.42, 129.74, 128.40, 125.88, 125.80, 123.54, 102.25, 99.29, 44.14, 31.69, 19.91, 19.83, 18.42, 13.53.

### Example 83—Compound 26

Yield=0.101 g, 53%.

¹H NMR (400 MHz, CDCl3) δ 7.52 (dd, J=7.8, 1.5 Hz, 1H), 7.48-7.31 (m, 5H), 7.26-7.09 (m, 3H), 6.87 (dd, J=8.1, 0.9 Hz, 1H), 6.73-6.64 (m, 2H), 4.00 (t, J=7.4 Hz, 2H), 3.39 (hept, J=6.9 Hz, 1H), 2.31 (s, 3H), 1.77-1.64 (m, 2H), 1.27 (d, J=6.9 Hz, 6H), 1.25-1.17 (m, 2H), 0.83 (t, J=7.4 Hz, 3H).

¹³C NMR (101 MHz, CDCl3) δ 151.16, 142.86, 138.68, 138.22, 138.15, 135.16, 132.51, 130.55, 130.51, 130.37, 129.77, 126.30, 126.19, 125.82, 124.35, 124.17, 123.42, 103.44, 100.01, 44.14, 31.65, 27.63, 23.42, 19.87, 19.85, 13.51.

### Example 84—Compound 27

Yield=0.099 g, 52%.

¹H NMR (400 MHz, CDCl₃) δ 7.76-7.69 (m, 1H), 7.53-7.48 (m, 2H), 7.44-7.26 (m, 10H), 7.25-7.17 (m, 2H), 7.12 (td, J=7.5, 1.2 Hz, 1H), 6.93 (dd, J=6.3, 2.6 Hz, 1H), 6.75 (s, 1H), 3.98 (t, J=7.4 Hz, 2H), 2.21 (s, 3H), 1.77-1.62 (m, 2H), 1.29-1.16 (m, 2H), 0.82 (t, J=7.4 Hz, 3H).

¹³C NMR (101 MHz, CDCl₃) δ 151.23, 139.20, 139.11, 138.25, 136.35, 135.36, 133.67, 133.30, 131.12, 130.55, 130.28, 130.19, 129.69, 129.32, 128.67, 127.95, 127.28, 125.70, 123.25, 122.34, 120.95, 104.51, 101.09, 44.11, 31.65, 19.86, 19.79, 13.51.

### Example 85—Compound 28

Yield=0.056 g, 30%.

¹H NMR (400 MHz, CDCl₃) δ 7.49-7.31 (m, 6H), 7.31-7.26 (m, 3H), 7.19 (dd, J=8.1, 3.7 Hz, 2H), 7.05 (s, 1H), 6.91 (dd, J=6.4, 2.6 Hz, 1H), 3.99 (t, J=7.4 Hz, 2H), 2.28 (s, 3H), 1.77-1.63 (m, 2H), 1.36 (s, 9H), 1.23 (dd, J=6.6, 1.8 Hz, 2H), 0.82 (t, J=7.3 Hz, 3H).

¹³C NMR (101 MHz, CDCl₃) δ 151.22, 144.38, 139.49, 138.15, 136.00, 135.15, 130.53, 130.34, 130.30, 129.85, 126.03, 125.85, 123.45, 119.09, 103.86, 100.66, 44.13, 34.23, 31.64, 31.50, 19.85, 19.77, 13.51.

### Example 86—Compound 29

Yield=0.018 g, 9%.

¹H NMR (400 MHz, CDCl₃) δ 7.44 (dd, J=7.8, 6.4 Hz, 4H), 7.41-7.31 (m, 2H), 7.13 (td, J=8.0, 5.2 Hz, 2H), 6.96 (d, J=8.0 Hz, 1H), 6.92 (s, 1H), 6.27 (d, J=7.8 Hz, 1H), 4.00 (t, J=7.4 Hz, 2H), 2.31 (s, 3H), 1.78-1.65 (m, 2H), 1.30-1.16 (m, 2H), 0.83 (t, J=7.3 Hz, 3H).

¹³C NMR (101 MHz, CDCl₃) δ 151.54, 138.22, 136.48, 135.45, 135.19, 132.61, 132.60, 130.51, 130.41, 130.39, 129.78, 128.81, 125.92, 125.77, 122.87, 104.73, 101.65, 44.17, 31.66, 19.88, 19.85, 13.51.

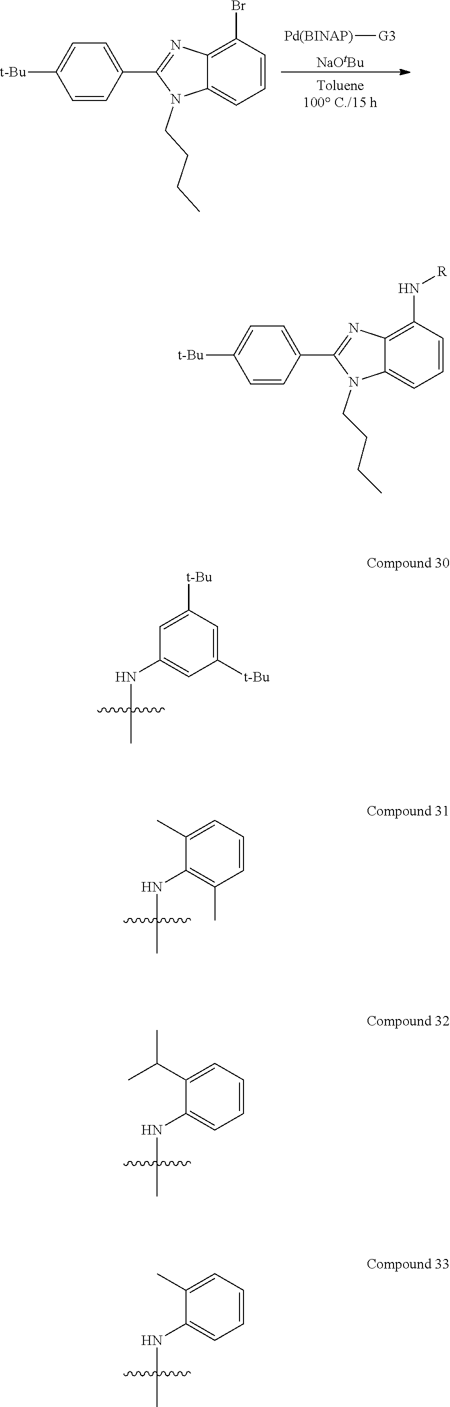

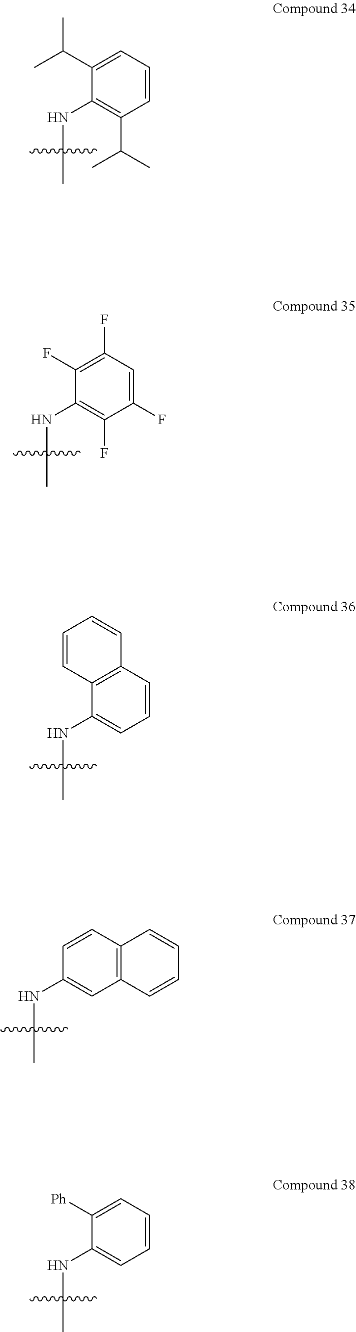 [SMILES: *Nc1c(C(C)C)cccc1C(C)C ; *Nc1c(F)c(F)cc(F)c1F ; *Nc1cccc2ccccc12 ; *Nc1ccc2ccccc2c1 ; *Nc1ccccc1-c1ccccc1]

Buckwald-Hartwig Coupling for 4-bromo-1-butyl-2-(4-(tert-butyl)phenyl)-1H-benzo[d]imidazole:

The brominated compound and amines were provided for a Buchwald-Hartwig Cross Coupling reaction in a high throughput sequence beginning with CM3 manipulation.

Brominated starting material were provided and reacted with excess amine (2:1). All reactants/reagents were delivered in solution (Toluene) with the exception of sodium t-butoxide and the catalyst (weighed as solids). Reaction were diluted with additional reaction solvent to −10 mL before overnight reaction. The following day reaction conversion was checked via UPLC. After 16 hours at 95° C. conversion was high enough to proceed with purification.

Purification consisted of three phases: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25 mL Biotage ISOLUTE® Phase separator column. An additional 5 mL of chloroform was added and the organic phase was collected after gravity filtration. The collected material was poured into a GL Sciences 20 mL InertSep PS-SL filter and gravity filtered again. One wash of 5 mL chloroform was similarly used to rinse the phase separation column, then InertSep filter. A final rinse of the silica pad was performed with 5 mL ethyl acetate and the collected samples were concentrated over 10 hours at 80° C. under vacuum on a Savant SpeedVac, which ramped at 5 Torr/min. The solids were delivered back to T. Paine for purification on the SFC.

Preparative SFC was used to purify using a 1-AA 130 Å 5 μm OBD 30×150 mm column using CO2 as mobile phase A and 75% acetonitrile:25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL and injection volume was 960 μL. The desired compounds were collected by mass spectrometry.

### Example 87—Compound 30

Yield=0.066 g, 55%.

¹H NMR (400 MHz, CDCl₃) δ 7.69-7.52 (m, 4H), 7.25-7.14 (m, 4H), 7.14-7.06 (m, 2H), 6.90 (dd, J=7.4, 1.5 Hz, 1H), 4.29-4.18 (m, 2H), 1.94-1.79 (m, 2H), 1.41 (s, 9H), 1.37 (s, 18H), 1.33-1.29 (m, 2H), 0.92 (t, J=7.3 Hz, 3H).

¹³C NMR (101 MHz, CDCl₃) δ 152.77, 151.85, 151.68, 141.30, 136.10, 136.02, 132.84, 129.01, 127.95, 125.78, 123.56, 115.81, 113.99, 103.71, 100.53, 44.65, 34.94, 34.87, 31.98, 31.50, 31.27, 20.00, 13.62.

### Example 88—Compound 31

Yield=0.073 g, 61%.

¹H NMR (400 MHz, CDCl₃) δ 7.71-7.53 (m, 4H), 7.21-7.10 (m, 3H), 7.04 (t, J=7.9 Hz, 1H), 6.80 (dd, J=8.1, 0.9 Hz, 1H), 6.46 (s, 1H), 5.98 (dd, J=7.7, 0.8 Hz, 1H), 4.28-4.15 (m, 2H), 2.31 (s, 6H), 1.96-1.82 (m, 2H), 1.41 (s, 9H), 1.35 (dd, J=14.9, 7.4 Hz, 2H), 0.93 (t, J=7.4 Hz, 3H).

¹³C NMR (101 MHz, CDCl₃) δ 152.65, 151.54, 138.36, 138.08, 136.61, 135.99, 131.74, 129.09, 128.39, 128.08, 125.87, 125.72, 123.62, 102.27, 99.30, 44.69, 34.85, 32.04, 31.28, 20.07, 18.43, 13.64.

### Example 89—Compound 32

Yield=0.076 g, 63%.

¹H NMR (400 MHz, CDCl₃) δ 7.63 (ddd, J=44.8, 8.4, 1.5 Hz, 4H), 7.51 (dd, J=7.8, 1.6 Hz, 1H), 7.39 (dt, J=7.6, 1.6 Hz, 1H), 7.27-7.08 (m, 3H), 6.89-6.83 (m, 1H), 6.72 (s, 1H), 6.68 (d, J=7.8 Hz, 1H), 4.23 (t, J=7.7 Hz, 2H), 3.39 (hept, J=6.8 Hz, 1H), 1.97-1.80 (m, 2H), 1.41 (s, 9H), 1.35 (p, J=7.5 Hz, 2H), 1.27 (dd, J=6.8, 1.3 Hz, 6H), 0.93 (td, J=7.4, 1.4 Hz, 3H).

¹³C NMR (101 MHz, CDCl₃) δ 152.73, 151.79, 142.71, 138.74, 138.02, 136.10, 132.62, 129.07, 127.99, 126.30, 126.16, 125.74, 124.25, 123.98, 123.49, 103.59, 100.05, 44.70, 34.86, 32.00, 31.28, 27.64, 23.40, 20.04, 13.63.

### Example 90—Compound 33

Yield=0.069 g, 58%.

1H NMR (400 MHz, CDCl3) δ 7.72-7.65 (m, 2H), 7.60-7.51 (m, 3H), 7.27-7.12 (m, 3H), 7.02 (td, J=7.4, 1.3 Hz, 1H), 6.93-6.88 (m, 1H), 6.86 (d, J=7.8 Hz, 1H), 6.70 (s, 1H), 4.28-4.17 (m, 2H), 2.38 (s, 3H), 1.95-1.80 (m, 2H), 1.41 (s, 9H), 1.40-1.29 (m, 2H), 0.92 (t, J=7.4 Hz, 3H).

¹³C NMR (101 MHz, CDCl3) δ 152.78, 151.94, 140.35, 136.58, 136.17, 132.96, 130.94, 130.34, 129.07, 127.93, 126.56, 125.77, 125.76, 123.40, 122.74, 121.00, 104.38, 100.63, 44.70, 34.87, 31.98, 31.28, 20.02, 18.10, 13.62.

### Example 91—Compound 34

Yield=0.069 g, 57%.

¹H NMR (400 MHz, CDCl₃) δ 7.74-7.52 (m, 4H), 7.39-7.21 (m, 4H), 7.02 (t, J=7.9 Hz, 1H), 6.77 (dd, J=8.1, 0.9 Hz, 1H), 6.38 (s, 1H), 5.94 (dd, J=7.8, 0.9 Hz, 1H), 4.27-4.16 (m, 2H), 3.36 (hept, J=6.9 Hz, 2H), 1.97-1.84 (m, 2H), 1.42 (s, 9H), 1.37 (d, J=7.4 Hz, 2H), 1.17 (d, J=6.9 Hz, 12H), 0.94 (t, J=7.4 Hz, 3H).

¹³C NMR (101 MHz, CDCl₃) δ 152.62, 151.41, 148.15, 140.31, 135.86, 134.94, 131.33, 129.11, 128.16, 127.26, 125.73, 123.74, 123.66, 102.26, 98.77, 44.70, 34.86, 32.10, 31.29, 28.15, 23.94, 20.11, 13.66.

### Example 92—Compound 35

Yield=0.039 g, 33%.

¹H NMR (400 MHz, CDCl₃) δ 7.70-7.54 (m, 4H), 7.21 (t, J=8.0 Hz, 1H), 7.04 (d, J=8.1 Hz, 1H), 6.89-6.78 (m, 2H), 6.58 (dt, J=7.2, 3.2 Hz, 1H), 4.29-4.19 (m, 2H), 1.86 (ddt, J=9.2, 7.7, 3.6 Hz, 2H), 1.41 (s, 9H), 1.35 (q, J=7.5 Hz, 2H), 0.92 (t, J=7.4 Hz, 3H).

¹³C NMR (101 MHz, CDCl₃) δ 153.02, 152.68, 136.04, 133.61, 133.31, 129.03, 127.64, 125.82, 123.08, 105.60, 103.04, 99.39, 99.16, 98.93, 44.77, 34.89, 31.96, 31.25, 20.00, 13.59.

### Example 93—Compound 36

Yield=0.054 g, 45%.

¹H NMR (400 MHz, CDCl₃) δ 8.24 (d, J=8.1 Hz, 1H), 7.90 (dd, J=7.6, 1.9 Hz, 1H), 7.75-7.43 (m, 10H), 7.30 (d, J=10.9 Hz, 2H), 7.14 (td, J=7.9, 2.7 Hz, 1H), 6.90 (ddd, J=16.9, 7.8, 2.7 Hz, 2H), 4.26 (t, J=7.6 Hz, 2H), 1.98-1.80 (m, 2H), 1.42 (s, 9H), 1.37 (q, J=8.0, 7.5 Hz, 2H), 0.94 (td, J=7.3, 2.7 Hz, 3H).

¹³C NMR (101 MHz, CDCl₃) δ 152.83, 152.08, 137.92, 137.17, 136.17, 134.75, 133.04, 129.07, 128.62, 128.34, 127.93, 126.08, 125.95, 125.80, 125.59, 123.44, 122.67, 117.56, 104.77, 100.80, 44.73, 34.88, 32.00, 31.28, 20.03, 13.64.

### Example 94—Compound 37

Yield=0.067 g, 56%.

¹H NMR (400 MHz, CDCl3) δ 7.86-7.65 (m, 6H), 7.59 (d, J=8.4 Hz, 2H), 7.50-7.42 (m, 3H), 7.40-7.22 (m, 5H), 6.98 (d, J=8.0 Hz, 1H), 4.26 (t, J=7.7 Hz, 2H), 1.88 (p, J=7.6 Hz, 2H), 1.42 (s, 9H), 1.38-1.28 (m, 2H), 0.93 (t, J=7.4 Hz, 3H).

¹³C NMR (101 MHz, CDCl3) δ 153.13, 151.98, 139.94, 136.04, 135.09, 134.61, 129.41, 129.04, 129.01, 127.65, 126.73, 126.32, 125.89, 123.66, 123.58, 121.05, 112.82, 105.23, 101.59, 44.77, 34.91, 31.94, 31.26, 20.00, 13.61.

### Example 95—Compound 38

Yield=0.076 g, 63%.

¹H NMR (400 MHz, CDCl₃) δ 7.73-7.68 (m, 1H), 7.62-7.48 (m, 6H), 7.44-7.29 (m, 5H), 7.23-7.08 (m, 3H), 6.92 (dd, J=7.1, 1.8 Hz, 1H), 6.76 (s, 1H), 4.25-4.15 (m, 2H), 1.92-1.81 (m, 2H), 1.38 (s, 9H), 1.36-1.31 (m, 2H), 0.92 (t, J=7.3 Hz, 3H).

¹³C NMR (101 MHz, CDCl₃) δ 152.68, 151.92, 139.35, 139.22, 136.32, 136.26, 133.65, 133.55, 131.13, 129.32, 128.99, 128.72, 127.97, 127.82, 127.28, 125.67, 123.28, 122.22, 120.72, 105.05, 101.17, 44.67, 34.83, 31.99, 31.25, 20.03, 13.62.

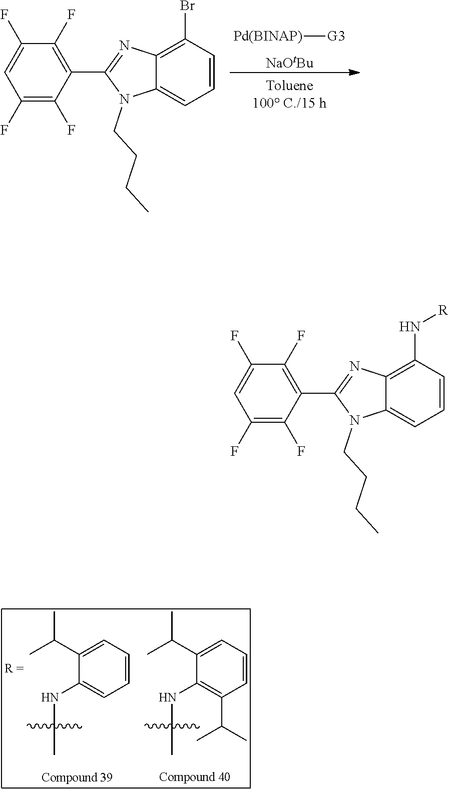

Buckwald-Hartwig Coupling for 4-bromo-1-butyl-2-(2,3,5,6-tetrafluorophenyl)-1H-benzo[d]imidazole:

The brominated compound and amines were provided for a Buchwald-Hartwig Cross Coupling reaction in a high throughput sequence beginning with CM3 manipulation.

Brominated starting material were provided and reacted with excess amine (2:1). All reactants/reagents were delivered in solution (Toluene) with the exception of sodium t-butoxide and the catalyst (weighed as solids). Reaction were diluted with additional reaction solvent to −10 mL before overnight reaction. The following day reaction conversion was checked via UPLC. After 16 hours at 95° C. conversion was high enough to proceed with purification.

Purification consisted of three phases: liquid/liquid extraction, filtration through a plug, and Supercritical Fluid Chromatography (SFC). After removal from the glove box, 5 mL of chloroform and 5 mL of saturated aqueous sodium chloride were added to the reaction vial. The vial was capped, shaken, quickly vented, and then poured off into a 25 mL Biotage ISOLUTE® Phase separator column. An additional 5 mL of chloroform was added and the organic phase was collected after gravity filtration. The collected material was poured into a GL Sciences 20 mL InertSep PS-SL filter and gravity filtered again. One wash of 5 mL chloroform was similarly used to rinse the phase separation column, then InertSep filter. A final rinse of the silica pad was performed with 5 mL ethyl acetate and the collected samples were concentrated over 10 hours at 80° C. under vacuum on a Savant SpeedVac, which ramped at 5 Torr/min. The solids were delivered back to T. Paine for purification on the SFC.

Preparative SFC was used to purify using a 1-AA 130 Å 5 μm OBD 30×150 mm column using CO2 as mobile phase A and 75% acetonitrile: 25% isopropanol as mobile phase B. The gradient used was 5% B to 50% B over 10 minutes with a total flow rate of 100 mL/min. The collection make-up solvent used was ethyl acetate, the BPR pressure was 100 bar, oven temp was 40° C., the sample concentration was 50 mg/mL and injection volume was 960 μL. The desired compounds were collected by mass spectrometry.

### Example 96—Compound 39

Yield=0.013 g, 11%.

¹H NMR (400 MHz, CDCl₃) δ 7.49 (dd, J=7.7, 1.7 Hz, 1H), 7.38 (ddd, J=8.8, 6.2, 2.7 Hz, 1H), 7.32 (ddd, J=9.5, 7.3, 2.2 Hz, 1H), 7.26-7.15 (m, 3H), 6.88 (dd, J=8.1, 0.9 Hz, 1H), 6.67 (dd, J=7.9, 0.9 Hz, 1H), 6.64 (s, 1H), 4.08 (t, J=7.4 Hz, 2H), 3.36 (p, J=6.9 Hz, 1H), 1.87-1.71 (m, 2H), 1.26 (d, J=6.9, 6+2H), 0.88 (t, J=7.3 Hz, 3H).

¹³C NMR (101 MHz, CDCl₃) δ 143.16, 138.66, 138.22, 135.61, 133.18, 126.40, 126.31, 126.27, 124.78, 124.76, 124.50, 108.34, 108.12, 103.59, 99.99, 99.93, 44.71, 31.51, 27.70, 23.39, 19.77, 13.47.

### Example 97—Compound 40

Yield=0.019 g, 17%.

¹H NMR (400 MHz, CDCl₃) δ 7.39-7.23 (m, 5H), 7.12-7.04 (m, 1H), 6.80 (dd, J=8.0, 3.2 Hz, 1H), 6.34 (s, 1H), 5.98 (dd, J=7.8, 3.3 Hz, 1H), 4.08 (t, J=7.4 Hz, 2H), 3.32 (hept, J=6.8 Hz, 2H), 1.87-1.74 (m, 2H), 1.30 (m, 2H), 1.18 (d, J=6.8 Hz, 12H), 0.90 (t, J=7.4 Hz, 3H).

¹³C NMR (101 MHz, CDCl₃) δ 148.06, 140.65, 135.48, 134.53, 132.07, 127.46, 124.94, 123.81, 102.54, 98.82, 44.75, 31.55, 28.56, 28.18, 24.19, 23.35, 19.85, 13.49.

### Example 98—PPR Screening

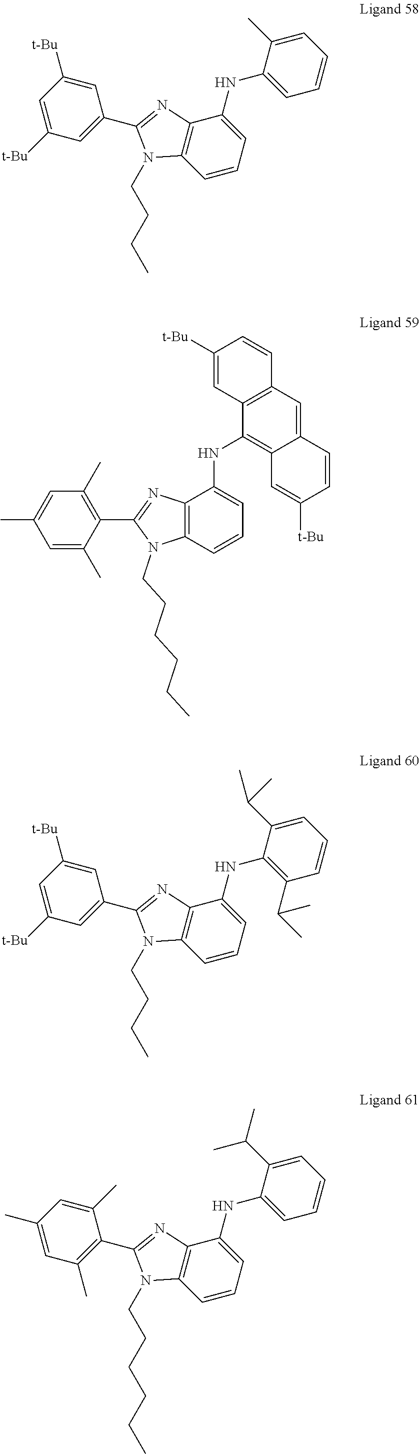 [SMILES: CCCCn1c(-c2cc(C(C)(C)C)cc(C(C)(C)C)c2)nc2c(Nc3ccccc3C)cccc21 ; CCCCCCn1c(-c2c(C)cc(C)cc2C)nc2c(Nc3c4cc(C(C)(C)C)ccc4cc4ccc(C(C)(C)C)cc34)cccc21 ; CCCCn1c(-c2cc(C(C)(C)C)cc(C(C)(C)C)c2)nc2c(Nc3c(C(C)C)cccc3C(C)C)cccc21 ; CCCCCCn1c(-c2c(C)cc(C)cc2C)nc2c(Nc3ccccc3C(C)C)cccc21]

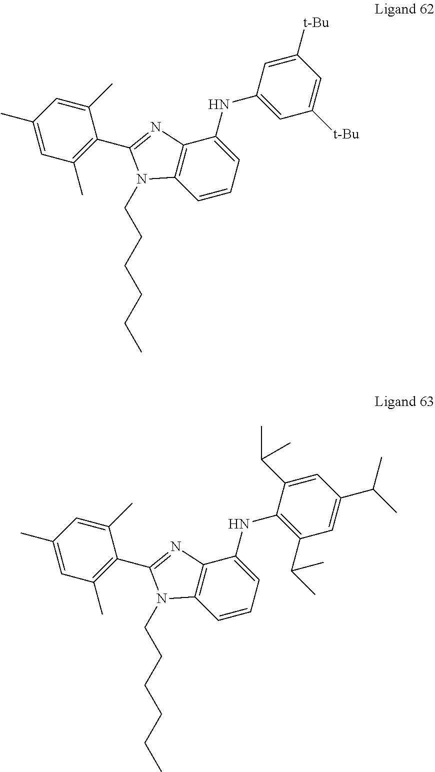 [SMILES: CCCCCCn1c(-c2c(C)cc(C)cc2C)nc2c(Nc3cc(C(C)(C)C)cc(C(C)(C)C)c3)cccc21 ; CCCCCCn1c(-c2c(C)cc(C)cc2C)nc2c(Nc3c(C(C)C)cc(C(C)C)cc3C(C)C)cccc21]

TABLE 1
PPR results for mesityl or 3,5-di-tert-butylphenyl-substituted amino-
benzimidazole ligands. Results are an average of two runs.

| Ligand-# | | | Mw | Quench | | Yield | Mol % |
|---|---|---|---|---|---|---|---|
| (L-#) | Metal | L:M | (g/mol) | (sec) | PDI | (mg) | Octene |
| L-58 | Hf | 1 | 188,600 | 52 | 6.4 | 210 | 10.3 |
| L-58 | Hf | 2 | 271,400 | 78 | 5.0 | 150 | 7.0 |
| L-58 | Zr | 1 | 329,800 | 84 | 26.8 | 160 | 4.6 |
| L-58 | Zr | 2 | 419,900 | 126 | 29.3 | 130 | 4.2 |
| L-59 | Hf | 1 | 158,000 | 28 | 16.0 | 330 | 14.0 |
| L-59 | Hf | 2 | 337,300 | 46 | 16.2 | 190 | 7.6 |
| L-59 | Zr | 1 | 239,100 | 50 | 45.7 | 170 | 6.4 |
| L-59 | Zr | 2 | 308,000 | 95 | 59.1 | 140 | 4.8 |
| L-60 | Hf | 1 | 74,130 | 21 | 14.7 | 350 | 12.8 |
| L-60 | Hf | 2 | 85,050 | 29 | 11.6 | 290 | 12.3 |
| L-60 | Zr | 1 | 156,100 | 43 | 18.2 | 190 | 5.8 |
| L-60 | Zr | 2 | 150,500 | 45 | 17.9 | 200 | 5.4 |
| L-61 | Hf | 1 | 147,900 | 32 | 8.4 | 270 | 11.8 |
| L-61 | Hf | 2 | 142,100 | 30 | 11.0 | 290 | 11.1 |
| L-61 | Zr | 1 | 366,400 | 64 | 27.7 | 150 | 4.8 |
| L-61 | Zr | 2 | 311,000 | 41 | 24.4 | 160 | 2.8 |
| L-62 | Hf | 1 | 199,000 | 46 | 7.0 | 180 | 7.1 |
| L-62 | Hf | 2 | 146,900 | 31 | 12.6 | 200 | 5.3 |
| L-62 | Zr | 1 | 201,200 | 91 | 11.3 | 140 | 3.2 |
| L-62 | Zr | 2 | 43,540 | 27 | 5.2 | 140 | 0.9 |
| L-63 | Hf | 1 | 99,420 | 29 | 17.9 | 320 | 13.4 |
| L-63 | Hf | 2 | 135,400 | 27 | 19.4 | 310 | 10.3 |
| L-63 | Zr | 1 | 377,900 | 57 | 30.5 | 130 | 3.3 |
| L-63 | Zr | 2 | 279,000 | 60 | 49.8 | 150 | 2.8 |

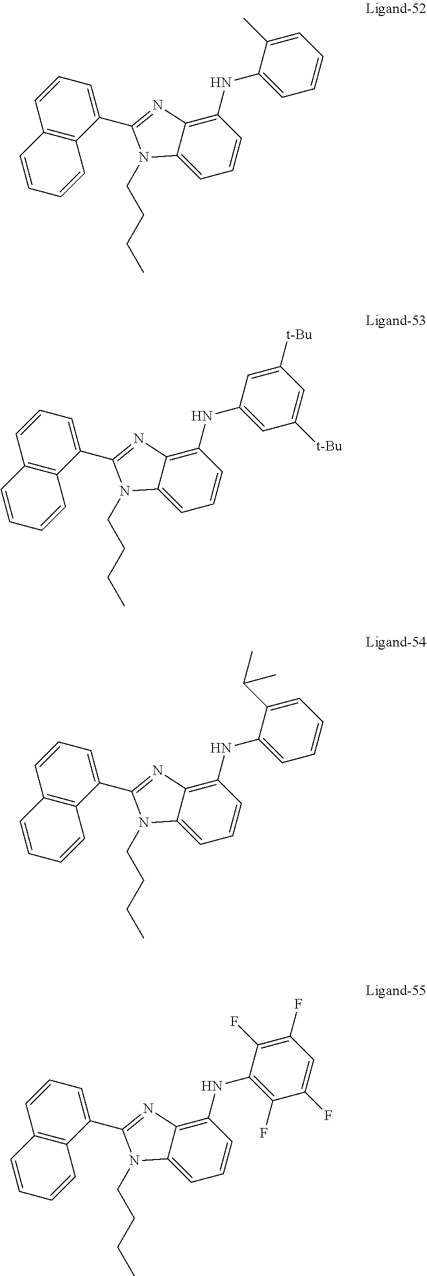 [SMILES: CCCCn1c(-c2cccc3ccccc23)nc2c(Nc3ccccc3C)cccc21 ; CCCCn1c(-c2cccc3ccccc23)nc2c(Nc3cc(C(C)(C)C)cc(C(C)(C)C)c3)cccc21 ; CCCCn1c(-c2cccc3ccccc23)nc2c(Nc3ccccc3C(C)C)cccc21 ; CCCCn1c(-c2cccc3ccccc23)nc2c(Nc3c(F)c(F)cc(F)c3F)cccc21]

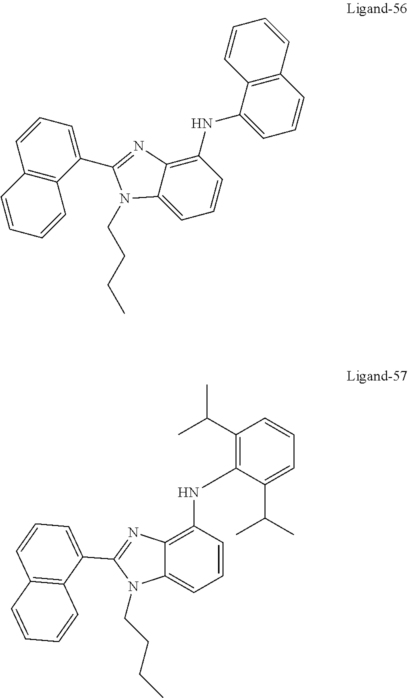 [SMILES: CCCCn1c(-c2cccc3ccccc23)nc2c(Nc3cccc4ccccc34)cccc21 ; CCCCn1c(-c2cccc3ccccc23)nc2c(Nc3c(C(C)C)cccc3C(C)C)cccc21]

TABLE 2
PPR results for naphthyl-substituted amino-benzimidazole ligands.

| Ligand-# | | | Mw | Quench | | Yield | Mol % |
|---|---|---|---|---|---|---|---|
| (L-#) | Metal | L:M | (g/mol) | (sec) | PDI | (mg) | Octene |
| L-52 | Hf | 1 | 157,400 | 40 | 12.6 | 280 | 9.9 |
| L-52 | Hf | 2 | 273,600 | 60 | 7.9 | 170 | 5.6 |
| L-52 | Zr | 1 | 248,000 | 64 | 22.5 | 170 | 4.0 |
| L-52 | Zr | 2 | 586,000 | 30 | 12.0 | 160 | −0.2 |
| L-53 | Hf | 1 | 197,400 | 52 | 5.0 | 140 | 4.7 |
| L-53 | Hf | 2 | 99,400 | 72 | 5.1 | 130 | 1.1 |
| L-53 | Zr | 1 | 217,400 | 78 | 10.7 | 140 | 1.4 |
| L-53 | Zr | 2 | 340,100 | 33 | 17.6 | 170 | 0.3 |
| L-54 | Hf | 1 | 160,900 | 46 | 4.1 | 160 | 8.1 |
| L-54 | Hf | 2 | 321,100 | 104 | 6.0 | 120 | 5.0 |
| L-54 | Zr | 1 | 280,400 | 81 | 20.7 | 170 | 4.0 |
| L-54 | Zr | 2 | 404,400 | 45 | 14.1 | 170 | 0.6 |
| L-55 | Hf | 1 | 176,900 | 123 | 3.7 | 120 | 0.5 |
| L-55 | Hf | 2 | 168,800 | 90 | 4.5 | 140 | 0.2 |
| L-55 | Zr | 1 | 252,600 | 59 | 8.2 | 140 | 0.9 |
| L-55 | Zr | 2 | 202,300 | 31 | 8.8 | 160 | 0.2 |
| L-56 | Hf | 1 | 243,800 | 36 | 13.1 | 220 | 8.8 |
| L-56 | Hf | 2 | 498,600 | 75 | 11.1 | 150 | 4.6 |
| L-56 | Zr | 1 | 320,300 | 72 | 28.1 | 180 | 4.4 |
| L-56 | Zr | 2 | 1,024,000 | 56 | 49.7 | 120 | 2.1 |
| L-57 | Hf | 1 | 78,680 | 24 | 11.0 | 320 | 10.1 |
| L-57 | Hf | 2 | 115,300 | 32 | 8.5 | 230 | 8.3 |
| L-57 | Zr | 1 | 154,300 | 48 | 15.6 | 180 | 4.0 |
| L-57 | Zr | 2 | 182,500 | 57 | 15.3 | 160 | 3.3 |

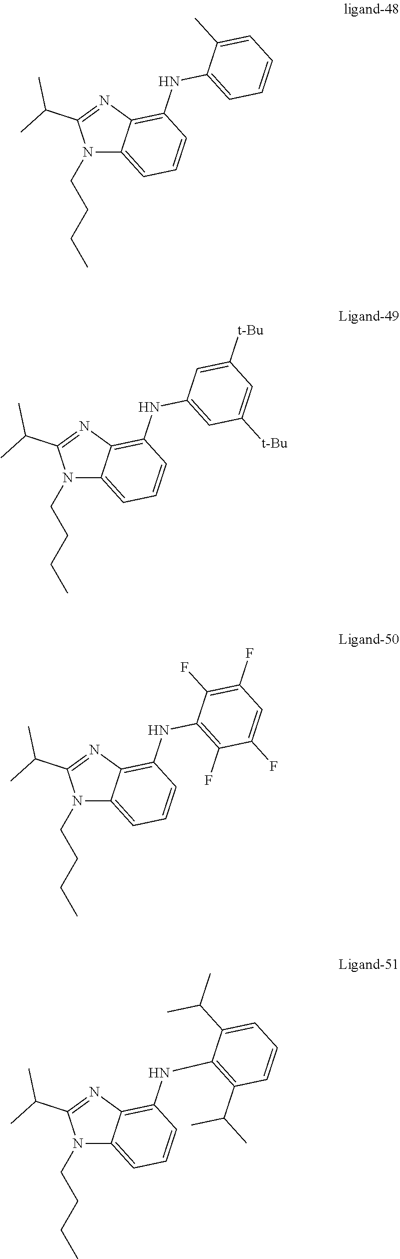 [SMILES: CCCCn1c(C(C)C)nc2c(Nc3ccccc3C)cccc21 ; CCCCn1c(C(C)C)nc2c(Nc3cc(C(C)(C)C)cc(C(C)(C)C)c3)cccc21 ; CCCCn1c(C(C)C)nc2c(Nc3c(F)c(F)cc(F)c3F)cccc21 ; CCCCn1c(C(C)C)nc2c(Nc3c(C(C)C)cccc3C(C)C)cccc21]

TABLE 3
PPR results for isopropyl-substituted amino-benzimidazole ligands.

| Ligand-# | | | Mw | Quench | | Yield | Mol % |
|---|---|---|---|---|---|---|---|
| (L-#) | Metal | L:M | (g/mol) | (sec) | PDI | (mg) | Octene |
| L-48 | Hf | 1 | 99,330 | 48 | 3.2 | 160 | 8.6 |
| L-48 | Hf | 2 | 195,100 | 239 | 4.6 | 100 | 6.6 |
| L-48 | Zr | 1 | 197,100 | 140 | 8.5 | 130 | 5.6 |
| L-48 | Zr | 2 | 250,900 | 680 | 15.9 | 90 | 3.5 |
| L-49 | Hf | 1 | 139,100 | 1,438 | 8.0 | 80 | 5.9 |
| L-49 | Hf | 2 | 68,920 | 1,788 | 11.0 | 70 | 1.6 |
| L-49 | Zr | 1 | 170,400 | 383 | 35.2 | 90 | 3.7 |
| L-49 | Zr | 2 | 8,991 | 82 | 3.4 | 90 | 0.9 |
| L-50 | Hf | 1 | 43,690 | 1,801 | 5.7 | 60 | 1.2 |
| L-50 | Hf | 2 | 17,870 | 243 | 2.9 | 70 | 0.8 |
| L-50 | Zr | 1 | 84,600 | 247 | 17.1 | 80 | 2.7 |
| L-50 | Zr | 2 | 9,264 | 54 | 2.7 | 100 | 1.1 |
| L-51 | Hf | 1 | 65,650 | 31 | 5.2 | 250 | 7.9 |
| L-51 | Hf | 2 | 129,600 | 60 | 4.7 | 150 | 5.4 |
| L-51 | Zr | 1 | 384,000 | 604 | 29.8 | 80 | 3.0 |
| L-51 | Zr | 2 | 364,800 | 893 | 18.9 | 80 | 3.0 |

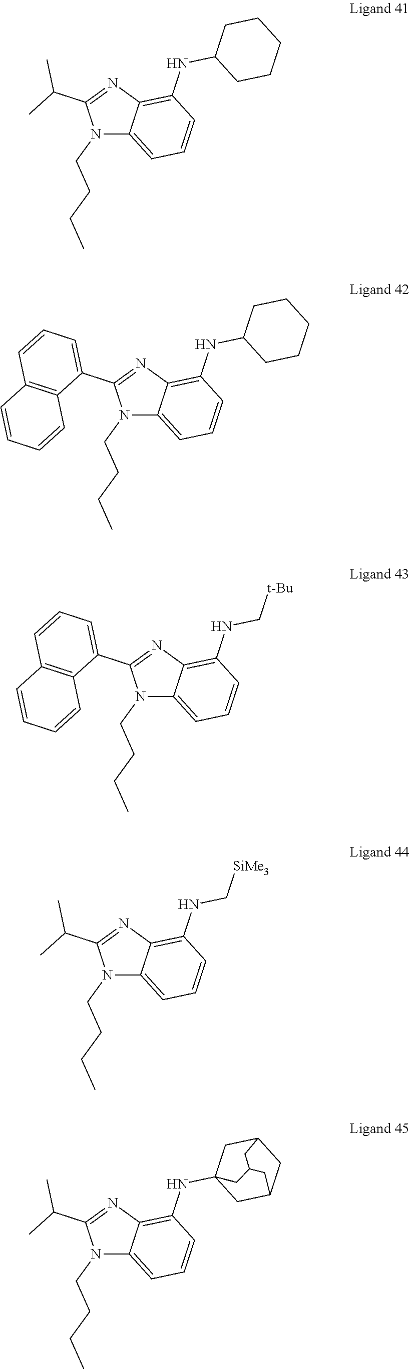 [SMILES: CCCCn1c(C(C)C)nc2c(NC3CCCCC3)cccc21 ; CCCCn1c(-c2cccc3ccccc23)nc2c(NC3CCCCC3)cccc21 ; CCCCn1c(-c2cccc3ccccc23)nc2c(NCC(C)(C)C)cccc21 ; CCCCn1c(C(C)C)nc2c(NC[Si](C)(C)C)cccc21 ; CCCCn1c(C(C)C)nc2c(NC34CC5CC(CC(C5)C3)C4)cccc21]

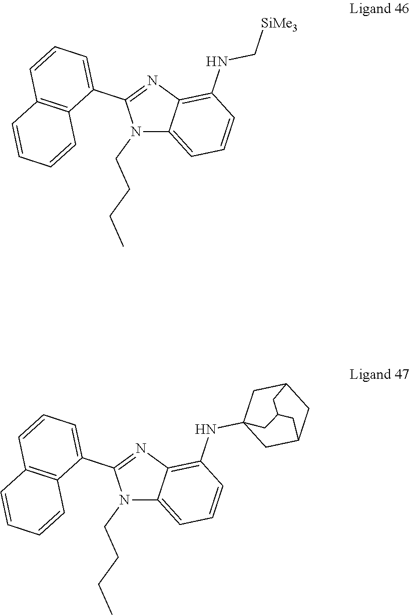 [SMILES: CCCCn1c(-c2cccc3ccccc23)nc2c(NC[Si](C)(C)C)cccc21 ; CCCCn1c(-c2cccc3ccccc23)nc2c(NC34CC5CC(CC(C5)C3)C4)cccc21]

TABLE 4
PPR results for alkyl-substituted amino-benzimidazole ligands.

| Ligand # | | | Mw | Quench | | Yield | Mol % |
|---|---|---|---|---|---|---|---|
| (L-#) | Metal | L:M | (g/mol) | (sec) | PDI | (mg) | Octene |
| L-41 | Hf | 1 | | 1,801 | | 10 | |
| L-41 | Hf | 2 | | 1,800 | | 10 | |
| L-41 | Zr | 1 | 504,200 | 1,802 | 76.9 | 60 | 2.5 |
| L-41 | Zr | 2 | 579,500 | 1,800 | 83.4 | 40 | 3.9 |
| L-42 | Hf | 1 | 235,000 | 1,800 | 16.4 | 40 | 2.3 |
| L-42 | Hf | 2 | 155,900 | 1,801 | 31.6 | 30 | 2.5 |
| L-42 | Zr | 1 | 520,500 | 998 | 66.6 | 70 | 2.3 |
| L-42 | Zr | 2 | 207,700 | 843 | 77.7 | 70 | 0.9 |
| L-43 | Hf | 1 | 367,200 | 834 | 15.3 | 70 | 3.0 |
| L-43 | Hf | 2 | | 1,801 | | 20 | |
| L-43 | Zr | 1 | 592,000 | 655 | 50.5 | 80 | 2.6 |
| L-43 | Zr | 2 | 366,900 | 1,801 | 25.8 | 50 | 0.8 |
| L-44 | Hf | 1 | 203,100 | 876 | 8.4 | 80 | 5.1 |
| L-44 | Hf | 2 | 275,400 | 1,801 | 18.1 | 40 | 4.4 |
| L-44 | Zr | 1 | 606,100 | 1,802 | 93.5 | 60 | 2.6 |
| L-44 | Zr | 2 | | 1,802 | | 20 | |
| L-45 | Hf | 1 | | 1,801 | | 0 | |
| L-45 | Hf | 2 | | 1,800 | | 0 | |
| L-45 | Zr | 1 | 462,100 | 1,802 | 83.9 | 50 | 3.0 |
| L-45 | Zr | 2 | 311,100 | 1,800 | 83.1 | 20 | 5.8 |
| L-46 | Hf | 1 | 433,300 | 147 | 11.4 | 130 | 3.5 |
| L-46 | Hf | 2 | 482,700 | 660 | 19.4 | 80 | 2.2 |
| L-46 | Zr | 1 | 454,600 | 314 | 44.5 | 80 | 1.6 |
| L-46 | Zr | 2 | 268,200 | 514 | 23.2 | 80 | 0.5 |
| L-47 | Hf | 1 | | 1,802 | | 10 | |
| L-47 | Hf | 2 | | 1,801 | | 0 | |
| L-47 | Zr | 1 | 491,700 | 1,802 | 97.8 | 40 | 3.0 |
| L-47 | Zr | 2 | | 1,801 | | 10 | |

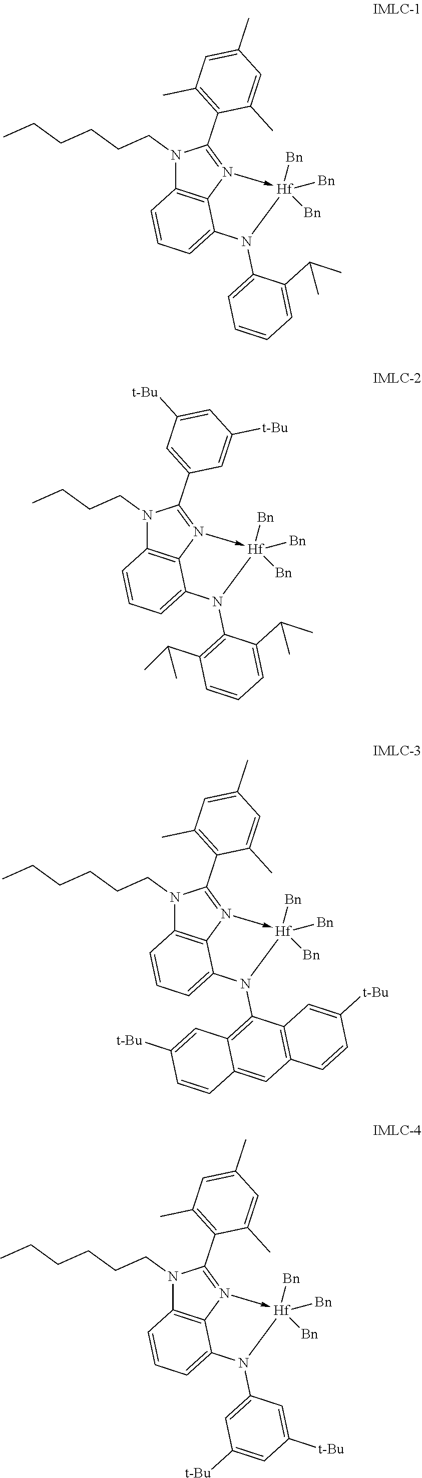

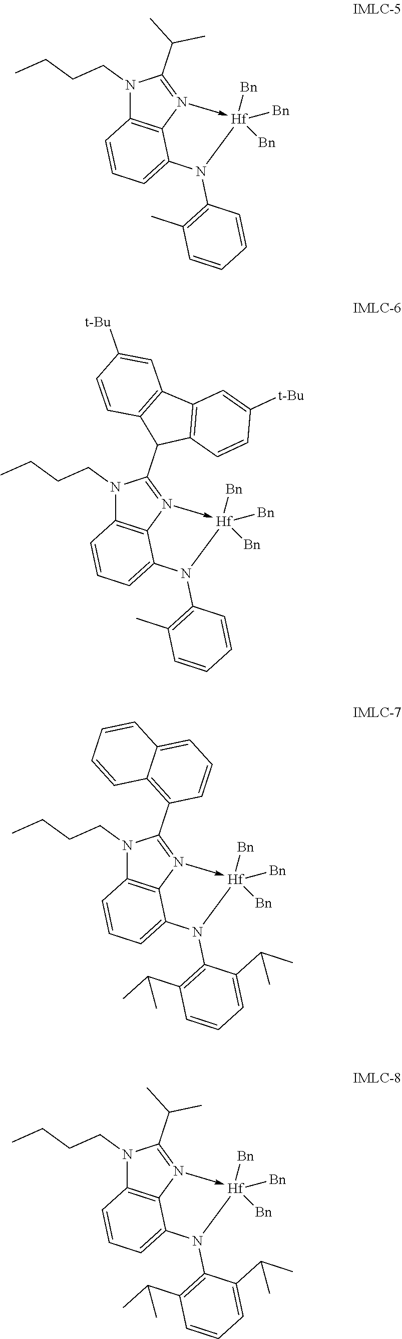

TABLE 5
Batch reactor results for aryl-substituted amino-benzimidazole ligands

| | Temp | Efficiency | Yield | Cat. Loading | Mw | | Tₘ | Mol % |
|---|---|---|---|---|---|---|---|---|
| IMLC# | (° C.) | (g poly/g metal) | (g) | (μmol) | (g/mol) | PDI | (° C.) | Octene |
| IMLC-1 | 120 | 142,865 | 5.1 | 0.20 | 513,312 | 3.4 | 96.8 | 3.8 |
| IMLC-1 | 150 | 248,380 | 13.3 | 0.30 | 411,198 | 2.2 | 95.5 | 4.2 |
| IMLC-2 | 120 | 801,165 | 14.3 | 0.10 | 584,955 | 7.1 | 96.3 | 4.1 |
| IMLC-2 | 150 | 476,217 | 15.3 | 0.18 | 275,763 | 10.6 | 92.3 | 4.6 |
| IMLC-3 | 120 | 243,711 | 8.7 | 0.20 | 803,042 | 3.6 | 97.7 | 3.6 |
| IMLC-3 | 150 | 360,164 | 22.5 | 0.35 | 523,616 | 3.1 | 91.0 | 4.7 |
| IMLC-4 | 120 | 114,852 | 4.1 | 0.20 | 303,351 | 5.1 | 122.6 | 3.4 |
| IMLC-4 | 150 | 106,449 | 7.6 | 0.40 | 395,657 | 6.6 | 88.8 | 4.6 |
| IMLC-5 | 120 | 28,013 | 0.5 | 0.10 | 94,049 | 19.2 | — | — |
| IMLC-5 | 150 | 63,496 | 3.4 | 0.30 | 99,551 | 8.3 | 74.7 | 7.7 |
| IMLC-6 | 120 | 78,436 | 1.4 | 0.10 | 212,135 | 4.6 | 90.7 | 6.4 |
| IMLC-6 | 150 | 239,789 | 10.7 | 0.25 | 218,885 | 3.0 | 80.1 | 6.5 |
| IMLC-7 | 120 | 442,602 | 7.9 | 0.10 | 330,649 | 3.7 | 101.0 | 3.4 |
| IMLC-7 | 150 | 532,243 | 9.5 | 0.10 | 214,753 | 2.8 | 99.3 | 3.3 |
| IMLC-8 | 120 | 459,409 | 20.5 | 0.25 | 563,759 | 5.6 | 91.6 | 4.8 |
| IMLC-8 | 150 | 329,430 | 14.7 | 0.25 | 145,600 | 2.6 | 93.0 | 5.0 |

Semi-batch reactor conditions at 120° C., ethylene-octene copolymerization data for a series of amino-benzimidazole catalysts: 46.3 g of ethylene, 302 g of 1-octene, 612 g of Isopar E, 1.2 eq. of RIBS-2 activator with respect to catalyst, 10 μmol of MMAO-3A, 290 psi reactor pressure. Semi-batch reactor conditions at 150° C., ethylene-octene copolymerization data for a series of amino-benzimidazole catalysts: 43 g of ethylene, 301 g of 1-octene, 548 g of Isopar E, 1.2 eq. of RIBS-2 activator with respect to catalyst, 10 μmol of MMAO-3A, 327 psi reactor pressure.

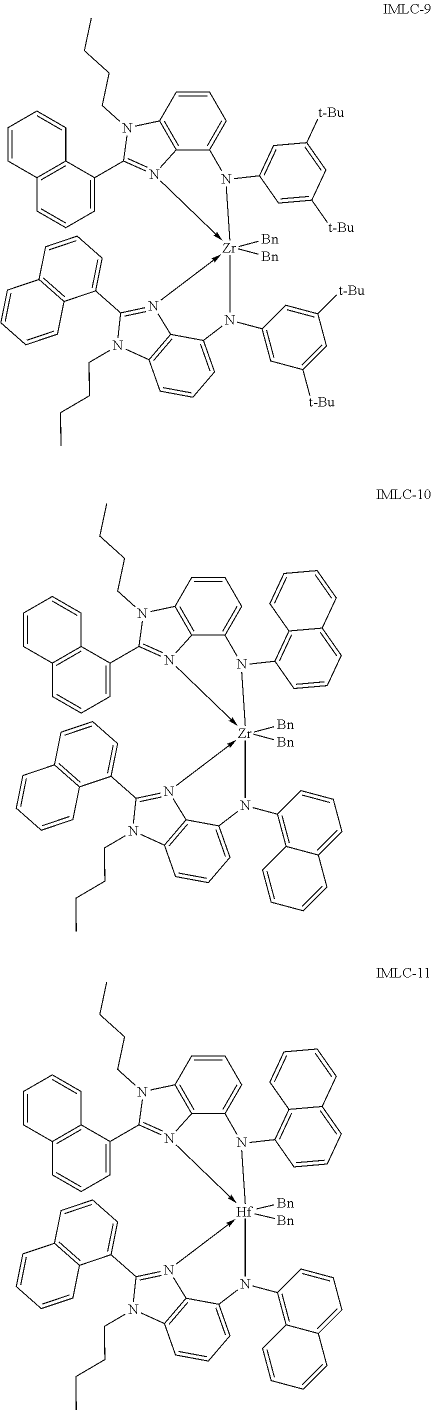

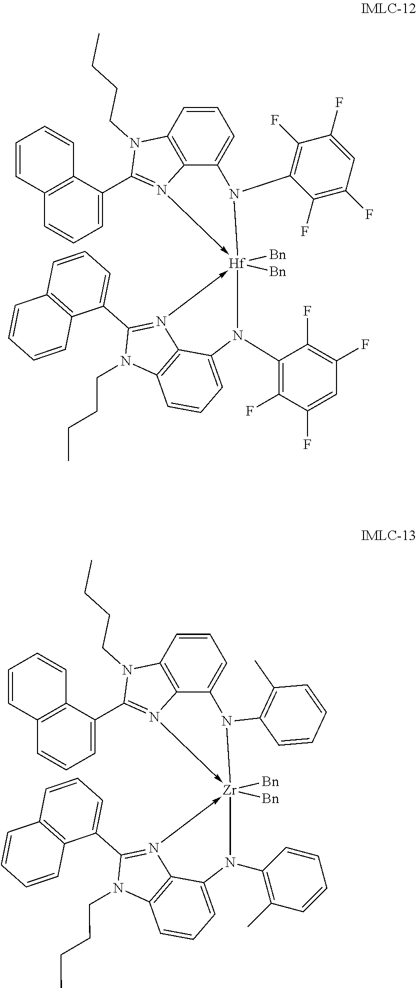

In Table 6, the polymerization and polymer results yielded from the metal-ligand complexes IMLC-9, IMLC-10, IMLC-11, IMLC-12, and IMLC-13 were tabulized. Comparative catalyst C1 (Comp. Cat C1) was run under the same conditions, and the polymerization results of Comp. Cat. C1 was recorded in Table 6.

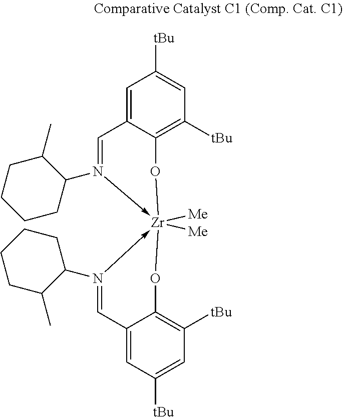

TABLE 6
Semi-batch Reactor data for a series of amino-benzimidazole catalysts

| | Temp | Efficiency | Yield | Cat. Loading | Mw | | Tₘ | Mol % |
|---|---|---|---|---|---|---|---|---|
| CS# | (° C.) | (gpoly/gM) | (g) | (μmol) | (g/mol) | PDI | (° C.) | Octene |
| IMLC-9 | 120 | 679,646 | 6.2 | 0.10 | 658,911 | 9.0 | 131.6 | 1.6 |
| IMLC-9 | 150 | 147,987 | 2.7 | 0.20 | 238,522 | 4.6 | 132.0 | 0.0 |
| IMLC-10 | 120 | 427,519 | 3.9 | 0.10 | 331,922 | 13.7 | 117.9 | 2.4 |
| IMLC-10 | 150 | 208,279 | 3.8 | 0.20 | 390,581 | 42.9 | 127.2 | 2.2 |
| IMLC-11 | 120 | 84,038 | 1.5 | 0.10 | 330,726 | 8.7 | 119.3 | 3.0 |
| IMLC-11 | 150 | 74,701 | 4.0 | 0.30 | 331,082 | 36.2 | 101.1 | 2.8 |
| IMLC-12 | 120 | 131,660 | 4.7 | 0.20 | 580,069 | 3.9 | 133.5 | 0.9 |
| IMLC-12 | 150 | 109,250 | 3.9 | 0.20 | 179,439 | 2.7 | 133.5 | 0.2 |
| IMLC-13 | 120 | 482,329 | 4.4 | 0.10 | 676,141 | 11.5 | 132.1 | 3.5 |
| IMLC-13 | 150 | 745,418 | 6.8 | 0.10 | 458,868 | 4.2 | 135.7 | 0.3 |
| Comp. Cat C1 | 120 | 335,124 | 21.4 | 0.70 | 617,083 | 2.6 | 123.4 | 0.8 |
| Comp. Cat C1 | 150 | 76,685 | 15.6 | 2.23 | 263,203 | 2.9 | 121.0 | 0.7 |

Semi-batch reactor conditions at 120° C., ethylene-octene copolymerization data: 46.3 g of ethylene, 302 g of 1-octene, 612 g of Isopar E, 1.2 eq. of RIBS-2 activator with respect to catalyst, 10 μmol of MMAO-3A, 290 psi reactor pressure. Semi-batch reactor conditions at 150° C. ethylene-octene copolymerization data for a series of amino-benzimidazole. 43 g of ethylene, 301 g of 1-octene, 548 g of Isopar E, 1.2 eq. of RIBS-2 activator with respect to catalyst, 10 of μmol MMAO-3A, 327 psi reactor pressure.

### Example 100—Chain Shuttling Ability

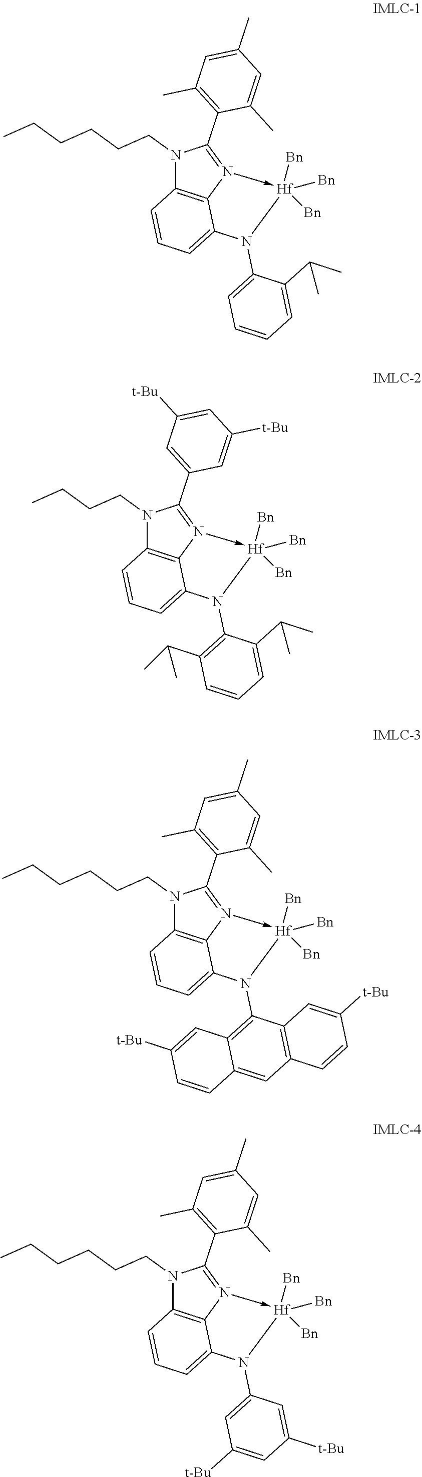

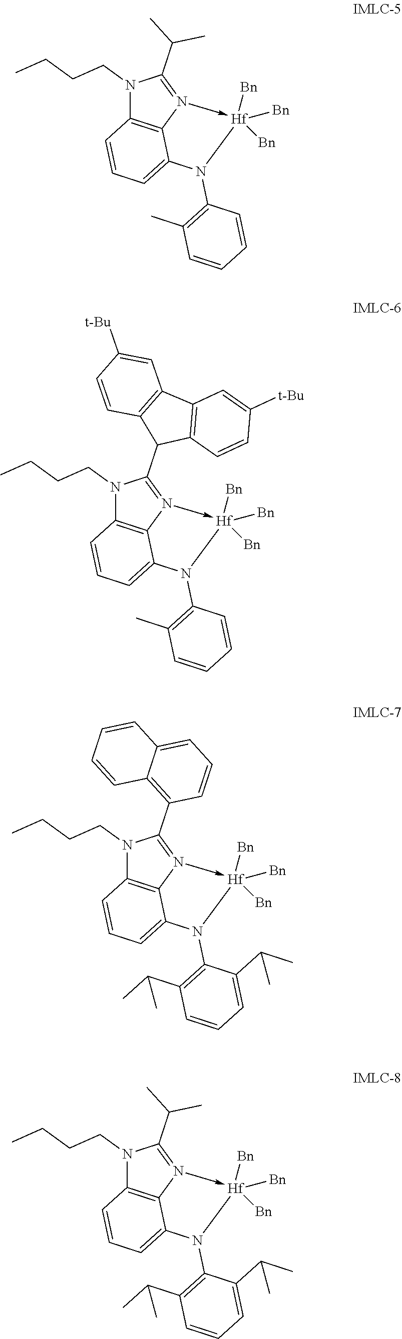

TABLE 7
Ethylene-octene copolymerization data under chain-transfer
conditions for a series of amino-benzimidazole catalysts

| | DEZ | Efficiency | Yield | Cat. Loading | Mw | | Tₘ | Mol % |
|---|---|---|---|---|---|---|---|---|
| IMLC-# | (μmole) | (g poly/g metal) | (g) | (μmole) | (g/mol) | PDI | (° C.) | Octene |
| IMLC-1 | 0 | 214,765 | 11.5 | 0.30 | 596,194 | 2.2 | 101.8 | 3.1 |
| IMLC-1 | 50 | 42,019 | 3.0 | 0.40 | 50,832 | 2.3 | 111.2 | 2.7 |
| IMLC-1 | 200 | 23,811 | 3.4 | 0.80 | 21,859 | 2.4 | 112.3 | 2.8 |
| IMLC-2 | 0 | 607,877 | 21.7 | 0.20 | 462,253 | 2.4 | 93.7 | 4.2 |
| IMLC-2 | 50 | 634,107 | 24.9 | 0.22 | 224,741 | 2.0 | 100.8 | 3.5 |
| IMLC-2 | 200 | 371,636 | 19.9 | 0.30 | 71,658 | 1.9 | 104.3 | 3.6 |
| IMLC-3 | 0 | 176,480 | 12.6 | 0.40 | 825,247 | 3.0 | 99.7 | 3.5 |
| IMLC-3 | 50 | 186,752 | 20.0 | 0.60 | 192,423 | 2.3 | 105.8 | 3.1 |
| IMLC-3 | 200 | 138,663 | 19.8 | 0.80 | 54,718 | 1.9 | 106.9 | 2.7 |
| IMLC-4 | 0 | 60,828 | 7.6 | 0.70 | 491,592 | 3.8 | 100.5 | 2.6 |
| IMLC-4 | 50 | 9,604 | 2.4 | 1.40 | 45,702 | 2.4 | 119.9 | 1.9 |
| IMLC-4 | 200 | 9,960 | 3.2 | 1.80 | 18,269 | 2.4 | 119.9 | 2.0 |
| IMLC-5 | 0 | 18,675 | 1.0 | 0.30 | 197,074 | 27.6 | 82.5 | 12.3 |
| IMLC-6 | 0 | 226,903 | 8.1 | 0.20 | 371,046 | 2.5 | 94.6 | 4.2 |
| IMLC-6 | 50 | 92,442 | 6.6 | 0.40 | 139,152 | 3.3 | 99.9 | 3.8 |
| IMLC-6 | 200 | 44,020 | 3.3 | 0.42 | 37,108 | 4.9 | 101.2 | 3.7 |
| IMLC-7 | 0 | 653,631 | 17.5 | 0.15 | 361,893 | 2.3 | 103.1 | 2.6 |
| IMLC-7 | 50 | 156,872 | 5.6 | 0.2 | 45,606 | 2.3 | 111.7 | 3.0 |
| IMLC-7 | 200 | 329,150 | 23.5 | 0.4 | 36,003 | 2.0 | 109.3 | 2.4 |
| IMLC-8 | 0 | 235,307 | 16.8 | 0.4 | 404,576 | 2.2 | 96.6 | 4.0 |
| IMLC-8 | 50 | 176,294 | 23.6 | 0.75 | 144,234 | 2.0 | 92.8 | 5.1 |
| IMLC-8 | 200 | 177,489 | 39.6 | 1.25 | 86,671 | 1.6 | 98.1 | 4.7 |

Semi-batch reactor conditions at 120° C.: 11.3 g of ethylene, 57 g of 1-octene, 557 g of Isopar E, 1.2 eq. of RIBS-2 activator with respect to catalyst, 10 μmol of MMAO-3A, 138 psi reactor pressure.

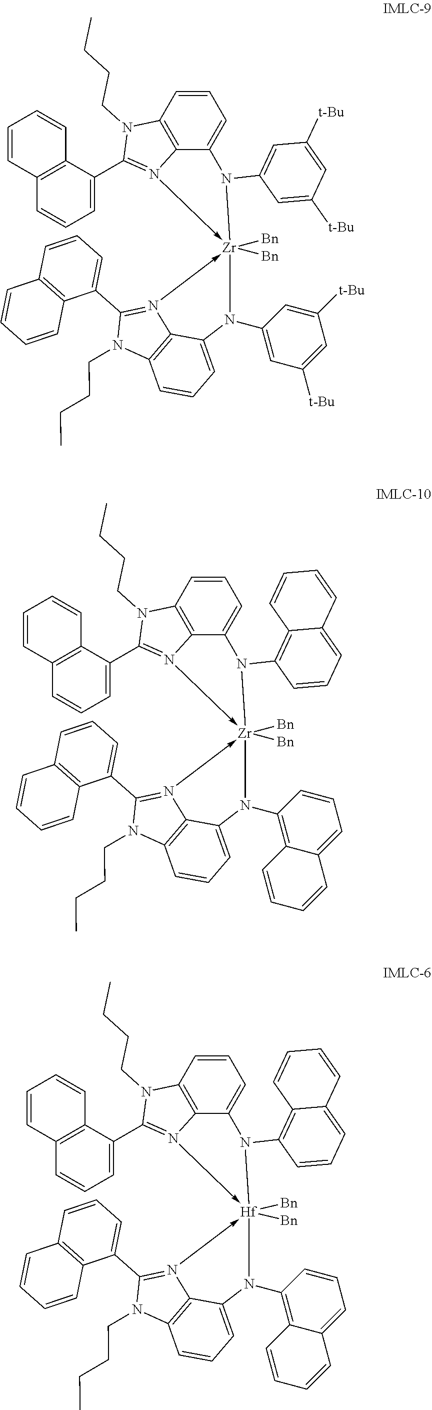

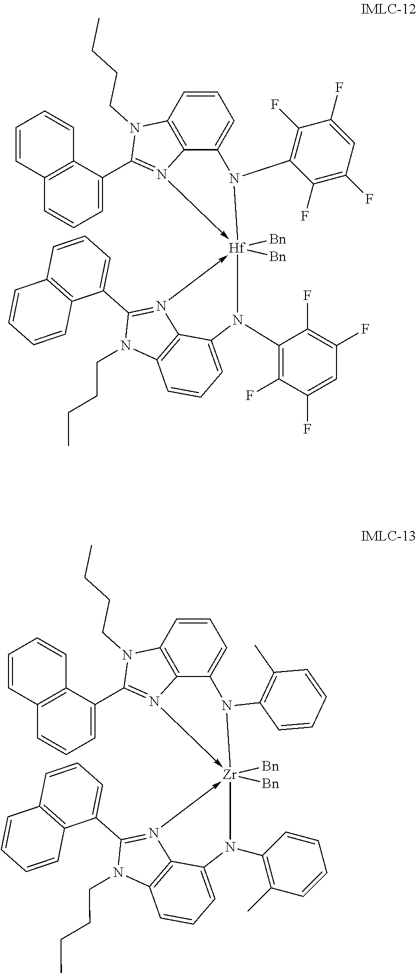

TABLE 8
Ethylene-octene copolymerization data under chain-transfer
conditions for a series of amino-benzimidazole catalysts

| | DEZ | Efficiency | Yield | Cat. Loading | Mw | | Tₘ | Mol % |
|---|---|---|---|---|---|---|---|---|
| IMLC-# | (μmole) | (g poly/g metal) | (g) | (μmol) | (g/mol) | PDI | (° C.) | Octene |
| IMLC-9 | 0 | 197,316 | 3.6 | 0.20 | 534,934 | 5.6 | 132.7 | 0.0 |
| IMLC-9 | 50 | 172,956 | 7.1 | 0.45 | 238,667 | 5.2 | 133.0 | 0.0 |
| IMLC-9 | 200 | 269,353 | 8.6 | 0.35 | 145,827 | 7.1 | 135.3 | 0.0 |
| IMLC-10 | 0 | 98,658 | 0.9 | 0.10 | 405,697 | 12.0 | 119.5 | 1.8 |
| IMLC-10 | 50 | 93,177 | 1.7 | 0.20 | 132,605 | 9.5 | 121.1 | 1.6 |
| IMLC-10 | 200 | 105,235 | 2.4 | 0.25 | 105,440 | 28.9 | 121.9 | 3.0 |
| IMLC-11 | 0 | 95,243 | 3.4 | 0.20 | 625,247 | 9.0 | 113.2 | 2.1 |
| IMLC-11 | 50 | 79,836 | 5.7 | 0.40 | 137,592 | 3.6 | 114.9 | 2.0 |
| IMLC-11 | 200 | 36,417 | 2.6 | 0.40 | 61,515 | 9.1 | 120.4 | 2.0 |
| IMLC-12 | 0 | 74,701 | 2.0 | 0.15 | 396,722 | 2.8 | 138.4 | 0.3 |
| IMLC-12 | 50 | 11,205 | 0.4 | 0.20 | 128,761 | 4.0 | 132.4 | 0.0 |
| IMLC-12 | 200 | 46,688 | 2.5 | 0.30 | 43,857 | 3.6 | 130.8 | 0.2 |
| IMLC-13 | 0 | 274,051 | 2.5 | 0.10 | 848,594 | 6.1 | 135.1 | 0.6 |
| IMLC-13 | 50 | 263,089 | 2.4 | 0.10 | 605,184 | 5.4 | 136.6 | 0.0 |
| IMLC-13 | 200 | 230,203 | 2.1 | 0.10 | 384,313 | 6.4 | 138.8 | 0.0 |

Semi-batch reactor conditions at 120° C.: 11.3 g of ethylene, 57 g of 1-octene, 557 g of Isopar E, 1.2 eq. of RIBS-2 activator with respect to catalyst, 10 μmol of MMAO-3A, 138 psi reactor pressure.

TABLE 9
Chain-transfer constants (Ca), average PDI over the
three runs, and standard deviation of the PDIs.

| | PDI | PDI | |
|---|---|---|---|
| CS# | (Avg.) | (Std. Dev.) | Ca |
| IMLC-1 | 2.3 | 0.1 | 4.7 |
| IMLC-2 | 2.1 | 0.2 | 0.5 |
| IMLC-3 | 2.4 | 0.5 | 1.0 |
| IMLC-4 | 2.9 | 0.7 | 5.0 |
| IMLC-9 | 6.0 | 0.8 | 1.2 |
| IMLC-2 | 3.6 | 1.0 | 2.2 |
| IMLC-7 | 2.2 | 0.1 | 4.2 |
| IMLC-8 | 1.9 | 0.2 | 0.8 |
| IMLC-10 | 16.8 | 8.6 | 5.2 |
| IMLC-11 | 7.2 | 2.6 | 1.7 |
| IMLC-12 | 3.5 | 0.5 | 2.6 |
| IMLC-13 | 6.0 | 0.4 | 0.2 |

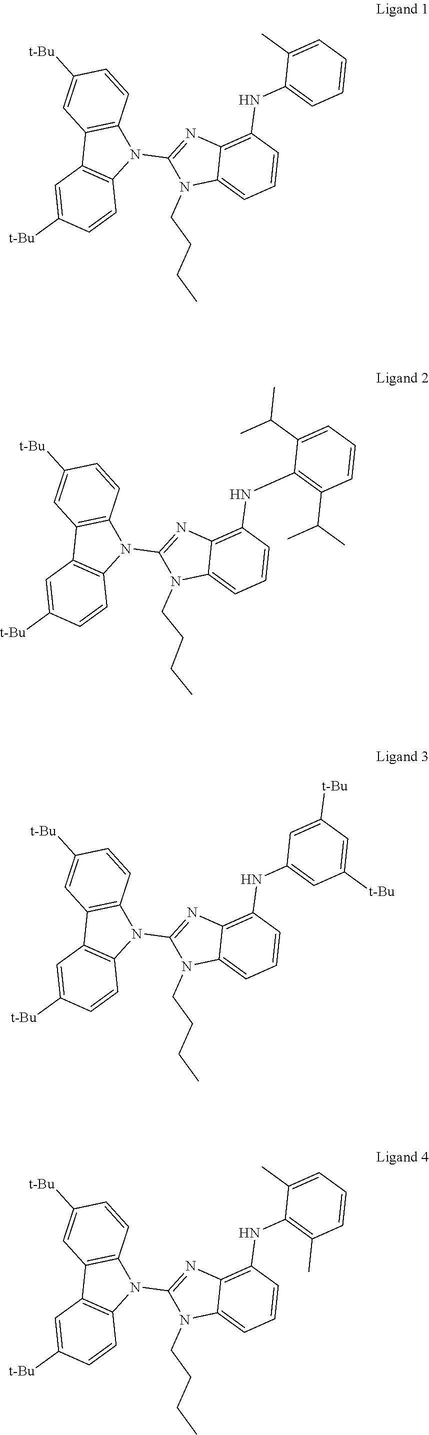 [SMILES: CCCCn1c(-n2c3ccc(C(C)(C)C)cc3c3cc(C(C)(C)C)ccc32)nc2c(Nc3ccccc3C)cccc21 ; CCCCn1c(-n2c3ccc(C(C)(C)C)cc3c3cc(C(C)(C)C)ccc32)nc2c(Nc3c(C(C)C)cccc3C(C)C)cccc21 ; CCCCn1c(-n2c3ccc(C(C)(C)C)cc3c3cc(C(C)(C)C)ccc32)nc2c(Nc3cc(C(C)(C)C)cc(C(C)(C)C)c3)cccc21 ; CCCCn1c(-n2c3ccc(C(C)(C)C)cc3c3cc(C(C)(C)C)ccc32)nc2c(Nc3c(C)cccc3C)cccc21]

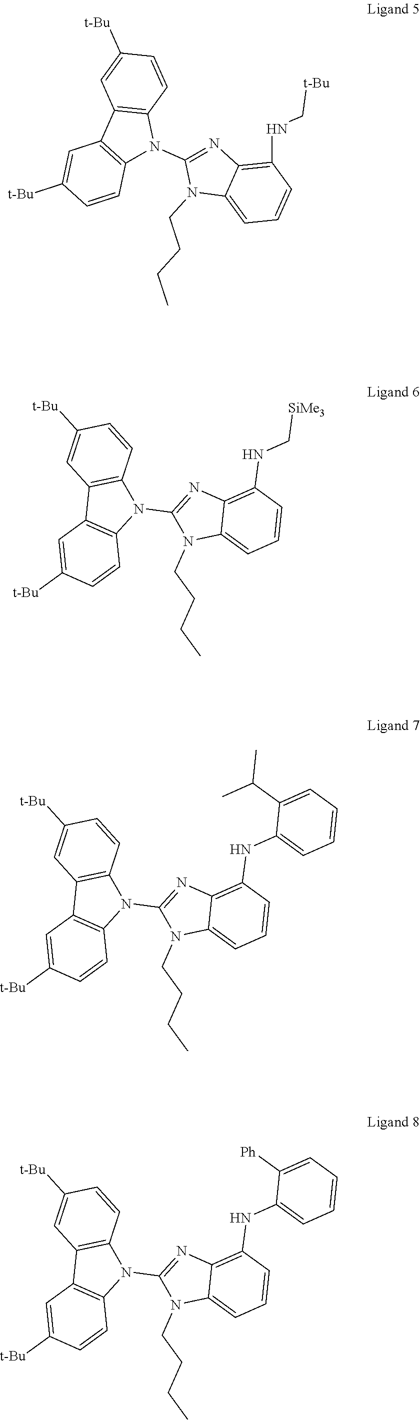 [SMILES: CCCCn1c(-n2c3ccc(C(C)(C)C)cc3c3cc(C(C)(C)C)ccc32)nc2c(NCC(C)(C)C)cccc21 ; CCCCn1c(-n2c3ccc(C(C)(C)C)cc3c3cc(C(C)(C)C)ccc32)nc2c(NC[Si](C)(C)C)cccc21 ; CCCCn1c(-n2c3ccc(C(C)(C)C)cc3c3cc(C(C)(C)C)ccc32)nc2c(Nc3ccccc3C(C)C)cccc21 ; CCCCn1c(-n2c3ccc(C(C)(C)C)cc3c3cc(C(C)(C)C)ccc32)nc2c(Nc3ccccc3-c3ccccc3)cccc21]

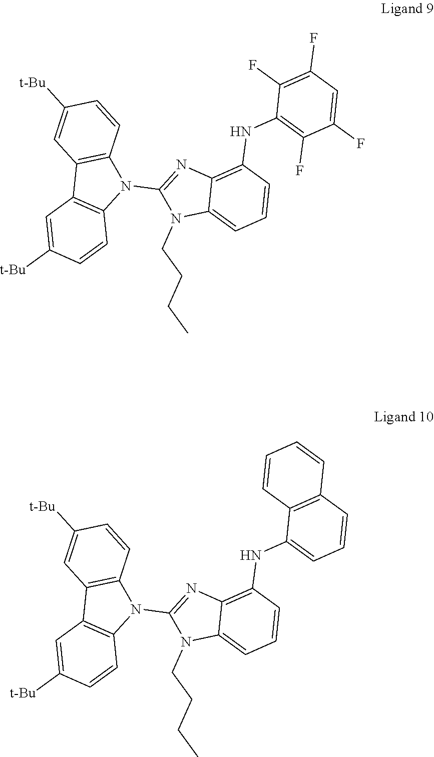 [SMILES: CCCCn1c(-n2c3ccc(C(C)(C)C)cc3c3cc(C(C)(C)C)ccc32)nc2c(Nc3c(F)c(F)cc(F)c3F)cccc21 ; CCCCn1c(-n2c3ccc(C(C)(C)C)cc3c3cc(C(C)(C)C)ccc32)nc2c(Nc3cccc4ccccc34)cccc21]

TABLE 10
PPR results for carbazole-based amino-benzimidazole catalysts.

| Ligand # | | L/M | Quench | Yield | Mw | | Mol % |
|---|---|---|---|---|---|---|---|
| (L-#) | Metal | ratio | (sec) | (g) | (g/mol) | PDI | Octene |
| L-1 | Hf(Bn)₄ | 1 | 54 | 0.17 | 221,200 | 4.7 | 6.9 |
| L-1 | Hf(Bn)₄ | 2 | 99 | 0.12 | 597,600 | 10.0 | 0.9 |
| L-1 | Zr(Bn)₄ | 1 | 60 | 0.17 | 194,100 | 18.5 | 6.0 |
| L-1 | Zr(Bn)₄ | 2 | 42 | 0.14 | 618,250 | 9.6 | 0.4 |
| L-2 | Hf(Bn)₄ | 1 | 31 | 0.22 | 99,265 | 3.5 | 8.8 |
| L-2 | Hf(Bn)₄ | 2 | 1,802 | 0.03 | 211,350 | 82.2 | −0.9 |
| L-2 | Zr(Bn)₄ | 1 | 52 | 0.15 | 160,300 | 35.1 | 7.2 |
| L-2 | Zr(Bn)₄ | 2 | 1,112 | 0.04 | 603,700 | 199.9 | 4.5 |
| L-3 | Hf(Bn)₄ | 1 | 116 | 0.13 | 235,750 | 21.2 | 4.2 |
| L-3 | Hf(Bn)₄ | 2 | 349 | 0.08 | 248,950 | 4.4 | −0.1 |
| L-3 | Zr(Bn)₄ | 1 | 78 | 0.15 | 135,350 | 29.2 | 3.5 |
| L-3 | Zr(Bn)₄ | 2 | 90 | 0.12 | 289,300 | 4.6 | −0.1 |
| L-4 | Hf(Bn)₄ | 1 | 35 | 0.19 | 128,200 | 4.0 | 8.2 |
| L-4 | Hf(Bn)₄ | 2 | 1,561 | 0.05 | 225,650 | 15.4 | 2.7 |
| L-4 | Zr(Bn)₄ | 1 | 106 | 0.14 | 322,550 | 18.4 | 4.3 |
| L-4 | Zr(Bn)₄ | 2 | 195 | 0.10 | 1,082,000 | 29.8 | 0.4 |
| L-5 | Hf(Bn)₄ | 1 | 1,801 | 0.06 | 121,000 | 4.9 | 1.1 |
| L-5 | Hf(Bn)₄ | 2 | 1,802 | 0.00 | | | |
| L-5 | Zr(Bn)₄ | 1 | 1,801 | 0.07 | 424,800 | 46.5 | 1.5 |
| L-5 | Zr(Bn)₄ | 2 | 1,802 | 0.03 | 252,225 | 33.2 | −1.8 |
| L-6 | Hf(Bn)₄ | 1 | 187 | 0.10 | 237,450 | 4.8 | 2.9 |
| L-6 | Hf(Bn)₄ | 2 | 1,802 | 0.02 | | | |
| L-6 | Zr(Bn)₄ | 1 | 196 | 0.09 | 211,200 | 20.0 | 1.8 |
| L-6 | Zr(Bn)₄ | 2 | 754 | 0.08 | 404,000 | 17.5 | 0.2 |
| L-7 | Hf(Bn)₄ | 1 | 59 | 0.16 | 209,850 | 2.7 | 7.8 |
| L-7 | Hf(Bn)₄ | 2 | 89 | 0.13 | 377,800 | 3.5 | −0.5 |
| L-7 | Zr(Bn)₄ | 1 | 59 | 0.17 | 240,100 | 6.4 | 6.7 |
| L-7 | Zr(Bn)₄ | 2 | 39 | 0.16 | 486,000 | 6.1 | −0.1 |
| L-8 | Hf(Bn)₄ | 1 | 97 | 0.12 | 158,400 | 2.4 | 3.5 |
| L-8 | Hf(Bn)₄ | 2 | 364 | 0.09 | 213,300 | 4.4 | 1.0 |
| L-8 | Zr(Bn)₄ | 1 | 592 | 0.09 | 276,600 | 38.4 | 6.3 |
| L-8 | Zr(Bn)₄ | 2 | 1,802 | 0.02 | 320,300 | 49.8 | 2.3 |
| L-9 | Hf(Bn)₄ | 1 | 1,341 | 0.07 | 405,200 | 14.3 | 1.8 |
| L-9 | Hf(Bn)₄ | 2 | 1,758 | 0.07 | 334,850 | 13.7 | 0.5 |
| L-9 | Zr(Bn)₄ | 1 | 131 | 0.11 | 216,100 | 21.0 | 4.2 |
| L-9 | Zr(Bn)₄ | 2 | 80 | 0.10 | 286,700 | 15.2 | 0.7 |
| L-10 | Hf(Bn)₄ | 1 | 878 | 0.07 | 1,356,000 | 20.1 | 0.2 |
| L-10 | Hf(Bn)₄ | 2 | 922 | 0.06 | 1,545,000 | 11.8 | 0.3 |
| L-10 | Zr(Bn)₄ | 1 | 690 | 0.06 | 1,532,000 | 82.9 | 0.9 |
| L-10 | Zr(Bn)₄ | 2 | 907 | 0.07 | 1,773,000 | 15.4 | 0.2 |

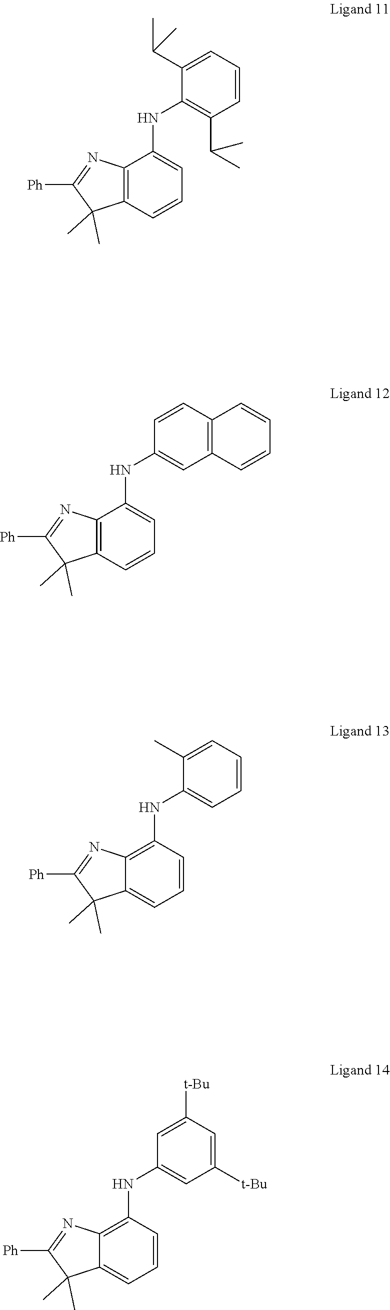 [SMILES: CC(C)c1cccc(C(C)C)c1Nc1cccc2c1N=C(c1ccccc1)C2(C)C ; CC1(C)C(c2ccccc2)=Nc2c(Nc3ccc4ccccc4c3)cccc21 ; Cc1ccccc1Nc1cccc2c1N=C(c1ccccc1)C2(C)C ; CC(C)(C)c1cc(Nc2cccc3c2N=C(c2ccccc2)C3(C)C)cc(C(C)(C)C)c1]

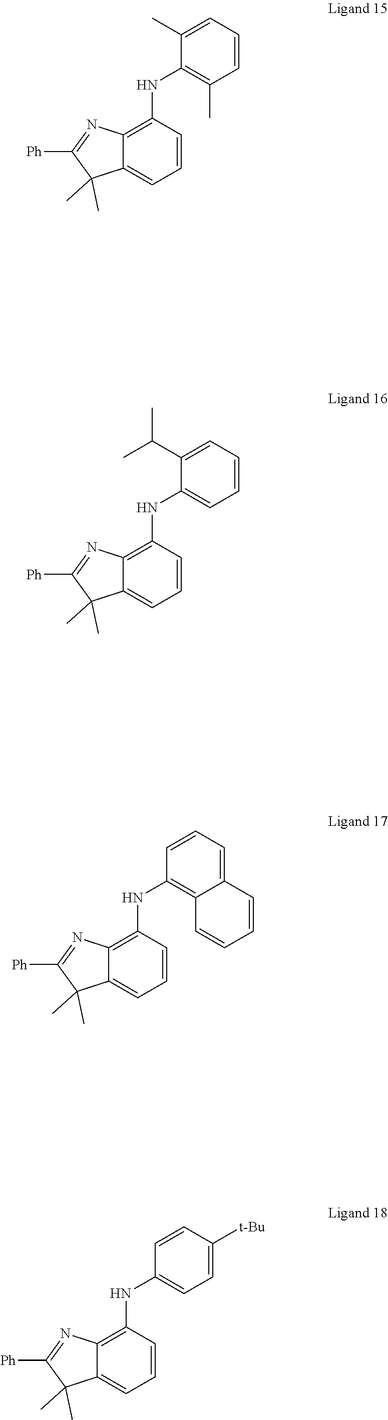 [SMILES: Cc1cccc(C)c1Nc1cccc2c1N=C(c1ccccc1)C2(C)C ; CC(C)c1ccccc1Nc1cccc2c1N=C(c1ccccc1)C2(C)C ; CC1(C)C(c2ccccc2)=Nc2c(Nc3cccc4ccccc34)cccc21 ; CC(C)(C)c1ccc(Nc2cccc3c2N=C(c2ccccc2)C3(C)C)cc1]

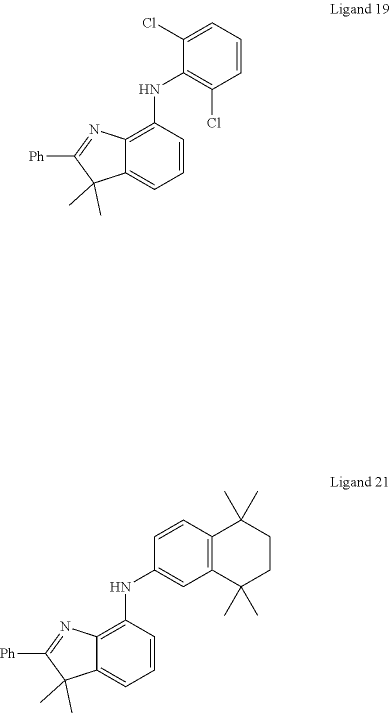 [SMILES: CC1(C)C(c2ccccc2)=Nc2c(Nc3c(Cl)cccc3Cl)cccc21 ; CC1(C)CCC(C)(C)c2cc(Nc3cccc4c3N=C(c3ccccc3)C4(C)C)ccc21]

TABLE 11
PPR results for gem-dimethyl based
amino-benzimidazole catalysts.

| Ligand# | | L/M | Quench | Yield | Mw | | Mol % |
|---|---|---|---|---|---|---|---|
| (L-#) | Metal | ratio | (sec) | (g) | (g/mol) | PDI | Octene |
| L-11 | Hf(Bn)₄ | 1 | 67 | 0.26 | 278,450 | 16.2 | 7.5 |
| L-11 | Hf(Bn)₄ | 2 | 539 | 0.11 | 465,700 | 24.9 | 2.6 |
| L-11 | Zr(Bn)₄ | 1 | 57 | 0.19 | 449,450 | 27.4 | 4.2 |
| L-11 | Zr(Bn)₄ | 2 | 182 | 0.12 | 621,150 | 19.1 | 2.3 |
| L-12 | Hf(Bn)₄ | 1 | 1,802 | 0.06 | 686,400 | 24.0 | 0.9 |
| L-12 | Hf(Bn)₄ | 2 | 431 | 0.09 | 471,650 | 9.2 | 0.7 |
| L-12 | Zr(Bn)₄ | 1 | 607 | 0.07 | 718,400 | 35.8 | 2.2 |
| L-12 | Zr(Bn)₄ | 2 | 585 | 0.08 | 472,650 | 12.2 | 1.1 |
| L-13 | Hf(Bn)₄ | 1 | 186 | 0.12 | 417,100 | 10.8 | 4.3 |
| L-13 | Hf(Bn)₄ | 2 | 122 | 0.13 | 550,850 | 8.6 | 0.2 |
| L-13 | Zr(Bn)₄ | 1 | 132 | 0.12 | 534,950 | 29.0 | 3.8 |
| L-13 | Zr(Bn)₄ | 2 | 300 | 0.10 | 583,450 | 24.3 | 2.0 |
| L-14 | Hf(Bn)₄ | 1 | 1,086 | 0.07 | 562,100 | 14.1 | 3.6 |
| L-14 | Hf(Bn)₄ | 2 | 1,443 | 0.07 | 215,650 | 10.6 | 0.4 |
| L-14 | Zr(Bn)₄ | 1 | 225 | 0.10 | 508,250 | 27.1 | 3.0 |
| L-14 | Zr(Bn)₄ | 2 | 1,795 | 0.06 | 577,900 | 35.6 | 1.9 |
| L-15 | Hf(Bn)₄ | 1 | 94 | 0.21 | 329,000 | 14.4 | 7.7 |
| L-15 | Hf(Bn)₄ | 2 | 250 | 0.13 | 456,550 | 15.7 | 4.4 |
| L-15 | Zr(Bn)₄ | 1 | 109 | 0.13 | 629,050 | 37.2 | 3.8 |
| L-15 | Zr(Bn)₄ | 2 | 223 | 0.12 | 729,900 | 14.5 | 0.4 |
| L-16 | Hf(Bn)₄ | 1 | 245 | 0.11 | 475,600 | 11.3 | 3.5 |
| L-16 | Hf(Bn)₄ | 2 | 167 | 0.12 | 616,050 | 8.0 | 0.1 |
| L-16 | Zr(Bn)₄ | 1 | 150 | 0.13 | 487,450 | 30.3 | 3.6 |
| L-16 | Zr(Bn)₄ | 2 | 335 | 0.10 | 468,900 | 28.6 | 2.4 |
| L-17 | Hf(Bn)₄ | 1 | 254 | 0.13 | 709,300 | 18.3 | 4.2 |
| L-17 | Hf(Bn)₄ | 2 | 193 | 0.13 | 1,261,500 | 15.7 | 0.3 |
| L-17 | Zr(Bn)₄ | 1 | 133 | 0.12 | 594,650 | 27.1 | 4.4 |
| L-17 | Zr(Bn)₄ | 2 | 243 | 0.08 | 1,345,000 | 40.5 | 1.5 |
| L-18 | Hf(Bn)₄ | 1 | 679 | 0.07 | 464,050 | 8.1 | 0.6 |
| L-18 | Hf(Bn)₄ | 2 | 507 | 0.09 | 326,450 | 7.1 | 0.3 |
| L-18 | Zr(Bn)₄ | 1 | 419 | 0.07 | 577,500 | 33.8 | 2.4 |
| L-18 | Zr(Bn)₄ | 2 | 325 | 0.09 | 238,200 | 7.2 | 0.5 |
| L-19 | Hf(Bn)₄ | 1 | 1,801 | 0.04 | 194,000 | 15.8 | 4.0 |
| L-19 | Hf(Bn)₄ | 2 | 1,800 | 0.01 | | | |
| L-19 | Zr(Bn)₄ | 1 | 1,801 | 0.04 | 440,250 | 74.4 | 3.2 |
| L-19 | Zr(Bn)₄ | 2 | 1,801 | 0.01 | | | |
| L-21 | Hf(Bn)₄ | 1 | 906 | 0.07 | 611,600 | 18.2 | 1.3 |
| L-21 | Hf(Bn)₄ | 2 | 478 | 0.08 | 333,750 | 11.8 | 0.6 |
| L-21 | Zr(Bn)₄ | 1 | 453 | 0.08 | 667,500 | 36.1 | 2.8 |
| L-21 | Zr(Bn)₄ | 2 | 1,306 | 0.07 | 423,350 | 26.4 | 1.2 |

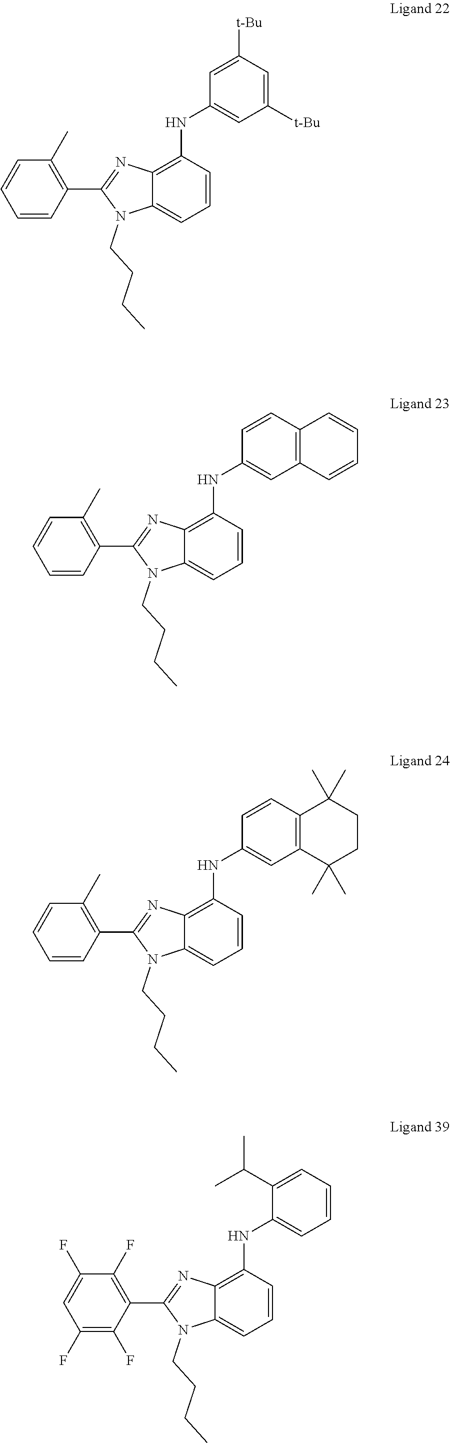 [SMILES: CCCCn1c(-c2ccccc2C)nc2c(Nc3cc(C(C)(C)C)cc(C(C)(C)C)c3)cccc21 ; CCCCn1c(-c2ccccc2C)nc2c(Nc3ccc4ccccc4c3)cccc21 ; CCCCn1c(-c2ccccc2C)nc2c(Nc3ccc4c(c3)C(C)(C)CCC4(C)C)cccc21 ; CCCCn1c(-c2c(F)c(F)cc(F)c2F)nc2c(Nc3ccccc3C(C)C)cccc21]

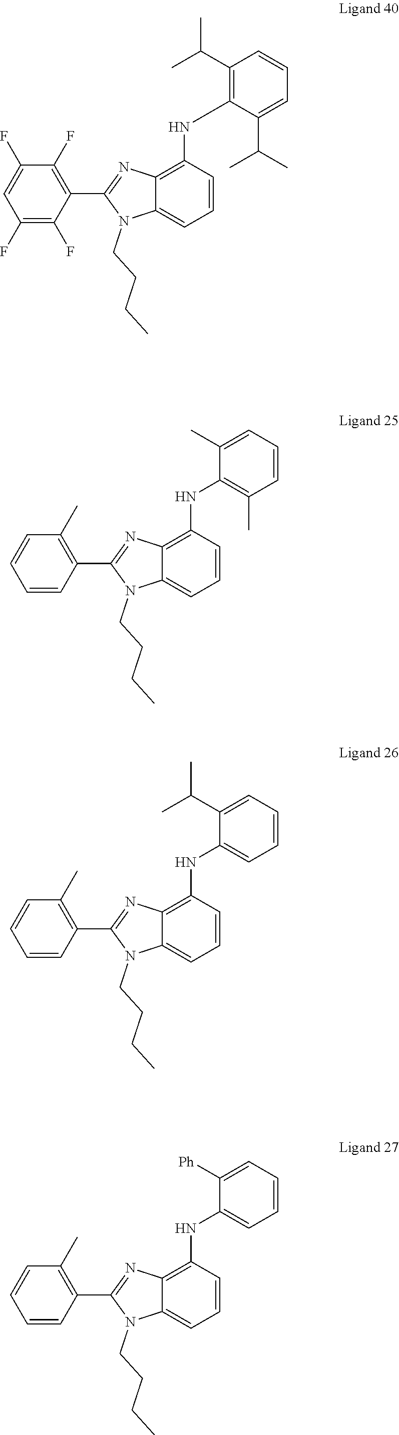 [SMILES: CCCCn1c(-c2c(F)c(F)cc(F)c2F)nc2c(Nc3c(C(C)C)cccc3C(C)C)cccc21 ; CCCCn1c(-c2ccccc2C)nc2c(Nc3c(C)cccc3C)cccc21 ; CCCCn1c(-c2ccccc2C)nc2c(Nc3ccccc3C(C)C)cccc21 ; CCCCn1c(-c2ccccc2C)nc2c(Nc3ccccc3-c3ccccc3)cccc21]

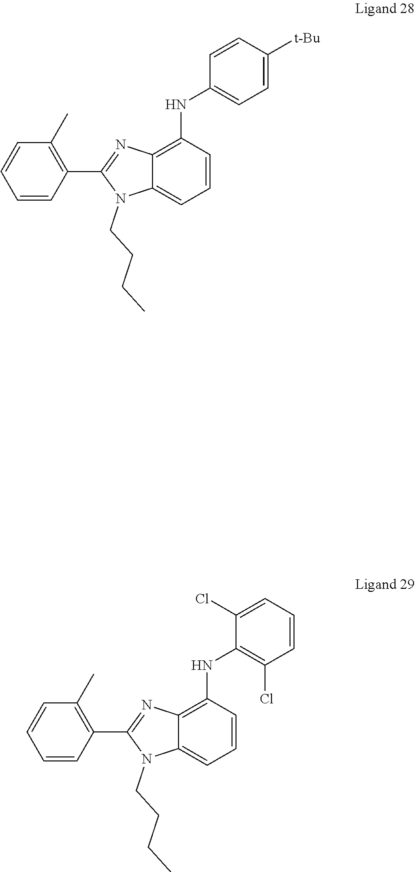 [SMILES: CCCCn1c(-c2ccccc2C)nc2c(Nc3ccc(C(C)(C)C)cc3)cccc21 ; CCCCn1c(-c2ccccc2C)nc2c(Nc3c(Cl)cccc3Cl)cccc21]

TABLE 12
PPR results for ortho-tolyl based amino-benzimidazole catalysts

| Ligand# | | L/M | Quench | Yield | Mw | | Mol % |
|---|---|---|---|---|---|---|---|
| (L-#) | Metal | ratio | (sec) | (g) | (g/mol) | PDI | Octene |
| L-22 | Hf(Bn)₄ | 1 | 432 | 0.09 | 94,035 | 4.7 | 1.8 |
| L-22 | Hf(Bn)₄ | 2 | 111 | 0.08 | 40,055 | 3.2 | 0.6 |
| L-22 | Zr(Bn)₄ | 1 | 100 | 0.12 | 128,100 | 8.1 | 2.6 |
| L-22 | Zr(Bn)₄ | 2 | 32 | 0.11 | 32,650 | 3.3 | 0.5 |
| L-23 | Hf(Bn)₄ | 1 | 323 | 0.07 | 107,450 | 6.5 | 1.8 |
| L-23 | Hf(Bn)₄ | 2 | 113 | 0.08 | 28,220 | 2.8 | 0.6 |
| L-23 | Zr(Bn)₄ | 1 | 146 | 0.10 | 154,400 | 11.9 | 1.5 |
| L-23 | Zr(Bn)₄ | 2 | 44 | 0.10 | 34,745 | 3.8 | 0.5 |
| L-24 | Hf(Bn)₄ | 1 | 158 | 0.09 | 106,600 | 6.7 | 2.0 |
| L-24 | Hf(Bn)₄ | 2 | 141 | 0.08 | 30,590 | 2.9 | 0.4 |
| L-24 | Zr(Bn)₄ | 1 | 87 | 0.12 | 115,250 | 8.8 | 2.0 |
| L-24 | Zr(Bn)₄ | 2 | 31 | 0.13 | 23,080 | 3.1 | 0.7 |
| L-39 | Hf(Bn)₄ | 1 | 418 | 0.08 | 207,900 | 4.6 | 3.0 |
| L-39 | Hf(Bn)₄ | 2 | 218 | 0.09 | 218,850 | 5.6 | 2.8 |
| L-39 | Zr(Bn)₄ | 1 | 118 | 0.13 | 195,500 | 11.9 | 3.9 |
| L-39 | Zr(Bn)₄ | 2 | 65 | 0.12 | 82,455 | 5.1 | 1.4 |
| L-40 | Hf(Bn)₄ | 1 | 38 | 0.21 | 143,550 | 7.2 | 8.9 |
| L-40 | Hf(Bn)₄ | 2 | 158 | 0.11 | 265,300 | 5.2 | 3.8 |
| L-40 | Zr(Bn)₄ | 1 | 146 | 0.12 | 257,750 | 17.8 | 3.2 |
| L-40 | Zr(Bn)₄ | 2 | 357 | 0.09 | 279,350 | 17.8 | 2.4 |
| L-25 | Hf(Bn)₄ | 1 | 34 | 0.23 | 170,850 | 6.0 | 9.8 |
| L-25 | Hf(Bn)₄ | 2 | 59 | 0.16 | 220,650 | 5.6 | 6.7 |
| L-25 | Zr(Bn)₄ | 1 | 47 | 0.17 | 240,350 | 24.3 | 4.2 |
| L-25 | Zr(Bn)₄ | 2 | 91 | 0.13 | 283,300 | 21.6 | 3.3 |
| L-26 | Hf(Bn)₄ | 1 | 48 | 0.16 | 208,800 | 3.7 | 7.4 |
| L-26 | Hf(Bn)₄ | 2 | 155 | 0.11 | 238,050 | 5.1 | 3.5 |
| L-26 | Zr(Bn)₄ | 1 | 94 | 0.16 | 215,200 | 14.8 | 3.9 |
| L-26 | Zr(Bn)₄ | 2 | 60 | 0.15 | 149,550 | 8.7 | 1.9 |
| L-27 | Hf(Bn)₄ | 1 | 61 | 0.14 | 221,300 | 5.1 | 4.3 |
| L-27 | Hf(Bn)₄ | 2 | 426 | 0.09 | 328,100 | 11.5 | 3.0 |
| L-27 | Zr(Bn)₄ | 1 | 119 | 0.14 | 328,850 | 21.8 | 3.6 |
| L-27 | Zr(Bn)₄ | 2 | 290 | 0.10 | 425,850 | 24.7 | 2.5 |
| L-28 | Hf(Bn)₄ | 1 | 284 | 0.08 | 147,350 | 6.9 | 2.9 |
| L-28 | Hf(Bn)₄ | 2 | 96 | 0.10 | 52,340 | 4.9 | 1.0 |
| L-28 | Zr(Bn)₄ | 1 | 105 | 0.12 | 136,050 | 10.7 | 2.4 |
| L-28 | Zr(Bn)₄ | 2 | 31 | 0.14 | 32,100 | 4.6 | 1.0 |
| L-29 | Hf(Bn)₄ | 1 | 429 | 0.08 | 129,400 | 3.1 | 3.7 |
| L-29 | Hf(Bn)₄ | 2 | 564 | 0.07 | 99,895 | 3.8 | 1.4 |
| L-29 | Zr(Bn)₄ | 1 | 195 | 0.10 | 199,100 | 12.9 | 3.4 |
| L-29 | Zr(Bn)₄ | 2 | 176 | 0.09 | 170,450 | 6.3 | 0.3 |

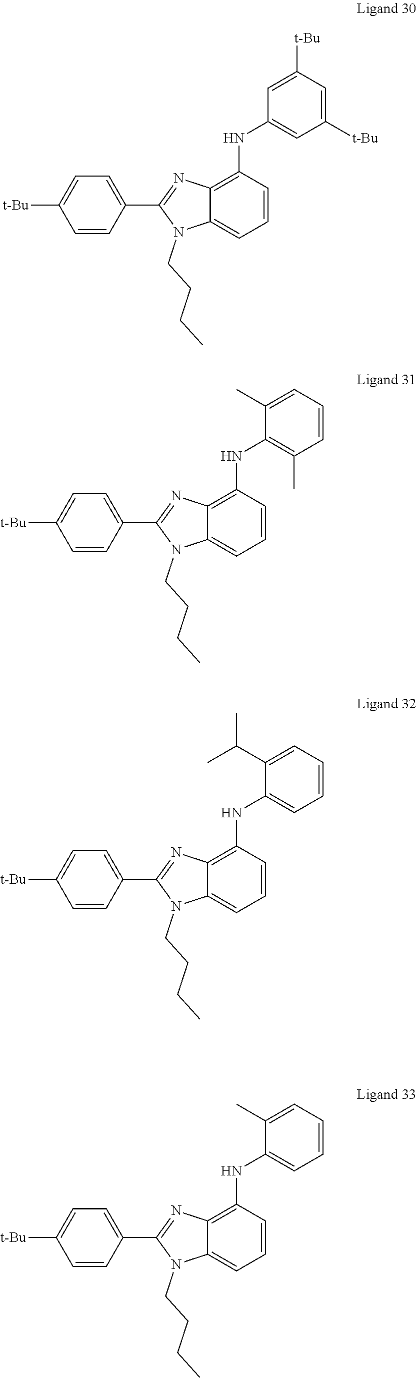 [SMILES: CCCCn1c(-c2ccc(C(C)(C)C)cc2)nc2c(Nc3cc(C(C)(C)C)cc(C(C)(C)C)c3)cccc21 ; CCCCn1c(-c2ccc(C(C)(C)C)cc2)nc2c(Nc3c(C)cccc3C)cccc21 ; CCCCn1c(-c2ccc(C(C)(C)C)cc2)nc2c(Nc3ccccc3C(C)C)cccc21 ; CCCCn1c(-c2ccc(C(C)(C)C)cc2)nc2c(Nc3ccccc3C)cccc21]

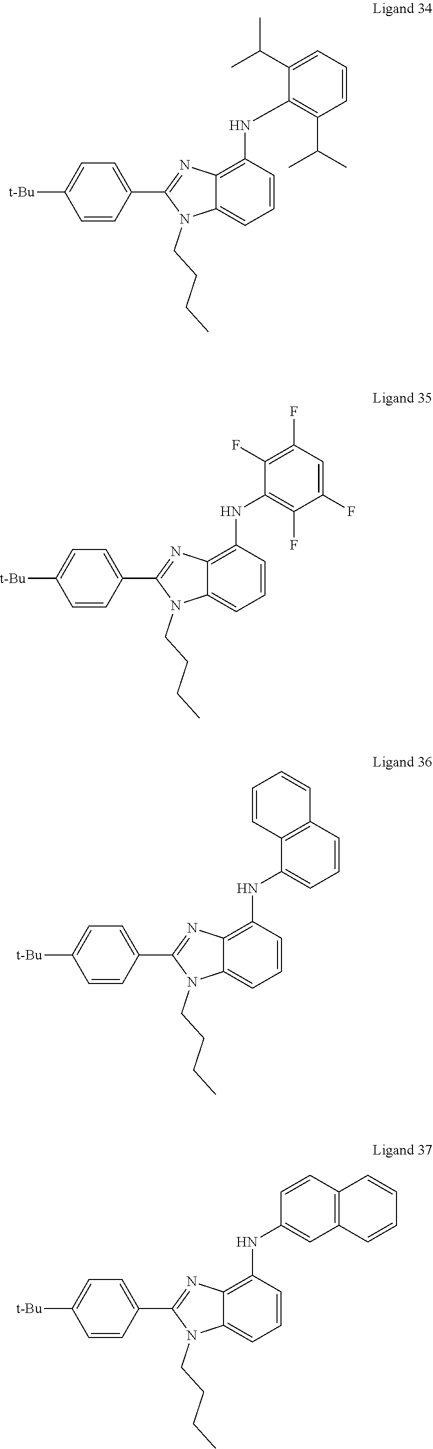 [SMILES: CCCCn1c(-c2ccc(C(C)(C)C)cc2)nc2c(Nc3c(C(C)C)cccc3C(C)C)cccc21 ; CCCCn1c(-c2ccc(C(C)(C)C)cc2)nc2c(Nc3c(F)c(F)cc(F)c3F)cccc21 ; CCCCn1c(-c2ccc(C(C)(C)C)cc2)nc2c(Nc3cccc4ccccc34)cccc21 ; CCCCn1c(-c2ccc(C(C)(C)C)cc2)nc2c(Nc3ccc4ccccc4c3)cccc21]

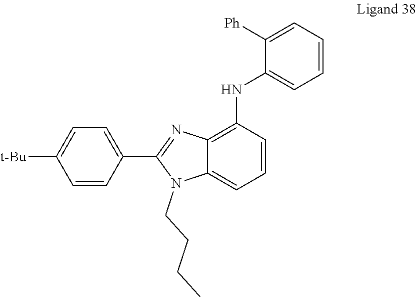 [SMILES: CCCCn1c(-c2ccc(C(C)(C)C)cc2)nc2c(Nc3ccccc3-c3ccccc3)cccc21]

TABLE 13
PPR results for carbazole-based amino-benzimidazole catalysts.

| Ligand# | | L/M | Quench | Yield | Mw | | Mol % |
|---|---|---|---|---|---|---|---|
| (L-#) | Metal | ratio | (sec) | (g) | (g/mol) | PDI | Octene |
| L-30 | Hf(Bn)₄ | 1 | 476 | 0.08 | 145,950 | 6.5 | 4.3 |
| L-30 | Hf(Bn)₄ | 2 | 593 | 0.07 | 49,470 | 7.1 | 0.8 |
| L-30 | Zr(Bn)₄ | 1 | 186 | 0.11 | 138,950 | 13.1 | 2.8 |
| L-30 | Zr(Bn)₄ | 2 | 65 | 0.10 | 26,615 | 5.6 | 0.8 |
| L-31 | Hf(Bn)₄ | 1 | 53 | 0.23 | 135,350 | 8.3 | 9.0 |
| L-31 | Hf(Bn)₄ | 2 | 1,802 | 0.04 | 226,550 | 27.1 | 3.5 |
| L-31 | Zr(Bn)₄ | 1 | 65 | 0.17 | 160,400 | 18.8 | 4.1 |
| L-31 | Zr(Bn)₄ | 2 | 1,550 | 0.07 | 280,450 | 36.3 | 1.5 |
| L-32 | Hf(Bn)₄ | 1 | 964 | 0.06 | 194,600 | 3.1 | 6.2 |
| L-32 | Hf(Bn)₄ | 2 | 1,801 | 0.05 | 196,050 | 17.6 | 2.6 |
| L-32 | Zr(Bn)₄ | 1 | 143 | 0.14 | 156,750 | 12.2 | 3.5 |
| L-32 | Zr(Bn)₄ | 2 | 234 | 0.10 | 161,450 | 10.1 | 1.4 |
| L-33 | Hf(Bn)₄ | 1 | 237 | 0.09 | 243,050 | 4.1 | 5.1 |
| L-33 | Hf(Bn)₄ | 2 | 1,801 | 0.03 | 134,000 | 17.3 | 1.5 |
| L-33 | Zr(Bn)₄ | 1 | 120 | 0.14 | 170,850 | 13.4 | 3.7 |
| L-33 | Zr(Bn)₄ | 2 | 315 | 0.10 | 239,300 | 15.1 | 1.2 |
| L-34 | Hf(Bn)₄ | 1 | 26 | 0.33 | 82,610 | 9.7 | 9.2 |
| L-34 | Hf(Bn)₄ | 2 | 71 | 0.14 | 213,450 | 4.2 | 5.0 |
| L-34 | Zr(Bn)₄ | 1 | 47 | 0.18 | 143,600 | 14.8 | 4.0 |
| L-34 | Zr(Bn)₄ | 2 | 135 | 0.11 | 242,200 | 18.8 | 3.1 |
| L-35 | Hf(Bn)₄ | 1 | 1,802 | 0.06 | 103,750 | 7.0 | 1.7 |
| L-35 | Hf(Bn)₄ | 2 | 446 | 0.08 | 42,345 | 3.2 | 0.5 |
| L-35 | Zr(Bn)₄ | 1 | 207 | 0.10 | 141,300 | 10.9 | 2.5 |
| L-35 | Zr(Bn)₄ | 2 | 96 | 0.09 | 35,680 | 3.3 | 0.6 |
| L-36 | Hf(Bn)₄ | 1 | 83 | 0.14 | 348,900 | 6.4 | 4.9 |
| L-36 | Hf(Bn)₄ | 2 | 1,628 | 0.08 | 504,250 | 28.7 | 2.1 |
| L-36 | Zr(Bn)₄ | 1 | 102 | 0.15 | 272,950 | 17.6 | 3.7 |
| L-36 | Zr(Bn)₄ | 2 | 252 | 0.12 | 399,650 | 23.5 | 2.3 |
| L-37 | Hf(Bn)₄ | 1 | 1,801 | 0.03 | 251,550 | 20.9 | 2.7 |
| L-37 | Hf(Bn)₄ | 2 | 754 | 0.07 | 71,010 | 7.5 | 0.9 |
| L-37 | Zr(Bn)₄ | 1 | 527 | 0.08 | 340,600 | 33.1 | 2.4 |
| L-37 | Zr(Bn)₄ | 2 | 267 | 0.08 | 102,345 | 13.0 | 1.0 |
| L-38 | Hf(Bn)₄ | 1 | 157 | 0.12 | 218,100 | 6.7 | 4.7 |
| L-38 | Hf(Bn)₄ | 2 | 1,802 | 0.03 | 206,550 | 41.2 | 3.4 |
| L-38 | Zr(Bn)₄ | 1 | 202 | 0.12 | 323,200 | 25.9 | 4.2 |
| L-38 | Zr(Bn)₄ | 2 | 1,407 | 0.08 | 329,150 | 44.5 | 2.7 |

### GENERAL MATERIALS

All commercial chemicals were used without further purification. Hexanes, Isopar E, and toluene that were used in the glove box were purified through a solvent purification system, then dried over molecular sieves.

1. A catalyst system comprising a metal-ligand complex according to formula (I): 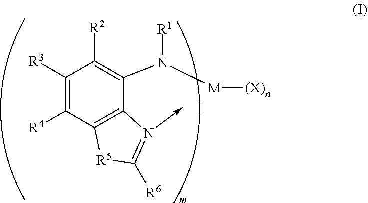
where
M is a metal chosen from titanium, zirconium, or hafnium, the metal having a formal oxidation state of +2, +3, or +4;
each X is a monodentate or bidentate ligand independently chosen from unsaturated (C₂-C₅₀)hydrocarbon, unsaturated (C₂-C₅₀)heterohydrocarbon, (C₁-C₅₀)hydrocarbyl, (C₆-C₅₀)aryl, (C₆-C₅₀)heteroaryl, cyclopentadienyl, substituted cyclopentadienyl, (C₄-C₁₂)diene, halogen, and —CH₂SiR^{C}₃;
n is 2 or 3;
m is 1 or 2;
the metal-ligand complex has 6 or fewer metal-ligand bonds;
each R¹ is independently chosen from unsubstituted (C₁-C₅₀)alkyl, substituted (C₁-C₅₀)alkyl, unsubstituted (C₆-C₅₀)aryl, or substituted (C₆-C₅₀)aryl;
each R², R³, and R⁴ is independently selected from —H, (C₁-C₅₀)hydrocarbyl, (C₁-C₅₀)heterohydrocarbyl, (C₆-C₅₀)aryl, (C₄-C₅₀)heteroaryl, halogen atom, —OR^{C}, —Si(R^{C})₃, and —Ge(R^{C})₃;
each R⁵ is selected from S, —NR^{N}, or CR^{N}₂, wherein each R^{N} is (C₁-C₂₀)hydrocarbyl or —H; and
each R⁶ is independently selected from —H, (C₁-C₅₀)hydrocarbyl, (C₁-C₅₀)heterohydrocarbyl, (C₆-C₅₀)aryl, (C₄-C₅₀)heteroaryl, —Si(R^{C})₃, and —Ge(R^{C})₃;
wherein each R^{C} is selected from the group consisting (C₁-C₃₀)hydrocarbyl or —H.

2. The catalyst system according to claim 1, wherein:
M is zirconium or hafnium;
each X is independently chosen from unsubstituted (C₁-C₁₀)alkyl, substituted (C₁-C₁₀)alkyl, (C₆-C₂₀)aryl or a halogen; and
each R¹ is independently chosen unsubstituted (C₁-C₅₀)alkyl, substituted (C₁-C₅₀)alkyl, unsubstituted (C₆-C₅₀)aryl, or substituted (C₆-C₅₀)aryl.

3. The catalyst system according to claim 1, wherein each R³, R⁴, and R⁵ is —H.

4. The catalyst system according to claim 1, wherein each R¹ is unsubstituted phenyl, substituted phenyl, unsubstituted anthracenyl, substituted anthracenyl, unsubstituted napthyl, or substituted naphtyl.

5. The catalyst system according to claim 1, wherein each R¹ is a substituted or unsubstituted phenyl.

6. The catalyst system according to claim 5, wherein the substituted phenyl is chosen from 2-methylphenyl, 2-(iso-propyl)phenyl, 2,4,6-trimethylphenyl, 2,6-di(iso-propyl)phenyl, 2,4,6-tri(iso-propyl)phenyl, 3,5-di-tert-butylphenyl, 3,5-diphenylphenyl, 2,3,5,6-tetra-fluorophenyl.

7. The catalyst system according to claim 1, wherein R⁵ is NR^{N}, where R^{N} is (C₁-C₂₀)alkyl or (C₆-C₂₀)aryl.

8. The catalyst system according to claim 7, wherein R^{N} is a linear (C₁-C₁₂)alkyl.

9. The catalyst system according to claim 1, wherein m is 2 and the metal-ligand complex has a structure according to formula (II): 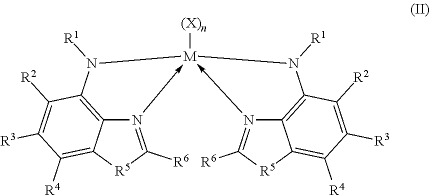
where each R¹, R², R³, R⁴, R⁵, R⁶, and X are as defined in formula (I); and n is 1 or 2.

10. The catalyst system according to claim 9, wherein:
M is zirconium or hafnium;
each X is independently chosen from (C₆-C₅₀)aryl, (C₆-C₅₀)heteroaryl, (C₁-C₅₀)hydrocarbyl, or halogen;
each R¹ and R² is independently chosen from (C₁-C₅₀)hydrocarbyl, (C₁-C₅₀)heterohydrocarbyl, (C₆-C₅₀)aryl, (C₄-C₅₀)heteroaryl, and hydrogen.

11. The catalyst system according to claim 1, wherein each X is selected from the group consisting of benzyl, methyl, chloro, or —CH₂Si(CH₃)₃.

12. The catalyst system according to claim 1, wherein each R⁶ is substituted carbazolyl, unsubstituted carbazolyl, unsubstituted phenyl, substituted phenyl, unsubstituted anthracenyl, substituted anthracenyl, unsubstituted napthyl, or substituted napthyl.

13. The catalyst system according to claim 1, wherein each R⁶ is napthyl, 2-propyl, 2-methylphenyl, 2-(iso-propyl)phenyl, 2,4,6-trimethylphenyl, 2,6-di(iso-propyl)phenyl, 2,4,6-tri(iso-propyl)phenyl, 3,5-di-tert-butylphenyl, 3,5-diphenylphenyl, or 2,7-di-tert-butylcarbazolyl.

14. The catalyst system according to claim 1, wherein each R¹ is unsubstituted phenyl, substituted phenyl, unsubstituted anthracenyl, substituted anthracenyl, unsubstituted napthyl, or substituted napthyl.

15. The catalyst system according to claim 1, wherein each R¹ is a substituted or unsubstituted phenyl.

16. The catalyst system according to claim 15, wherein the substituted phenyl is chosen from 2-methylphenyl, 2-(iso-propyl)phenyl, 2,4,6-trimethylphenyl, 2,6-di(iso-propyl)phenyl, 2,4,6-tri(iso-propyl)phenyl, 3,5-di-tert-butylphenyl, 3,5-diphenylphenyl, 2,3,5,6-tetra-fluorophenyl.

17. A process for polymerizing polymers, the process comprising: contacting ethylene and optionally one or more (C₃-C₁₂)α-olefins in the presence of a catalyst system according to claim 1 in a reactor, wherein the catalyst system further comprises an activator.

18. The process of claim 17, wherein the process further comprises a solvent.

19. The process of claim 18, wherein the reactor is a batch reactor, an autoclave reactor, or a continuous stir tank reactor.

20. A metal-ligand complex selected from: 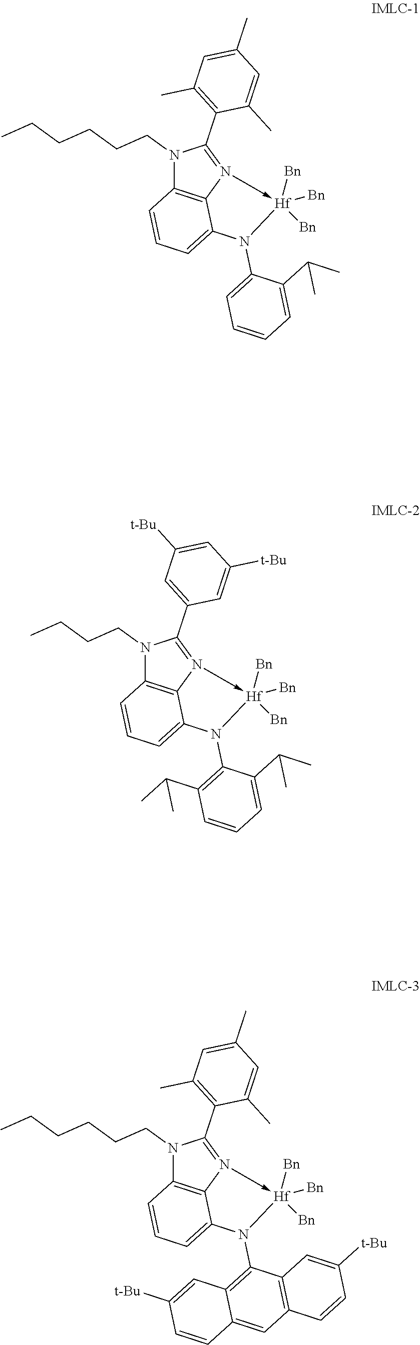 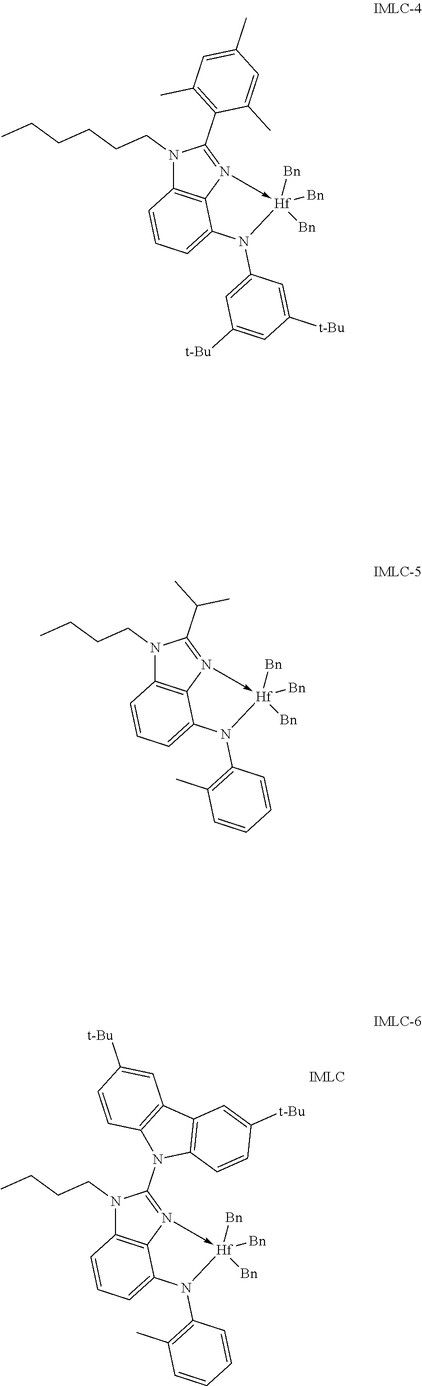 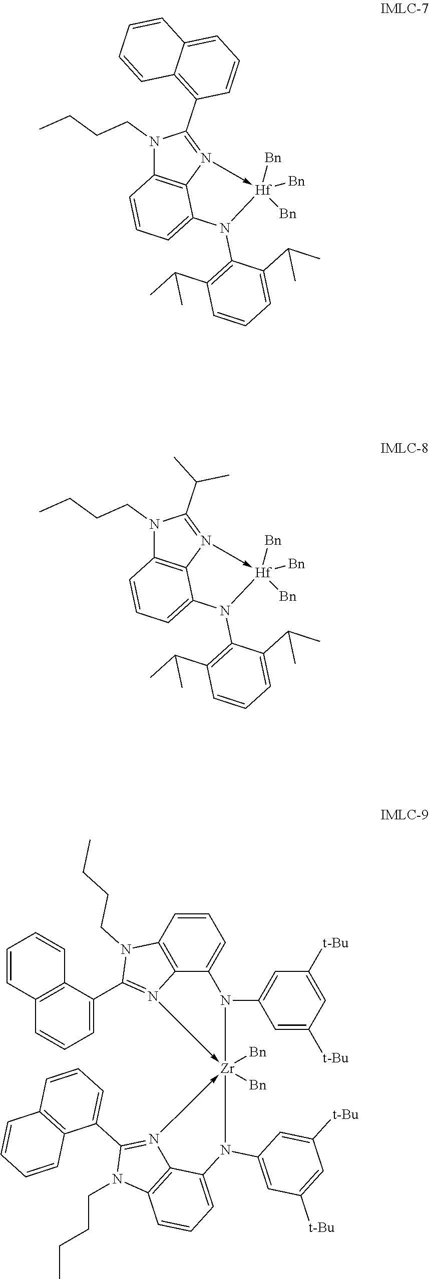 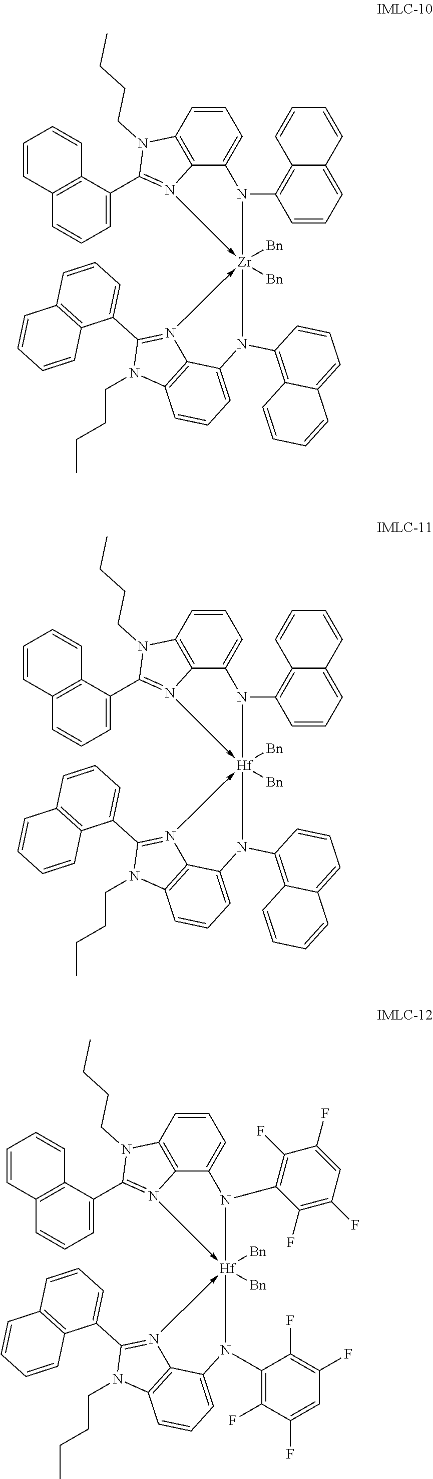 [chemical structure]
